(19) United States
(12) Patent Application Publication
(10) Pub. No.: US 2023/0310469 A1
(43) Pub. Date: Oct. 5, 2023

(54) METHODS OF TREATING SYMPTOMS OF CORONAVIRUS INFECTION WITH TOLL-LIKE-RECEPTOR AGONISTS

(71) Applicant: Accencio LLC, Philadelphia, PA (US)

(72) Inventors: Kevin Brown, Philadelphia, PA (US); Kevin Brogle, Cream Ridge, NJ (US)

(21) Appl. No.: 18/023,600

(22) PCT Filed: Aug. 30, 2021
(86) PCT No.: PCT/US2021/048151

§ 371 Date: Feb. 27, 2023

Related U.S. Application Data
(60) Provisional application No. 63/071,905, filed on Aug. 28, 2020.

(51) Int. Cl. A61K 31/683; A61K 31/708; A61K 31/55; A61K 31/4375; A61K 31/522; A61K 9/00

(52) U.S. Cl. CPC A61K 31/683; A61K 31/708; A61K 31/55; A61K 31/4375; A61K 31/522; A61K 9/0014

(57) ABSTRACT

The present disclosure relates to methods of treating at least one symptom of a coronavirus infection, or preventing an acute inflammatory response, e.g., a cytokine storm in a coronavirus patient, in particular a SARS-CoV-19 patient, by administering a Toll-Like-Receptor (TLR) agonist, in particular a TLR-7 or TLR-8 agonist.

### FIELD

The present disclosure relates to methods of treating symptoms of a coronavirus infection, e.g., SARS-CoV-19, by administering a Toll-Like-Receptor (TLR) agonist, in particular a TLR-7 or TLR-8 agonist.

### BACKGROUND

The novel virus 2019-nCoV (SARS-CoV-19, COVID-19), is the third well-known coronavirus to cross species to infect human populations in the past two decades. The previous two are the severe acute respiratory syndrome coronavirus (SARS-CoV) outbreak in 2002 and the Middle East respiratory syndrome coronavirus (MERS-CoV) outbreak in 2012. Like SARS-CoV and MERs-CoV, SARS-CoV-19 causes severe respiratory illness, and is highly transmissible from human-to-human. On Mar. 11, 2020, the World Health Organization (WHO) declared SARS-CoV-19 a global pandemic. Since then, over 20 million people have been infected, and over 750,000 people have died worldwide from the virus. In the United States alone there have been over 5 million infections to date, with over 160,000 deaths.

Most of the critically ill patients do not develop severe clinical manifestations in early stages of the diseases; however, these patients rapidly deteriorate in the later stages of the disease, presenting with Acute Respiratory Distress Syndrome (ARDS) and multiple-organ failure, resulting in death within a short time. Evidence suggests that proinflammatory responses play a role in the pathogenesis of SARS-CoV-19 and other coronaviruses. Dysregulations of cytokine-chemokine responses cause the immune system to become hyperactive and induce a condition called a cytokine storm, which is considered to be one of the major causes of ARDS and multiple-organ failure in these patients. Targeting cytokines during the management of SARS-CoV-19 patients could improve survival rates and reduce mortality.

To date, no treatment or vaccine has been approved to combat SARS-CoV-19.

There is therefore an urgent and unmet need for effective means to combat the symptoms of SARS-CoV-19 and other coronaviruses such as SARS-CoV and MERS-CoV.

### SUMMARY

The present disclosure relates to methods of treating one or more symptoms of a coronavirus infection, particularly SARS-CoV-19. The present disclosure further relates to methods of treating or preventing an acute inflammatory response, e.g., a cytokine storm in a coronavirus patient, by administering a Toll-Like-Receptor (TLR) agonist, in particular a TLR-7 or TLR-8 agonist.

Toll-like receptors (TLRs) can recognize pathogens and are significantly expressed in immune cells. In particular, TLR-7 and TLR-8 are innate immune sensors that detect single stranded (ss) RNA from viruses such as SARS-Co-2. The present disclosure is based on the discovery that TLR-7 and TLR-8 agonists may have therapeutic utility in the treatment of coronavirus symptoms, in particular in reducing inflammation and preventing cytokine storms in patients with coronavirus infections, in particular SARS-CoV-19.

Thus, in some embodiments, the present disclosure relates to a method of treating or alleviating at least one symptom of a coronavirus infection in a subject, by administering to the subject a therapeutically effective amount of a Toll-Like Receptor (TLR)-7 or TLR-8 agonist. In some embodiments, the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure. In some embodiments, the subject is a human.

In some embodiments, the present disclosure relates to a method of treating an acute inflammatory condition in a subject infected with a coronavirus, the method comprising the step of administering to the subject a therapeutically effective amount of a Toll-Like Receptor (TLR)-7 or TLR-8 agonist. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of a Toll-Like Receptor (TLR)-7 or TLR-8 agonist. In some embodiments, the subject is a human.

In some embodiments, the present disclosure relates to a method of reducing or arresting viral load in a subject infected with a coronavirus, the method comprising the step of administering to the subject a therapeutically effective amount of a Toll-Like Receptor (TLR)-7 or TLR-8 agonist. In some embodiments, the subject is a human.

In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

In some embodiments, the TLR-7 or TLR-8 agonist is selected from the group consisting of: 4-amino-2-butoxy-8-(3-(pyrrolidin-1-ylmethyl)benzyl)-7,8-dihydropteridin-6(5H)-one (vesatolimod); 7-allyl-2-amino-9-((2R,3R,4S,5R)-3,4-dihydroxy-5-(hydroxymethyl) tetrahydrofuran-2-yl)-7,9-dihydro-1H-purine-6,8-dione (loxoribine); 2-amino-N,N-dipropyl-8-(4-(pyrrolidine-1-carbonyl)phenyl)-3H-benzo[b]azepine-4-carboxamide (motolimod); 3-[5-amino-2-[2-[4-[2-(3,3-difluoro-3-phosphonopropoxy)ethoxy]-2-methylphenyl]ethyl]benzo[f][1,7]naphthyridin-8-yl]propanoic acid (LHC-165); 1-(4-amino-2-(ethoxymethyl)-1H-imidazo[4,5-c]quinolin-1-yl)-2-methylpropan-2-ol (resiquimod); 6-amino-2-(pentan-2-yloxy)-9-(5-(piperidin-1-yl)pentyl)-7,9-dihydro-8H-purin-8-one (GSK-2245035); N-(4-((4-amino-2-butyl-1H-imidazo[4,5-c]quinolin-1-yl)oxy)butyl)stearamide (telratolimod); N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione (isatoribine); N-[4-(4-amino-2-ethyl-1H-imidazo[4,5-c]quinolin-1-yl)butyl]-methanesulfonamide (3M-852A); 1-isobutyl-1H-imidazo[4,5-c]quinolin-4-amine (Imiquimod); 2-(4-((6-amino-2-(2-methoxyethoxy)-8-oxo-7H-purin-9(8H)methyl)benzamido)-ethyl-2,3-bis(oleoyloxy)propyl phosphate (TMX-202); methyl 2-(3-(((3-(6-amino-2-butoxy-8-oxo-7,8-dihydro-9H-purin-9-yl)propyl)(3-morpholinopropyl)amino)methyl) phenyl) acetate (AZD-8848); 4-amino-1-benzyl-6-trifluoromethyl-1,3-dihydro-imidazo[4,5-c]pyridine-2-one (PF-4171455); 1-isobutyl-1H-imidazo[4,5-c][1,5]naphthyridin-4-amine (epetirimod); 1-isobutyl-2-methyl-1H-imidazo[4,5-c][1,5]naphthyridin-4-amine (sotirimod); 1-isobutyl-2-methyl-1H-imidazo[4,5-c][1,5]naphthyridin-4-amine (BIIB-021); (((1-(6-amino-9H-purin-9-yl)propan-2-yl)oxy)methyl)phosphonic acid (tenofovir); a tenefovir prodrug selected from the group consisting of tenofovir disoproxil and tenofovir exalidex; and salts and any combinations thereof.

In some embodiments, the TLR-7 or TLR-8 agonist is selected from the group consisting of a compound of any one of Table 1, Table 2, Table 3, Table 4, Table 5, Table 6, Table 7, Table 8, Table 9, Table 10, Table 11, Table 12, Table 13, Table 14, Table 15 or Table 16.

In some embodiments, TLR-7 or TLR-8 agonist is administered according to a dose regimen selected from the group consisting of once daily (q.d.), twice daily (b.i.d.) thrice daily (t.i.d.), once a week, twice a week, three times a week, once every 2 weeks, once every three weeks, or once a month.

In some embodiments, the TLR-7 or TLR-8 agonist is administered in a pharmaceutical composition, wherein the composition further comprises at least one pharmaceutically acceptable excipient.

In some embodiments, the TLR-7 or TLR-8 agonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

In some embodiments, the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

In some embodiments, the present disclosure relates to a topical pharmaceutical composition in a form selected from the group consisting of ointment, a gel, a drop, a patch and a cream, the composition comprising a TLR-7 or TLR-8 agonist and at least one topically acceptable excipient, wherein the TLR-7 or TLR-7 agonist is selected from the group 4-amino-2-butoxy-8-(3-(pyrrolidin-1-ylmethyl)benzyl)-7,8-dihydropteridin-6(5H)-one (vesatolimod); 7-allyl-2-amino-9-((2R,3R,4S,5R)-3,4-dihydroxy-5-(hydroxymethyl) tetrahydrofuran-2-yl)-7,9-dihydro-1H-purine-6,8-dione (loxoribine); 2-amino-N,N-dipropyl-8-(4-(pyrrolidine-1-carbonyl)phenyl)-3H-benzo[b]azepine-4-carboxamide (motolimod); 3-[5-amino-2-[2-[4-[2-(3,3-difluoro-3-phosphonopropoxy)ethoxy]-2-methylphenyl]ethyl]benzo[f][1,7]naphthyridin-8-yl]propanoic acid (LHC-165); 1-(4-amino-2-(ethoxymethyl)-1H-imidazo[4,5-c]quinolin-1-yl)-2-methylpropan-2-ol (resiquimod); 6-amino-2-(pentan-2-yloxy)-9-(5-(piperidin-111)pentyl)-7,9-dihydro-8H-purin-8-one (GSK-2245035); N-(4-((4-amino-2-butyl-1H-imidazo[4,5-c]quinolin-1-yl)oxy)butyl)stearamide (telratolimod); N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione (isatoribine); N-[4-(4-amino-2-ethyl-1H-imidazo[4,5-c]quinolin-1-yl)buty]-methanesulfonamide (3M-852A); 1-isobutyl-1H-imidazo[4,5-c]quinolin-4-amine (Imiquimod); 2-(4-((6-amino-2-(2-methoxyethoxy)-8-oxo-7H-purin-9(8H)methyl)benzamido)-ethyl-2,3-bis(oleoyloxy)propyl phosphate (TMX-202); methyl 2-(3-(((3-(6-amino-2-butoxy-8-oxo-7,8-dihydro-9H-purin-9-yl)propyl)(3-morpholinopropyl)amino)methyl) phenyl) acetate (AZD-8848); 4-amino-1-benzyl-6-trifluoromethyl-1,3-dihydro-imidazo[4,5-c]pyridine-2-one (PF-4171455); 1-isobutyl-1H-imidazo[4,5-c][1,5]naphthyridin-4-amine (epetirimod); 1-isobutyl-2-methyl-1H-imidazo[4,5-c][1,5]naphthyridin-4-amine (sotirimod); 1-isobutyl-2-methyl-1H-imidazo[4,5-c][1,5]naphthyridin-4-amine (B116-021); (((1-(6-amino-9H-purin-9-yl)propan-2-yl)oxy)methyl)phosphonic acid (tenofovir); a tenefovir prodrug selected from the group consisting of tenofovir disoproxil and tenofovir exalidex; and salts and any combinations thereof.

In some embodiments, the present invention relates to topical pharmaceutical composition in a form selected from the group consisting of ointment, a gel, a drop, a patch and a cream, the composition comprising a TLR-7 or TLR-8 agonist and at least one topically acceptable excipient, wherein the TLR-7 or TLR-7 agonist is selected from the group consisting of a compound of any one of Table 1, Table 2, Table 3, Table 4, Table 5, Table 6, Table 7, Table 8, Table 9, Table 10, Table 11, Table 12, Table 13, Table 14, Table 15 or Table 16.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the present disclosure will become apparent to those skilled in the art from this detailed description

### DETAILED DESCRIPTION OF THE INVENTION

As used herein and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

As used herein and as well understood in the art, the term an “effective amount,” “sufficient amount” or “therapeutically effective amount” of an agent as used herein interchangeably, is that amount sufficient to effectuate beneficial or desired results, including preclinical and/or clinical results and, as such, an “effective amount” or its variants depends upon the context in which it is being applied. The response is in some embodiments preventative, in others therapeutic, and in others a combination thereof. The term “effective amount” also includes the amount of a compound of the disclosure, which is “therapeutically effective” and which avoids or substantially attenuates undesirable side effects.

As used herein and as well known in the art, and unless otherwise defined, the term “subject” means an animal, including but not limited a human, monkey, cow, horse, sheep, pig, chicken, turkey, quail, cat, dog, mouse, rat, rabbit, or guinea pig. In one embodiment, the subject is a mammal and in another embodiment the subject is a human coronavirus patient.

### Toll-Like Receptor (TLR) Agonists

Toll-like receptors (TLRs) can recognize pathogens and are significantly expressed in immune cells. In humans, the TLR family comprises ten members (TLR1-TLR10), which are expressed in innate immune cells such as macrophages as well as in epithelial and fibroblast cells. Activation of TLRs can be induced by a multitude of pathogen-associated molecular patterns (PAMPs) present in bacteria, viruses and other foreign organisms. TLRs play a major role in the initiation of innate immune responses, with the production of inflammatory cytokines, type I interferon (IFN) and other mediators. TLR activation causes nuclear translocation of the transcription factors NF-κB, IRF-3 and IRF-7, with production of innate pro-inflammatory cytokines (IL-1, IL-6, TNF-α) and type I IFN-α/β, which are essential for anti-viral responses. SARS-CoV-19 may prevent a successful immune response in infected individuals who progress to severe pathology via inhibition of the TNF-receptor-associated factors (TRAF)-3 and -6, which play an essential role in inducing interferon regulatory transcription factor (IRF)-3/7 in response to TLR-7 activation.

TLRs may be involved both in the initial failure of viral clearance and in the subsequent development of the deadly clinical manifestations of severe SARS-Cov-19, i.e., ARDS with fatal respiratory failure. In particular, TLR-7 and TLR-8 recognize viral single-stranded RNA and are therefore, likely to be implicated in clearance of SARS-CoV-19. Thus, in some embodiments, agonists of TLR-7 and TLR-8 may prevent onset of severe SARS-CoV-19 symptoms.

The present disclosure is based on the discovery that TLR-7 and TLR-8 agonists may have therapeutic utility in the treatment of coronavirus symptoms, in particular in preventing cytokine storms in critical patients with coronavirus infections, in particular SARS-CoV-19.

In some embodiments, the compound is Vesatolimod, a TLR-7 agonist which is chemically designated 4-amino-2-butoxy-8-(3-(pyrrolidin-1-ylmethyl)benzyl)-7,8-dihydropteridin-6(5H)-one. Vesatolimod is currently in clinical trials for the treatment HIV-1 (AIDS), hepatitis B (HBV) and hepatitis-C. Vesatolimod is represented by the structure:

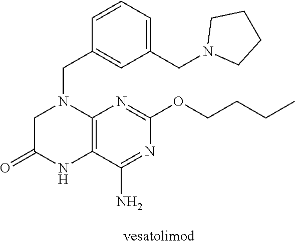 [SMILES: CCCCOc1nc(N)c2c(n1)N(Cc1cccc(CN3CCCC3)c1)CC(=O)N2]

In some embodiments, vesatolimod is administered orally. In other embodiments, vesatolimod is administered as a tablet. In some embodiments, vesatolimod is administered once a week. In other embodiments, vesatolimod is administered once every 2 weeks. Suitable dosing regiments range from about 1 mg to about 10 mg per dose, e.g., 1 mg, 2 mg, 3 mg, 4 mg, 5 mg, 6 mg, 7 mg, 8 mg, 9 mg or 10 mg, administered once a week or once every 2 weeks.

Vesatolimod and/or structurally related compounds are described in PCT International Patent Application WO 2010/077613, the contents of which are hereby incorporated by reference for all purposes and the specific purposes identified herein. In some embodiments, such compounds are represented by any one or more of the structures shown in Table 1. Any one of the compounds depicted in Table 1 is suitable for use in the methods of the present disclosure.

TABLE 1
Compound

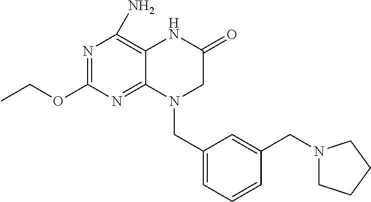 [SMILES: CCOc1nc(N)c2c(n1)N(Cc1cccc(CN3CCCC3)c1)CC(=O)N2]

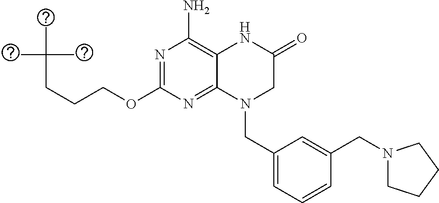 [SMILES: CC(C)(C)CCCOc1nc(N)c2c(n1)N(Cc1cccc(CN3CCCC3)c1)CC(=O)N2]

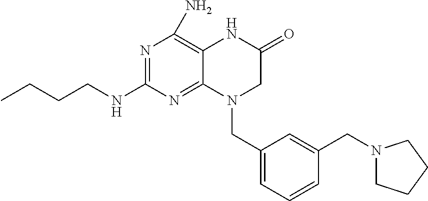 [SMILES: CCCCNc1nc(N)c2c(n1)N(Cc1cccc(CN3CCCC3)c1)CC(=O)N2]

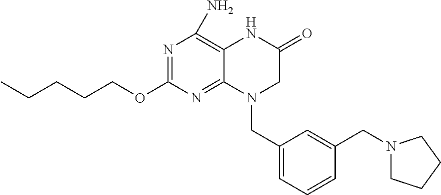 [SMILES: CCCCCOc1nc(N)c2c(n1)N(Cc1cccc(CN3CCCC3)c1)CC(=O)N2]

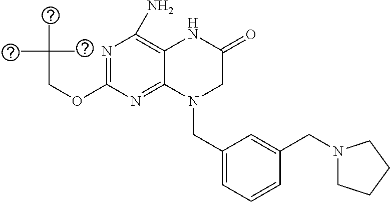 [SMILES: CC(C)(C)COc1nc(N)c2c(n1)N(Cc1cccc(CN3CCCC3)c1)CC(=O)N2]

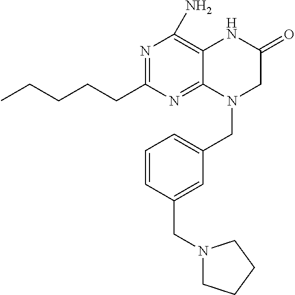 [SMILES: CCCCCc1nc(N)c2c(n1)N(Cc1cccc(CN3CCCC3)c1)CC(=O)N2]

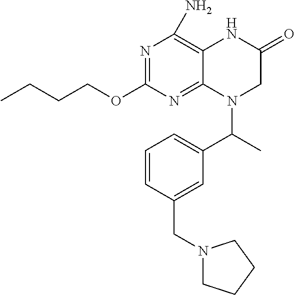 [SMILES: CCCCOc1nc(N)c2c(n1)N(C(C)c1cccc(CN3CCCC3)c1)CC(=O)N2]

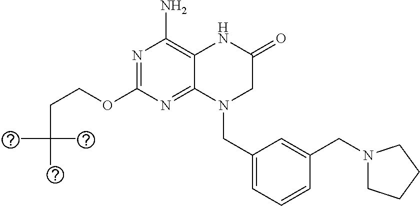 [SMILES: CC(C)(C)CCOc1nc(N)c2c(n1)N(Cc1cccc(CN3CCCC3)c1)CC(=O)N2]

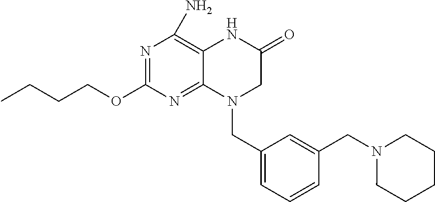 [SMILES: CCCCOc1nc(N)c2c(n1)N(Cc1cccc(CN3CCCCC3)c1)CC(=O)N2]

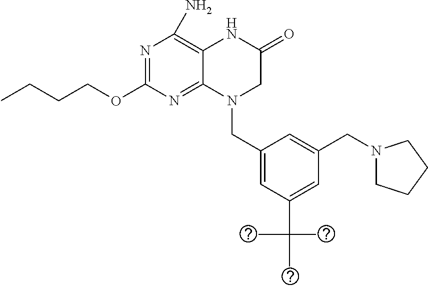 [SMILES: CCCCOc1nc(N)c2c(n1)N(Cc1cc(CN3CCCC3)cc(C(C)(C)C)c1)CC(=O)N2]

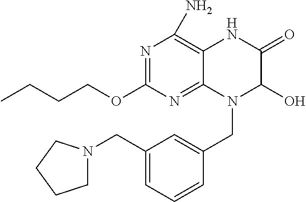 [SMILES: CCCCOc1nc(N)c2c(n1)N(Cc1cccc(CN3CCCC3)c1)C(O)C(=O)N2]

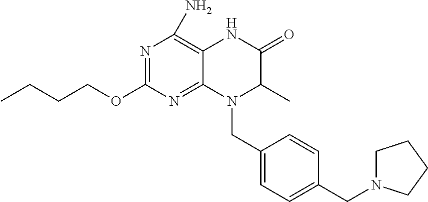 [SMILES: CCCCOc1nc(N)c2c(n1)N(Cc1ccc(CN3CCCC3)cc1)C(C)C(=O)N2]

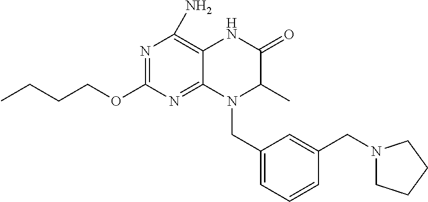 [SMILES: CCCCOc1nc(N)c2c(n1)N(Cc1cccc(CN3CCCC3)c1)C(C)C(=O)N2]

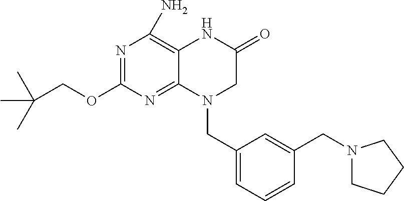 [SMILES: CC(C)(C)COc1nc(N)c2c(n1)N(Cc1cccc(CN3CCCC3)c1)CC(=O)N2]

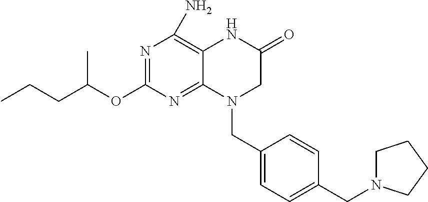 [SMILES: CCCC(C)Oc1nc(N)c2c(n1)N(Cc1ccc(CN3CCCC3)cc1)CC(=O)N2]

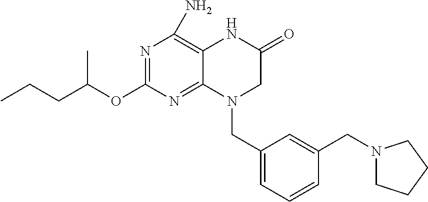 [SMILES: CCCC(C)Oc1nc(N)c2c(n1)N(Cc1cccc(CN3CCCC3)c1)CC(=O)N2]

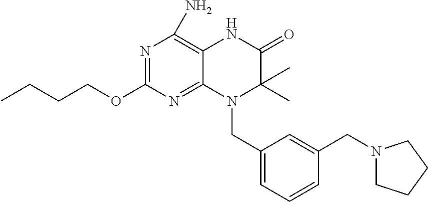 [SMILES: CCCCOc1nc(N)c2c(n1)N(Cc1cccc(CN3CCCC3)c1)C(C)(C)C(=O)N2]

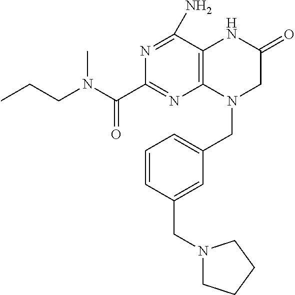 [SMILES: CCCN(C)C(=O)c1nc(N)c2c(n1)N(Cc1cccc(CN3CCCC3)c1)CC(=O)N2]

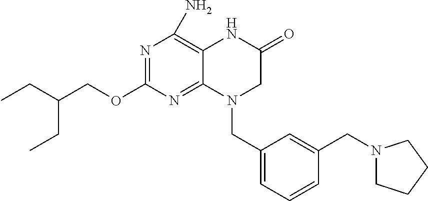 [SMILES: CCC(CC)COc1nc(N)c2c(n1)N(Cc1cccc(CN3CCCC3)c1)CC(=O)N2]

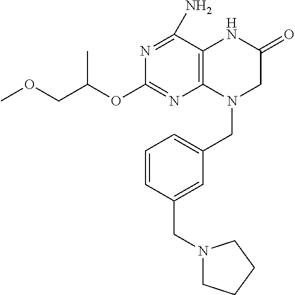 [SMILES: COCC(C)Oc1nc(N)c2c(n1)N(Cc1cccc(CN3CCCC3)c1)CC(=O)N2]

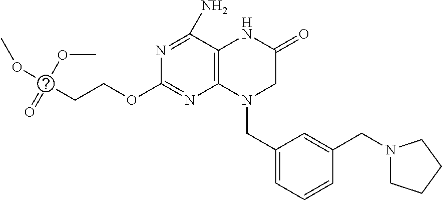

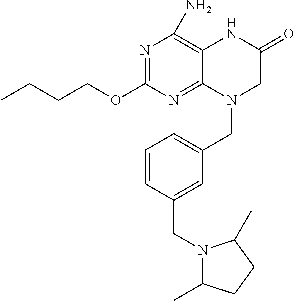 [SMILES: CCCCOc1nc(N)c2c(n1)N(Cc1cccc(CN3C(C)CCC3C)c1)CC(=O)N2]

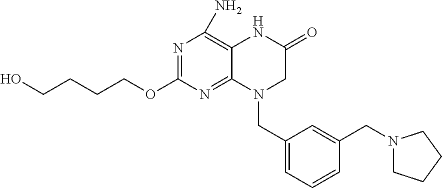 [SMILES: Nc1nc(OCCCCO)nc2c1NC(=O)CN2Cc1cccc(CN2CCCC2)c1]

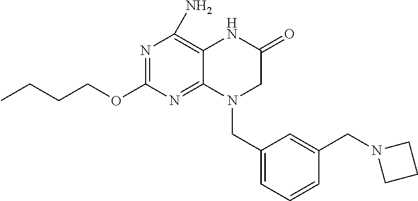 [SMILES: CCCCOc1nc(N)c2c(n1)N(Cc1cccc(CN3CCC3)c1)CC(=O)N2]

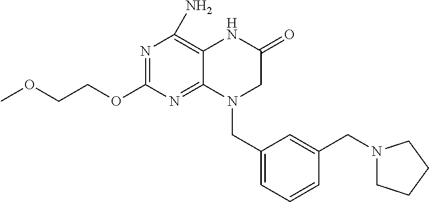 [SMILES: COCCOc1nc(N)c2c(n1)N(Cc1cccc(CN3CCCC3)c1)CC(=O)N2]

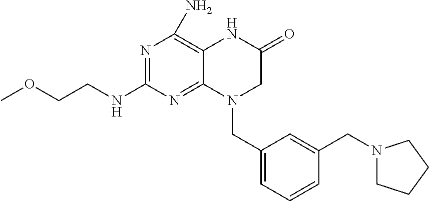 [SMILES: COCCNc1nc(N)c2c(n1)N(Cc1cccc(CN3CCCC3)c1)CC(=O)N2]

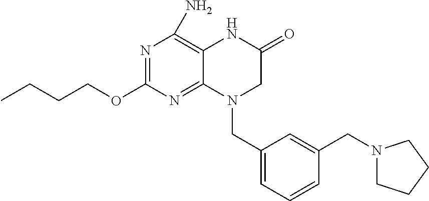 [SMILES: CCCCOc1nc(N)c2c(n1)N(Cc1cccc(CN3CCCC3)c1)CC(=O)N2]

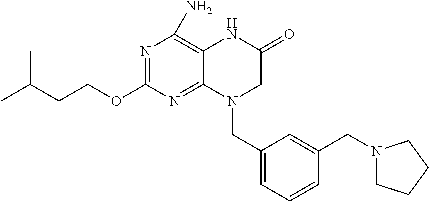 [SMILES: CC(C)CCOc1nc(N)c2c(n1)N(Cc1cccc(CN3CCCC3)c1)CC(=O)N2]

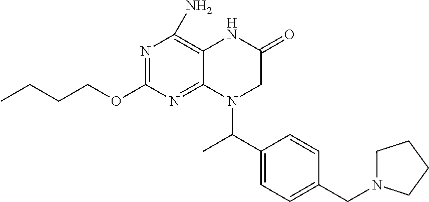 [SMILES: CCCCOc1nc(N)c2c(n1)N(C(C)c1ccc(CN3CCCC3)cc1)CC(=O)N2]

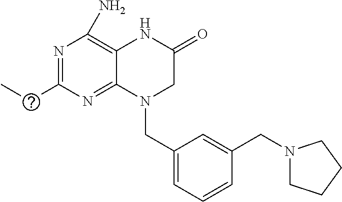 [SMILES: CCc1nc(N)c2c(n1)N(Cc1cccc(CN3CCCC3)c1)CC(=O)N2]

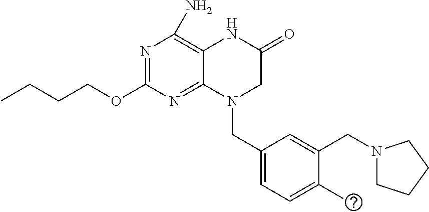 [SMILES: CCCCOc1nc(N)c2c(n1)N(Cc1ccc(C)c(CN3CCCC3)c1)CC(=O)N2]

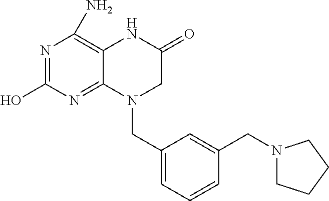 [SMILES: Nc1nc(O)nc2c1NC(=O)CN2Cc1cccc(CN2CCCC2)c1]

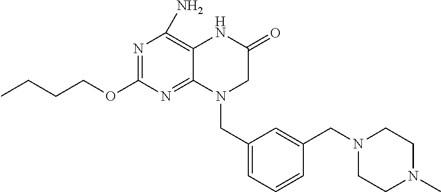 [SMILES: CCCCOc1nc(N)c2c(n1)N(Cc1cccc(CN3CCN(C)CC3)c1)CC(=O)N2]

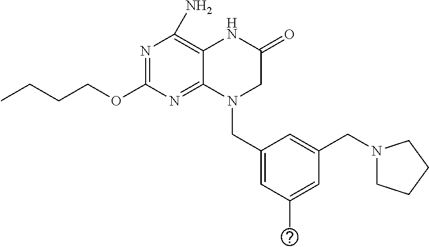 [SMILES: CCCCOc1nc(N)c2c(n1)N(Cc1cc(C)cc(CN3CCCC3)c1)CC(=O)N2]

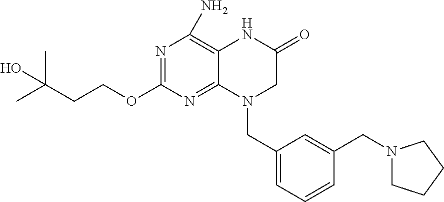 [SMILES: CC(C)(O)CCOc1nc(N)c2c(n1)N(Cc1cccc(CN3CCCC3)c1)CC(=O)N2]

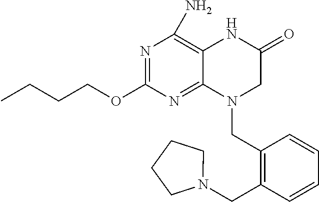 [SMILES: CCCCOc1nc(N)c2c(n1)N(Cc1ccccc1CN1CCCC1)CC(=O)N2]

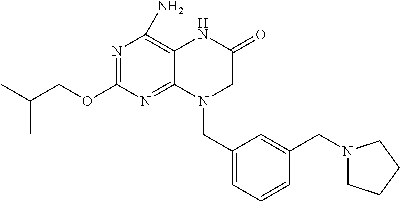 [SMILES: CC(C)COc1nc(N)c2c(n1)N(Cc1cccc(CN3CCCC3)c1)CC(=O)N2]

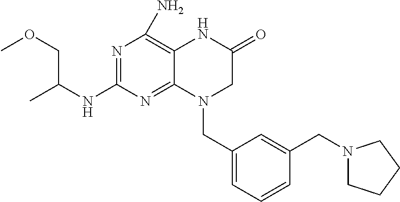 [SMILES: COCC(C)Nc1nc(N)c2c(n1)N(Cc1cccc(CN3CCCC3)c1)CC(=O)N2]

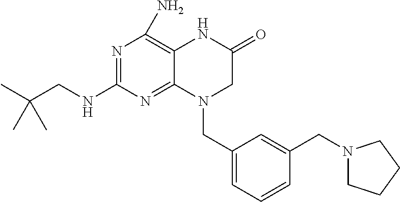 [SMILES: CC(C)(C)CNc1nc(N)c2c(n1)N(Cc1cccc(CN3CCCC3)c1)CC(=O)N2]

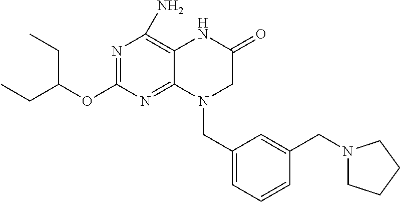 [SMILES: CCC(CC)Oc1nc(N)c2c(n1)N(Cc1cccc(CN3CCCC3)c1)CC(=O)N2]

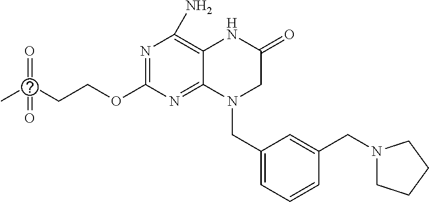

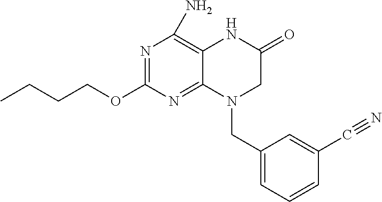 [SMILES: CCCCOc1nc(N)c2c(n1)N(Cc1cccc(C#N)c1)CC(=O)N2]

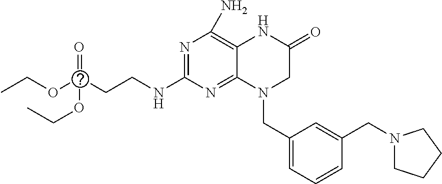

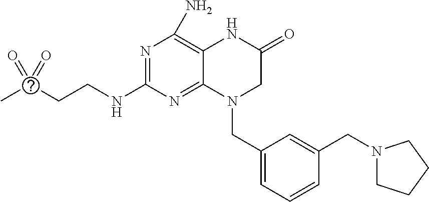

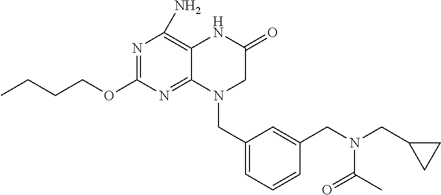 [SMILES: CCCCOc1nc(N)c2c(n1)N(Cc1cccc(CN(CC3CC3)C(C)=O)c1)CC(=O)N2]

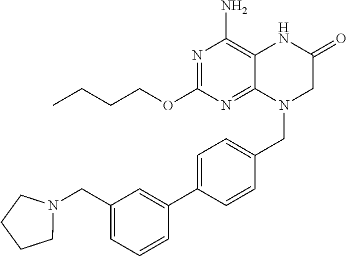 [SMILES: CCCCOc1nc(N)c2c(n1)N(Cc1ccc(-c3cccc(CN4CCCC4)c3)cc1)CC(=O)N2]

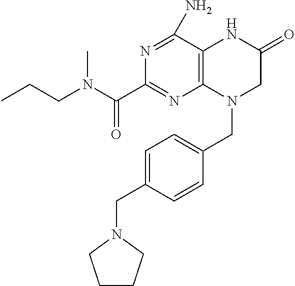 [SMILES: CCCN(C)C(=O)c1nc(N)c2c(n1)N(Cc1ccc(CN3CCCC3)cc1)CC(=O)N2]

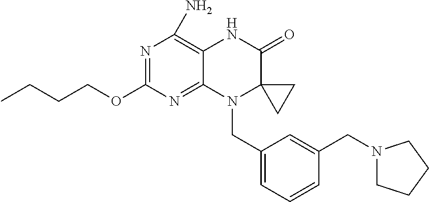 [SMILES: CCCCOc1nc(N)c2c(n1)N(Cc1cccc(CN3CCCC3)c1)C1(CC1)C(=O)N2]

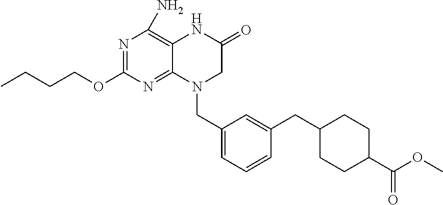 [SMILES: CCCCOc1nc(N)c2c(n1)N(Cc1cccc(CC3CCC(C(=O)OC)CC3)c1)CC(=O)N2]

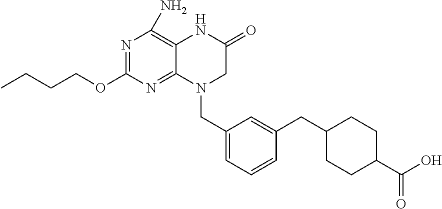 [SMILES: CCCCOc1nc(N)c2c(n1)N(Cc1cccc(CC3CCC(C(=O)O)CC3)c1)CC(=O)N2]

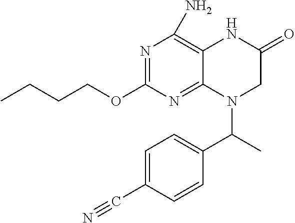 [SMILES: CCCCOc1nc(N)c2c(n1)N(C(C)c1ccc(C#N)cc1)CC(=O)N2]

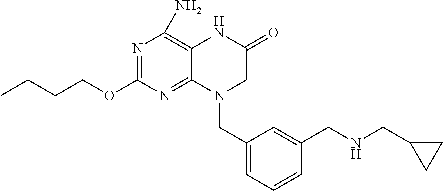 [SMILES: CCCCOc1nc(N)c2c(n1)N(Cc1cccc(CNCC3CC3)c1)CC(=O)N2]

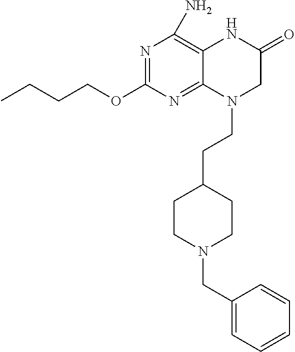 [SMILES: CCCCOc1nc(N)c2c(n1)N(CCC1CCN(Cc3ccccc3)CC1)CC(=O)N2]

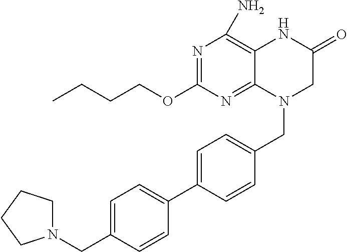 [SMILES: CCCCOc1nc(N)c2c(n1)N(Cc1ccc(-c3ccc(CN4CCCC4)cc3)cc1)CC(=O)N2]

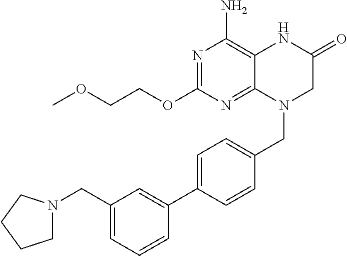 [SMILES: COCCOc1nc(N)c2c(n1)N(Cc1ccc(-c3cccc(CN4CCCC4)c3)cc1)CC(=O)N2]

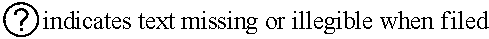

In some embodiments, the compound is Loxoribine, which is chemically designated 7-allyl-2-amino-9-((2R,3R,4S,5R)-3,4-dihydroxy-5-(hydroxymethyl)tetrahydrofuran-2-yl)-7,9-dihydro-1H-purine-6,8-dione. Loxoribine is a guanosine analogue having activity as a TLR-7 agonist. Loxoribine has been tested in clinical trials for advanced cancers. Loxoribine is represented by the structure:

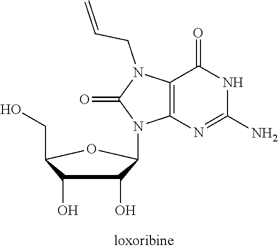

In some embodiments, loxoribine is administered at a dose ranging from 1 mg/kg to about 10 mg/kg, e.g., 1 mg/kg, 2 mg/kg, 3 mg/kg, 4 mg/kg, 5 mg/kg, 6 mg/kg, 7 mg/kg, 8 mg/kg, 9 mg/kg or 10 mg/kg.

Loxoribine and/or structurally related compounds are described in PCT International Patent Application WO 2019/226977, the contents of which are hereby incorporated by reference for all purposes and the specific purposes identified herein. In some embodiments, such compounds are represented by any one or more of the structures shown in Table 2. Any one of the compounds depicted in Table 2 is suitable for use in the methods of the present disclosure.

TABLE 2
Compound

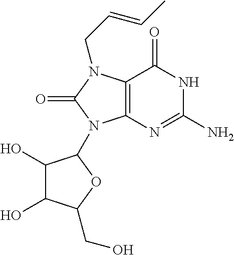 [SMILES: C/C=C/Cn1c(=O)n(C2OC(CO)C(O)C2O)c2nc(N)[nH]c(=O)c21]

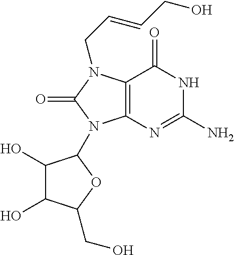 [SMILES: Nc1nc2c(c(=O)[nH]1)n(C/C=C/CO)c(=O)n2C1OC(CO)C(O)C1O]

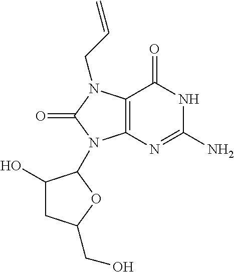 [SMILES: C=CCn1c(=O)n(C2OC(CO)CC2O)c2nc(N)[nH]c(=O)c21]

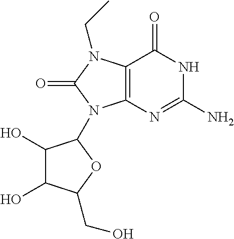 [SMILES: CCn1c(=O)n(C2OC(CO)C(O)C2O)c2nc(N)[nH]c(=O)c21]

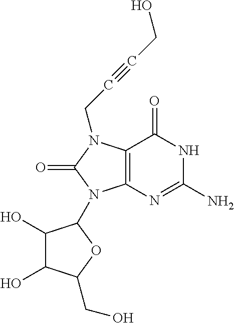 [SMILES: Nc1nc2c(c(=O)[nH]1)n(CC#CCO)c(=O)n2C1OC(CO)C(O)C1O]

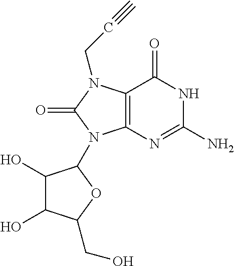 [SMILES: C#CCn1c(=O)n(C2OC(CO)C(O)C2O)c2nc(N)[nH]c(=O)c21]

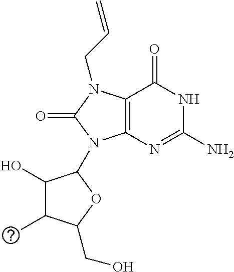 [SMILES: C=CCn1c(=O)n(C2OC(CO)C(C)C2O)c2nc(N)[nH]c(=O)c21]

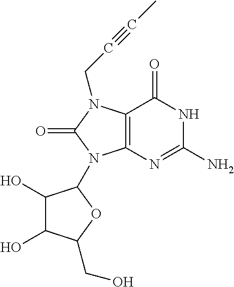 [SMILES: CC#CCn1c(=O)n(C2OC(CO)C(O)C2O)c2nc(N)[nH]c(=O)c21]

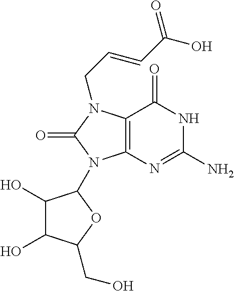 [SMILES: Nc1nc2c(c(=O)[nH]1)n(C/C=C/C(=O)O)c(=O)n2C1OC(CO)C(O)C1O]

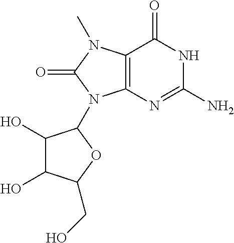 [SMILES: Cn1c(=O)n(C2OC(CO)C(O)C2O)c2nc(N)[nH]c(=O)c21]

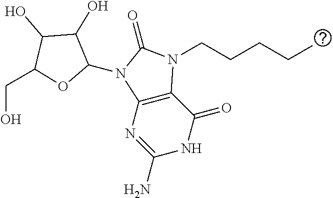 [SMILES: CCCCCn1c(=O)n(C2OC(CO)C(O)C2O)c2nc(N)[nH]c(=O)c21]

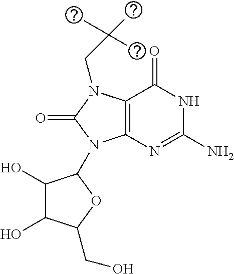 [SMILES: CC(C)(C)Cn1c(=O)n(C2OC(CO)C(O)C2O)c2nc(N)[nH]c(=O)c21]

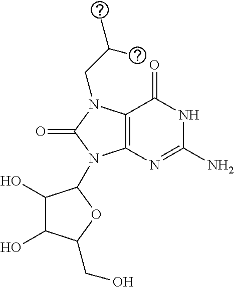 [SMILES: CC(C)Cn1c(=O)n(C2OC(CO)C(O)C2O)c2nc(N)[nH]c(=O)c21]

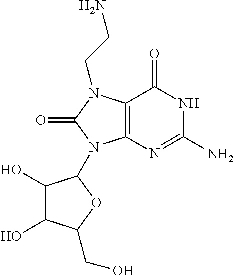 [SMILES: NCCn1c(=O)n(C2OC(CO)C(O)C2O)c2nc(N)[nH]c(=O)c21]

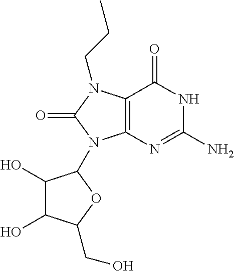 [SMILES: CCCn1c(=O)n(C2OC(CO)C(O)C2O)c2nc(N)[nH]c(=O)c21]

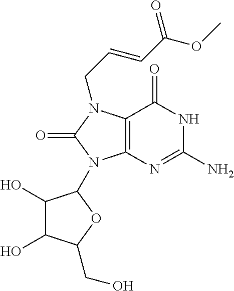 [SMILES: COC(=O)/C=C/Cn1c(=O)n(C2OC(CO)C(O)C2O)c2nc(N)[nH]c(=O)c21]

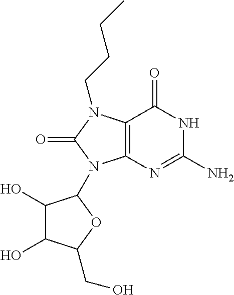 [SMILES: CCCCn1c(=O)n(C2OC(CO)C(O)C2O)c2nc(N)[nH]c(=O)c21]

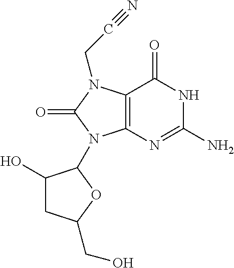 [SMILES: N#CCn1c(=O)n(C2OC(CO)CC2O)c2nc(N)[nH]c(=O)c21]

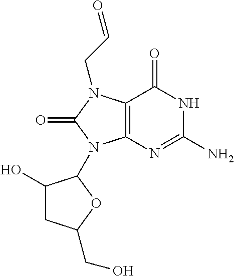 [SMILES: Nc1nc2c(c(=O)[nH]1)n(CC=O)c(=O)n2C1OC(CO)CC1O]

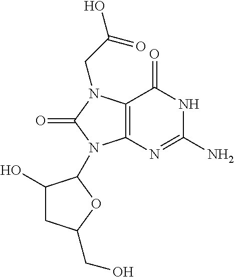 [SMILES: Nc1nc2c(c(=O)[nH]1)n(CC(=O)O)c(=O)n2C1OC(CO)CC1O]

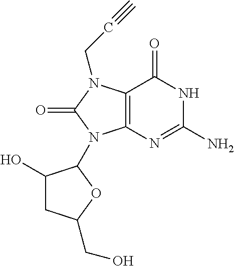 [SMILES: C#CCn1c(=O)n(C2OC(CO)CC2O)c2nc(N)[nH]c(=O)c21]

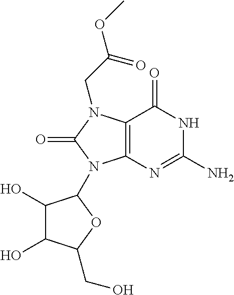 [SMILES: COC(=O)Cn1c(=O)n(C2OC(CO)C(O)C2O)c2nc(N)[nH]c(=O)c21]

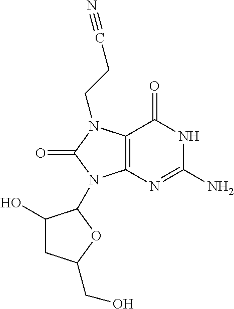 [SMILES: N#CCCn1c(=O)n(C2OC(CO)CC2O)c2nc(N)[nH]c(=O)c21]

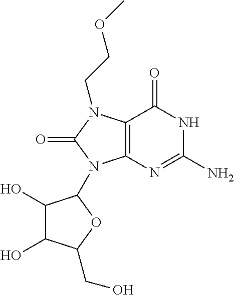 [SMILES: COCCn1c(=O)n(C2OC(CO)C(O)C2O)c2nc(N)[nH]c(=O)c21]

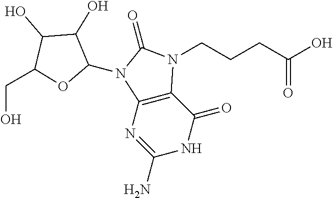 [SMILES: Nc1nc2c(c(=O)[nH]1)n(CCCC(=O)O)c(=O)n2C1OC(CO)C(O)C1O]

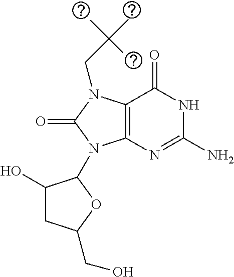 [SMILES: CC(C)(C)Cn1c(=O)n(C2OC(CO)CC2O)c2nc(N)[nH]c(=O)c21]

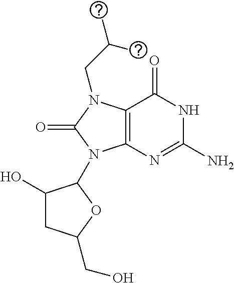 [SMILES: CC(C)Cn1c(=O)n(C2OC(CO)CC2O)c2nc(N)[nH]c(=O)c21]

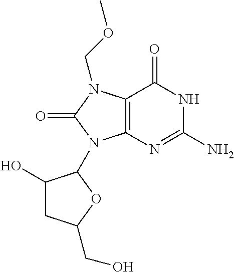 [SMILES: COCn1c(=O)n(C2OC(CO)CC2O)c2nc(N)[nH]c(=O)c21]

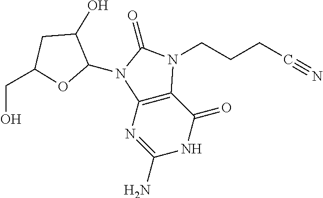 [SMILES: N#CCCCn1c(=O)n(C2OC(CO)CC2O)c2nc(N)[nH]c(=O)c21]

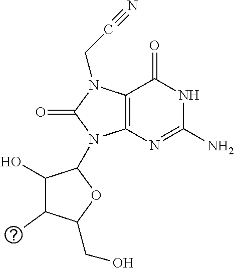 [SMILES: CC1C(CO)OC(n2c(=O)n(CC#N)c3c(=O)[nH]c(N)nc32)C1O]

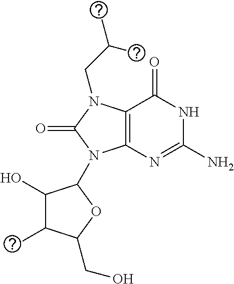 [SMILES: CC(C)Cn1c(=O)n(C2OC(CO)C(C)C2O)c2nc(N)[nH]c(=O)c21]

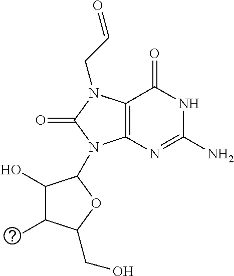 [SMILES: CC1C(CO)OC(n2c(=O)n(CC=O)c3c(=O)[nH]c(N)nc32)C1O]

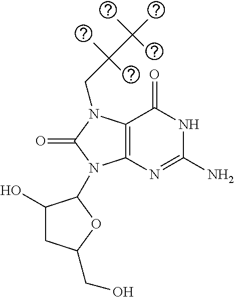 [SMILES: CC(C)(C)C(C)(C)Cn1c(=O)n(C2OC(CO)CC2O)c2nc(N)[nH]c(=O)c21]

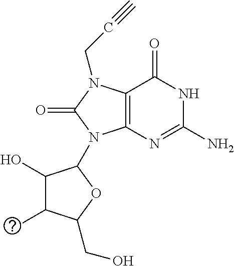 [SMILES: C#CCn1c(=O)n(C2OC(CO)C(C)C2O)c2nc(N)[nH]c(=O)c21]

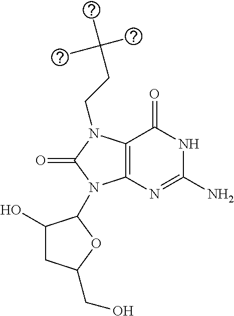 [SMILES: CC(C)(C)CCn1c(=O)n(C2OC(CO)CC2O)c2nc(N)[nH]c(=O)c21]

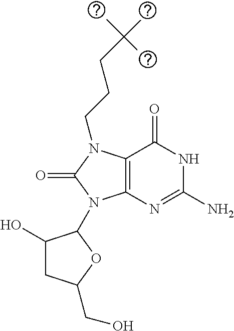 [SMILES: CC(C)(C)CCCn1c(=O)n(C2OC(CO)CC2O)c2nc(N)[nH]c(=O)c21]

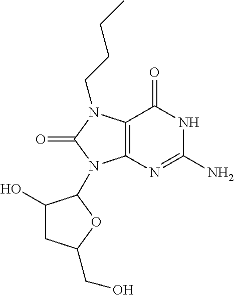 [SMILES: CCCCn1c(=O)n(C2OC(CO)CC2O)c2nc(N)[nH]c(=O)c21]

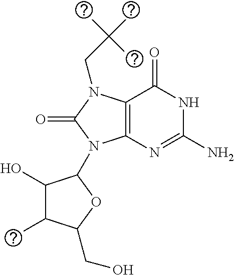 [SMILES: CC1C(CO)OC(n2c(=O)n(CC(C)(C)C)c3c(=O)[nH]c(N)nc32)C1O]

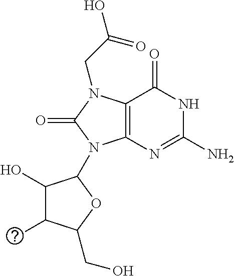 [SMILES: CC1C(CO)OC(n2c(=O)n(CC(=O)O)c3c(=O)[nH]c(N)nc32)C1O]

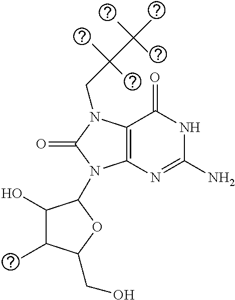 [SMILES: CC1C(CO)OC(n2c(=O)n(CC(C)(C)C(C)(C)C)c3c(=O)[nH]c(N)nc32)C1O]

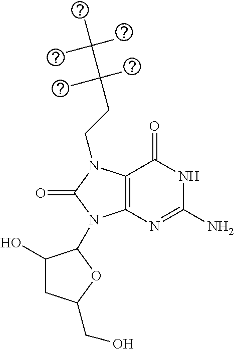 [SMILES: CC(C)(C)C(C)(C)CCn1c(=O)n(C2OC(CO)CC2O)c2nc(N)[nH]c(=O)c21]

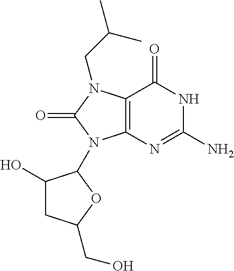 [SMILES: CC(C)Cn1c(=O)n(C2OC(CO)CC2O)c2nc(N)[nH]c(=O)c21]

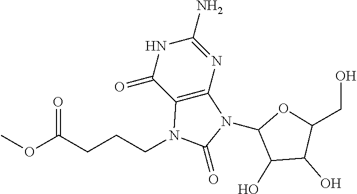 [SMILES: COC(=O)CCCn1c(=O)n(C2OC(CO)C(O)C2O)c2nc(N)[nH]c(=O)c21]

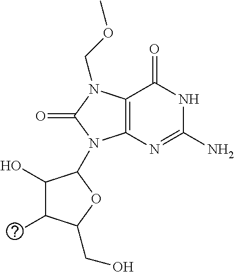 [SMILES: COCn1c(=O)n(C2OC(CO)C(C)C2O)c2nc(N)[nH]c(=O)c21]

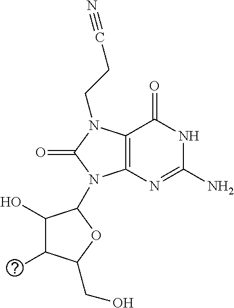 [SMILES: CC1C(CO)OC(n2c(=O)n(CCC#N)c3c(=O)[nH]c(N)nc32)C1O]

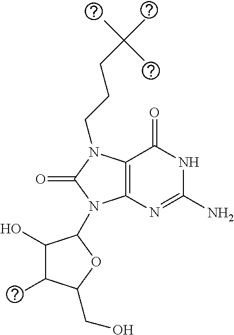 [SMILES: CC1C(CO)OC(n2c(=O)n(CCCC(C)(C)C)c3c(=O)[nH]c(N)nc32)C1O]

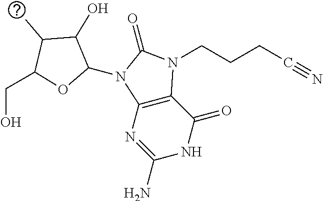 [SMILES: CC1C(CO)OC(n2c(=O)n(CCCC#N)c3c(=O)[nH]c(N)nc32)C1O]

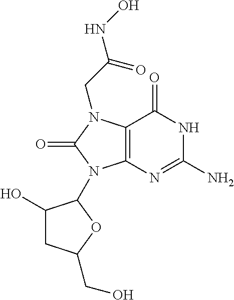 [SMILES: Nc1nc2c(c(=O)[nH]1)n(CC(=O)NO)c(=O)n2C1OC(CO)CC1O]

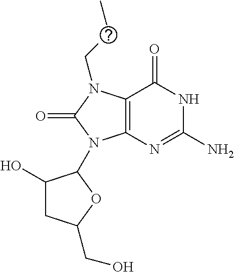 [SMILES: CCCn1c(=O)n(C2OC(CO)CC2O)c2nc(N)[nH]c(=O)c21]

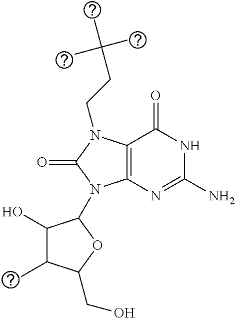 [SMILES: CC1C(CO)OC(n2c(=O)n(CCC(C)(C)C)c3c(=O)[nH]c(N)nc32)C1O]

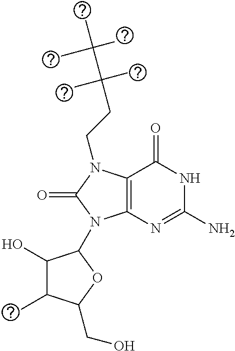 [SMILES: CC1C(CO)OC(n2c(=O)n(CCC(C)(C)C(C)(C)C)c3c(=O)[nH]c(N)nc32)C1O]

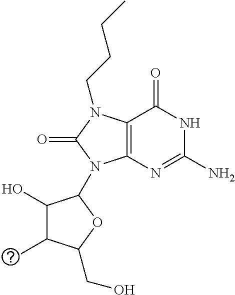 [SMILES: CCCCn1c(=O)n(C2OC(CO)C(C)C2O)c2nc(N)[nH]c(=O)c21]

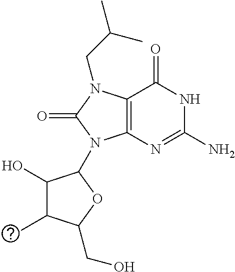 [SMILES: CC(C)Cn1c(=O)n(C2OC(CO)C(C)C2O)c2nc(N)[nH]c(=O)c21]

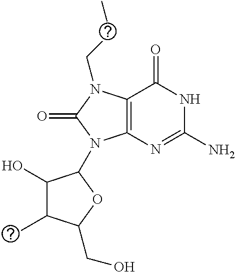 [SMILES: CCCn1c(=O)n(C2OC(CO)C(C)C2O)c2nc(N)[nH]c(=O)c21]

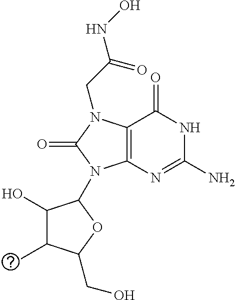 [SMILES: CC1C(CO)OC(n2c(=O)n(CC(=O)NO)c3c(=O)[nH]c(N)nc32)C1O]

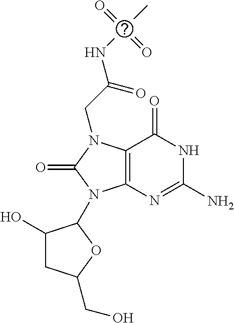

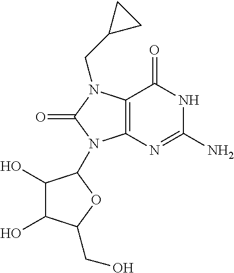 [SMILES: Nc1nc2c(c(=O)[nH]1)n(CC1CC1)c(=O)n2C1OC(CO)C(O)C1O]

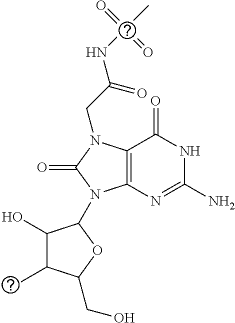

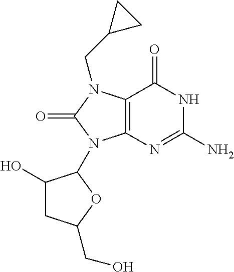 [SMILES: Nc1nc2c(c(=O)[nH]1)n(CC1CC1)c(=O)n2C1OC(CO)CC1O]

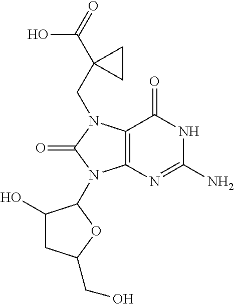 [SMILES: Nc1nc2c(c(=O)[nH]1)n(CC1(C(=O)O)CC1)c(=O)n2C1OC(CO)CC1O]

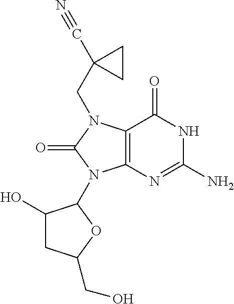 [SMILES: N#CC1(Cn2c(=O)n(C3OC(CO)CC3O)c3nc(N)[nH]c(=O)c32)CC1]

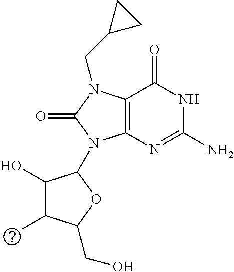 [SMILES: CC1C(CO)OC(n2c(=O)n(CC3CC3)c3c(=O)[nH]c(N)nc32)C1O]

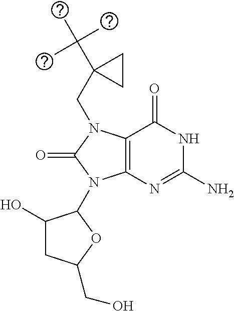 [SMILES: CC(C)(C)C1(Cn2c(=O)n(C3OC(CO)CC3O)c3nc(N)[nH]c(=O)c32)CC1]

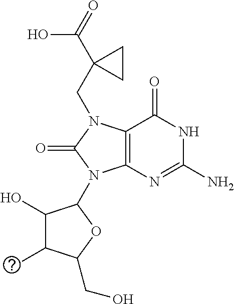 [SMILES: CC1C(CO)OC(n2c(=O)n(CC3(C(=O)O)CC3)c3c(=O)[nH]c(N)nc32)C1O]

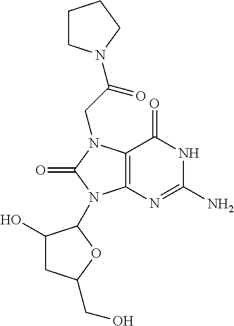 [SMILES: Nc1nc2c(c(=O)[nH]1)n(CC(=O)N1CCCC1)c(=O)n2C1OC(CO)CC1O]

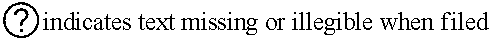

In some embodiments, the compound is Motolimod, a TLR-8 agonist which is chemically designated 2-amino-N,N-dipropyl-8-(4-(pyrrolidine-1-carbonyl)phenyl)-3H-benzo[b]azepine-4-carboxamide. Motolimod is currently in clinical trials for various cancers. Motolimod is represented by the structure:

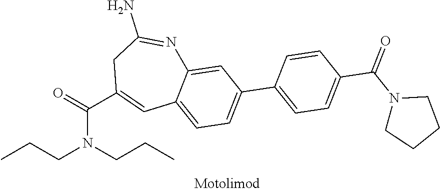 [SMILES: CCCN(CCC)C(=O)C1=Cc2ccc(-c3ccc(C(=O)N4CCCC4)cc3)cc2N=C(N)C1]

In some embodiments, motolimod is administered intravenously. In other embodiments, motolimod is administered subcutaneously.

Motolimod and/or structurally related compounds are described in PCT International Patent Applications WO 2011/022508 and WO 2011/022509, the contents of each of which are hereby incorporated by reference for all purposes and the specific purposes identified herein. In some embodiments, such compounds are represented by any one or more of the structures shown in Table 3. Any one of the compounds depicted in Table 3 is suitable for use in the methods of the present disclosure.

TABLE 3
Compound

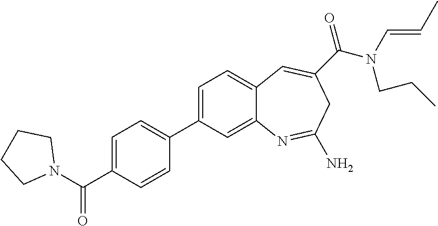 [SMILES: C/C=C/N(CCC)C(=O)C1=Cc2ccc(-c3ccc(C(=O)N4CCCC4)cc3)cc2N=C(N)C1]

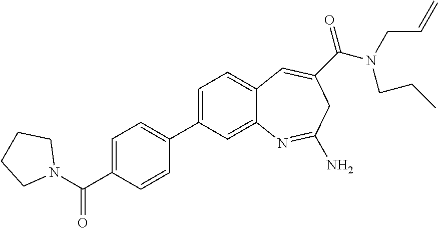 [SMILES: C=CCN(CCC)C(=O)C1=Cc2ccc(-c3ccc(C(=O)N4CCCC4)cc3)cc2N=C(N)C1]

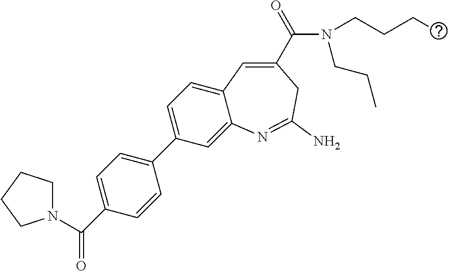 [SMILES: CCCCN(CCC)C(=O)C1=Cc2ccc(-c3ccc(C(=O)N4CCCC4)cc3)cc2N=C(N)C1]

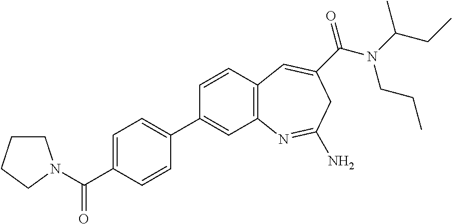 [SMILES: CCCN(C(=O)C1=Cc2ccc(-c3ccc(C(=O)N4CCCC4)cc3)cc2N=C(N)C1)C(C)CC]

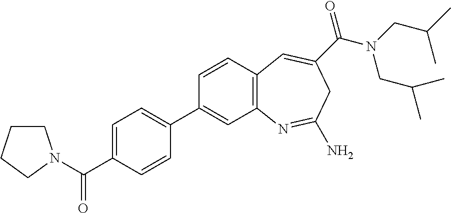 [SMILES: CC(C)CN(CC(C)C)C(=O)C1=Cc2ccc(-c3ccc(C(=O)N4CCCC4)cc3)cc2N=C(N)C1]

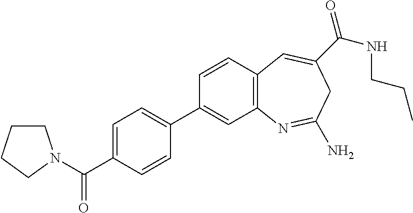 [SMILES: CCCNC(=O)C1=Cc2ccc(-c3ccc(C(=O)N4CCCC4)cc3)cc2N=C(N)C1]

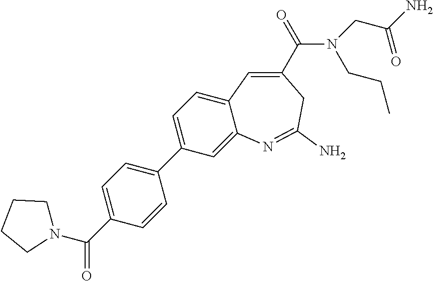 [SMILES: CCCN(CC(N)=O)C(=O)C1=Cc2ccc(-c3ccc(C(=O)N4CCCC4)cc3)cc2N=C(N)C1]

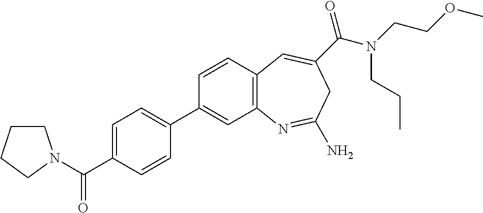 [SMILES: CCCN(CCOC)C(=O)C1=Cc2ccc(-c3ccc(C(=O)N4CCCC4)cc3)cc2N=C(N)C1]

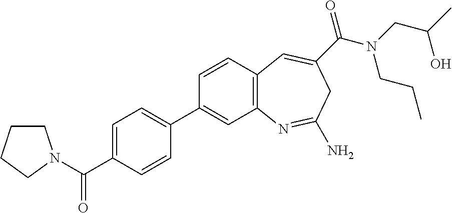 [SMILES: CCCN(CC(C)O)C(=O)C1=Cc2ccc(-c3ccc(C(=O)N4CCCC4)cc3)cc2N=C(N)C1]

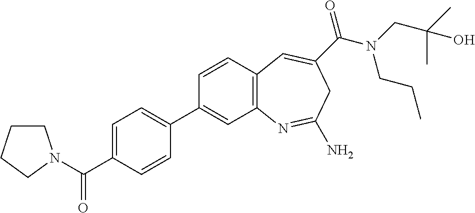 [SMILES: CCCN(CC(C)(C)O)C(=O)C1=Cc2ccc(-c3ccc(C(=O)N4CCCC4)cc3)cc2N=C(N)C1]

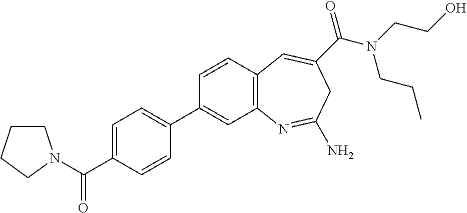 [SMILES: CCCN(CCO)C(=O)C1=Cc2ccc(-c3ccc(C(=O)N4CCCC4)cc3)cc2N=C(N)C1]

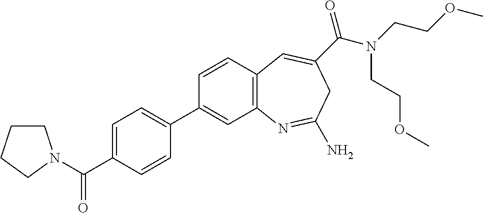 [SMILES: COCCN(CCOC)C(=O)C1=Cc2ccc(-c3ccc(C(=O)N4CCCC4)cc3)cc2N=C(N)C1]

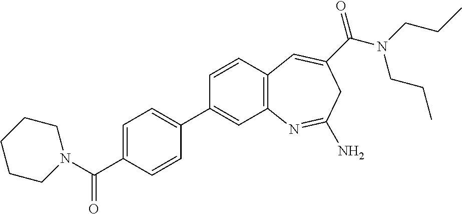 [SMILES: CCCN(CCC)C(=O)C1=Cc2ccc(-c3ccc(C(=O)N4CCCCC4)cc3)cc2N=C(N)C1]

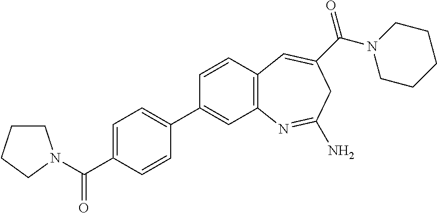 [SMILES: NC1=Nc2cc(-c3ccc(C(=O)N4CCCC4)cc3)ccc2C=C(C(=O)N2CCCCC2)C1]

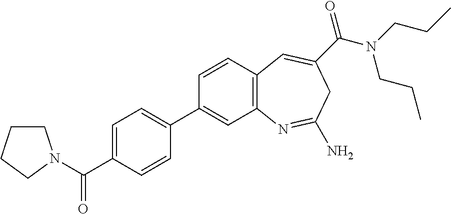 [SMILES: CCCN(CCC)C(=O)C1=Cc2ccc(-c3ccc(C(=O)N4CCCC4)cc3)cc2N=C(N)C1]

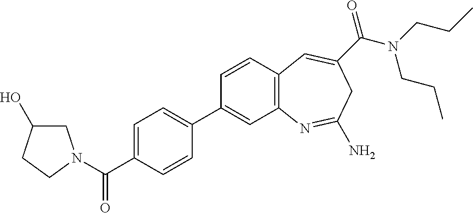 [SMILES: CCCN(CCC)C(=O)C1=Cc2ccc(-c3ccc(C(=O)N4CCC(O)C4)cc3)cc2N=C(N)C1]

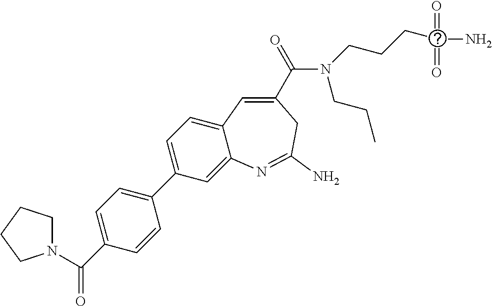

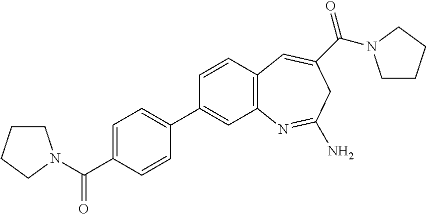 [SMILES: NC1=Nc2cc(-c3ccc(C(=O)N4CCCC4)cc3)ccc2C=C(C(=O)N2CCCC2)C1]

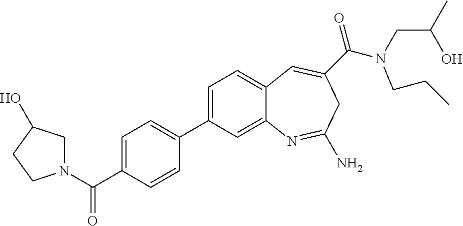 [SMILES: CCCN(CC(C)O)C(=O)C1=Cc2ccc(-c3ccc(C(=O)N4CCC(O)C4)cc3)cc2N=C(N)C1]

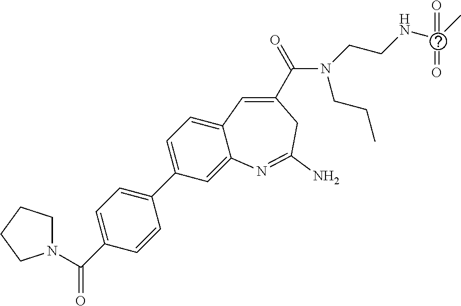

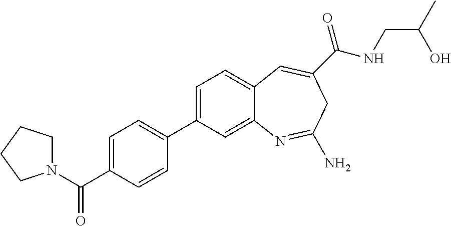 [SMILES: CC(O)CNC(=O)C1=Cc2ccc(-c3ccc(C(=O)N4CCCC4)cc3)cc2N=C(N)C1]

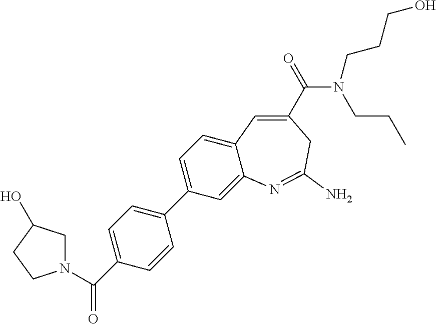 [SMILES: CCCN(CCCO)C(=O)C1=Cc2ccc(-c3ccc(C(=O)N4CCC(O)C4)cc3)cc2N=C(N)C1]

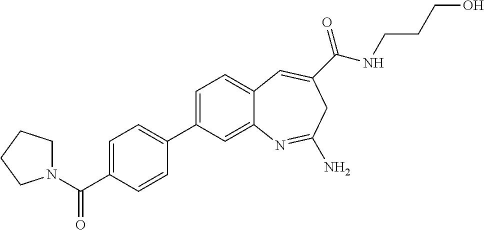 [SMILES: NC1=Nc2cc(-c3ccc(C(=O)N4CCCC4)cc3)ccc2C=C(C(=O)NCCCO)C1]

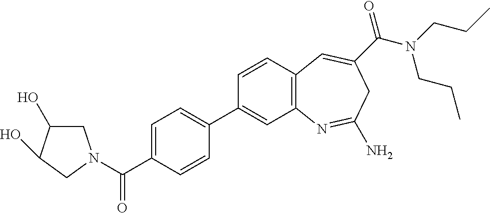 [SMILES: CCCN(CCC)C(=O)C1=Cc2ccc(-c3ccc(C(=O)N4CC(O)C(O)C4)cc3)cc2N=C(N)C1]

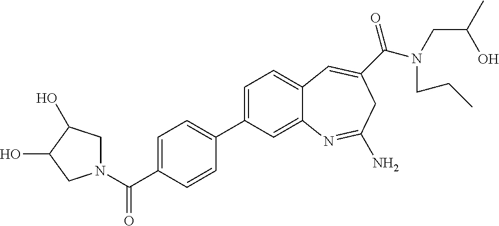 [SMILES: CCCN(CC(C)O)C(=O)C1=Cc2ccc(-c3ccc(C(=O)N4CC(O)C(O)C4)cc3)cc2N=C(N)C1]

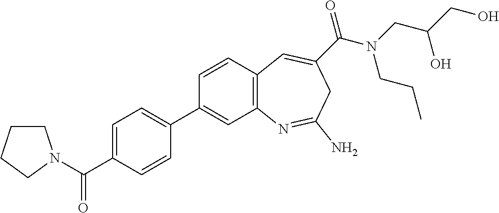 [SMILES: CCCN(CC(O)CO)C(=O)C1=Cc2ccc(-c3ccc(C(=O)N4CCCC4)cc3)cc2N=C(N)C1]

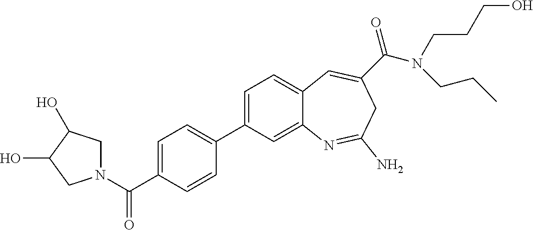 [SMILES: CCCN(CCCO)C(=O)C1=Cc2ccc(-c3ccc(C(=O)N4CC(O)C(O)C4)cc3)cc2N=C(N)C1]

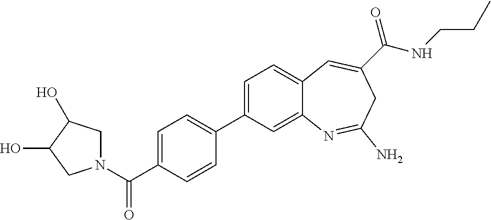 [SMILES: CCCNC(=O)C1=Cc2ccc(-c3ccc(C(=O)N4CC(O)C(O)C4)cc3)cc2N=C(N)C1]

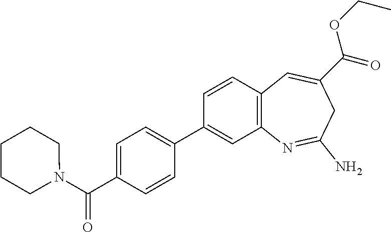 [SMILES: CCOC(=O)C1=Cc2ccc(-c3ccc(C(=O)N4CCCCC4)cc3)cc2N=C(N)C1]

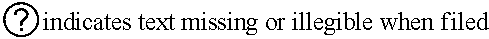

In some embodiments, the compound is LHC-165, a TLR-7 agonist which is chemically designated 3-[5-amino-2-[4-[2-[4-[2-(3,3-difluoro-3-phosphonopropoxy)ethoxy]-2-methylphenyl]ethyl]benzo[f][1,7]naphthyridin-8-yl]propanoic acid. LHC-165 is currently in clinical trials for the treatment of solid tumors. LHC-165 is represented by the structure:

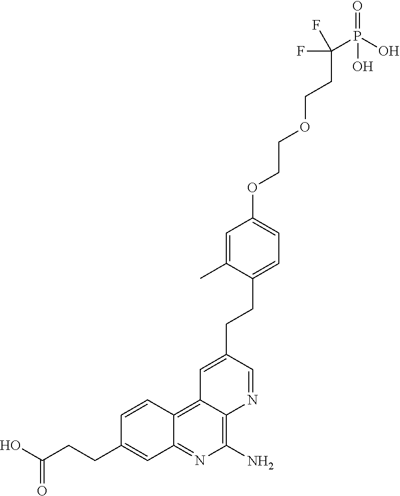 [SMILES: Cc1cc(OCCOCCC(F)(F)P(=O)(O)O)ccc1CCc1cnc2c(N)nc3cc(CCC(=O)O)ccc3c2c1]

In some embodiments, LHC-165 is administered intravenously. In other embodiments, LHC-165 is administered subcutaneously.

LHC-165 and/or structurally related compounds are described in PCT International Patent Applications WO 2011/130379, WO 2011/049677 and WO 2009/111337, the contents of each of which are hereby incorporated by reference for all purposes and the specific purposes identified herein. In some embodiments, such compounds are represented by any one or more of the structures shown in Table 4. Any one of the compounds depicted in Table 4 is suitable for use in the methods of the present disclosure.

TABLE 4
Compound

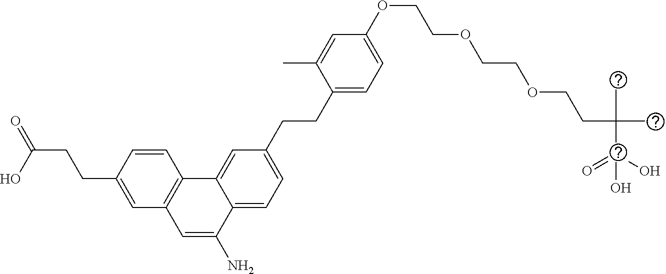

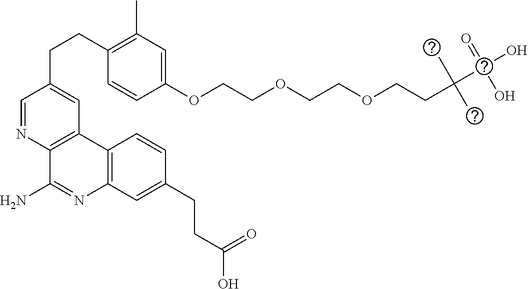

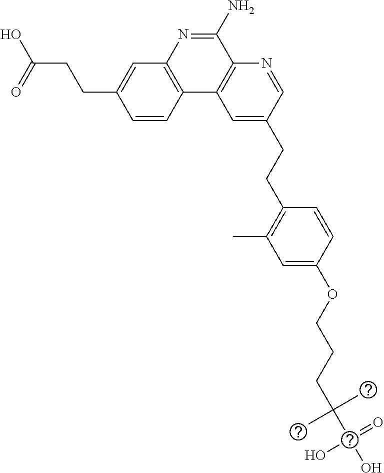

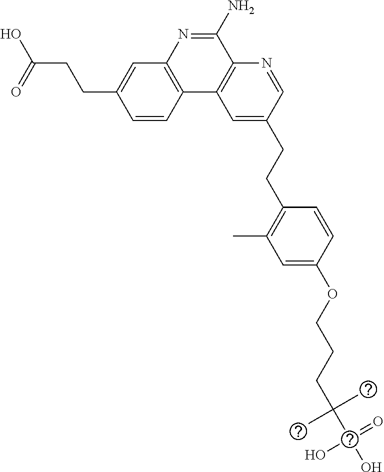

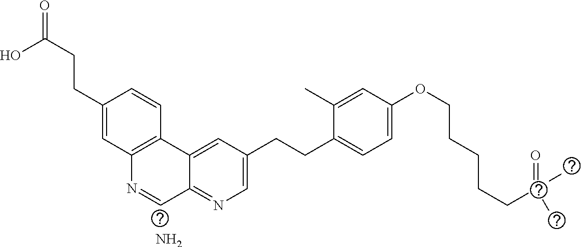

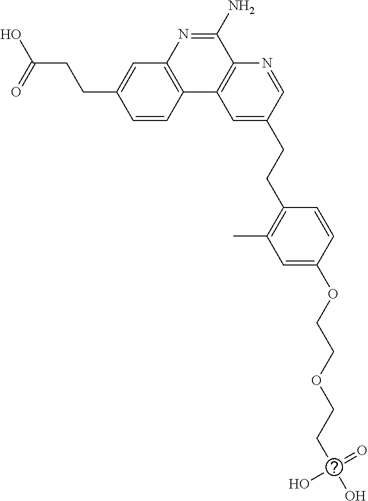

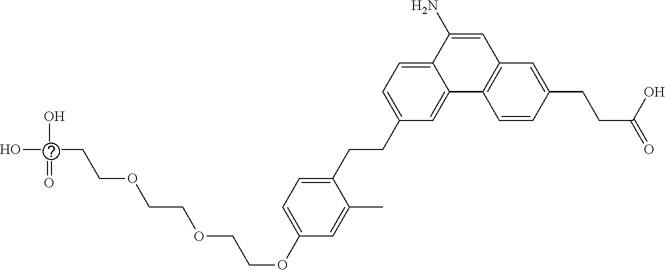

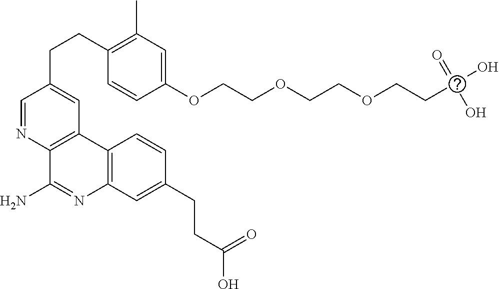

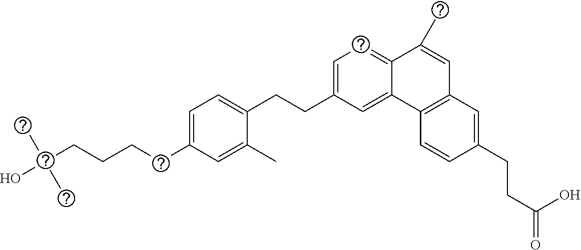 [SMILES: Cc1cc(CCCCC(C)(C)O)ccc1CCc1ccc2c(C)cc3cc(CCC(=O)O)ccc3c2c1]

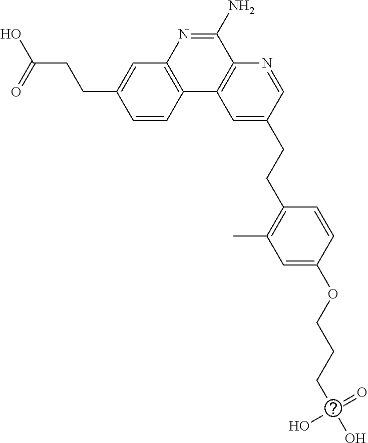

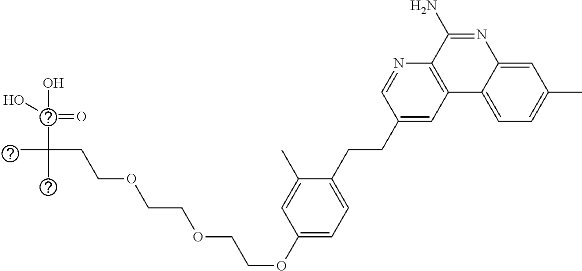

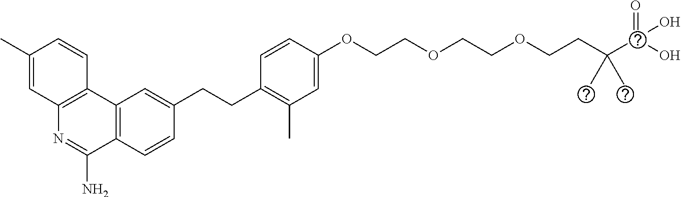

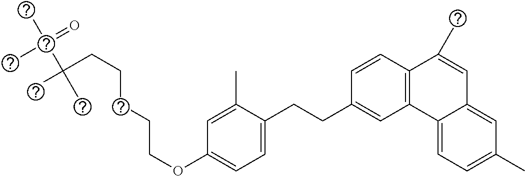

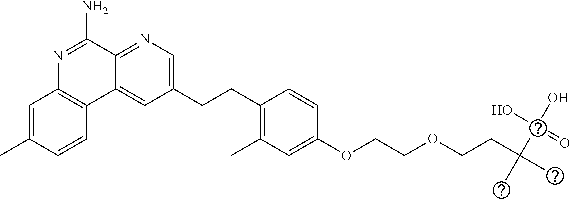

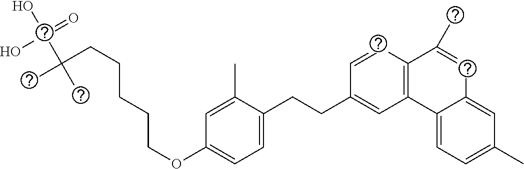

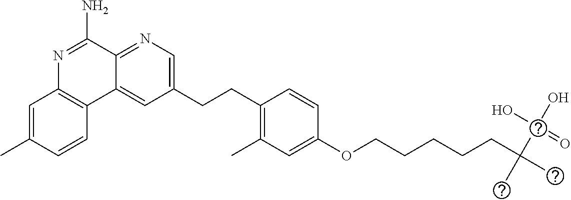

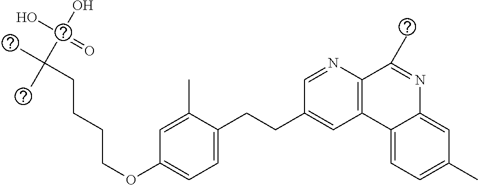

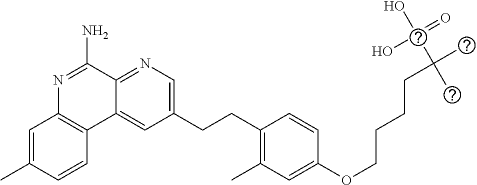

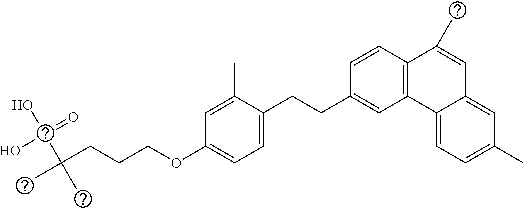

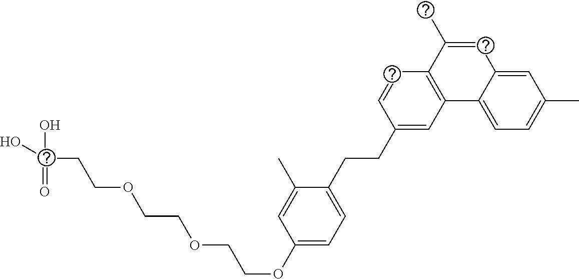

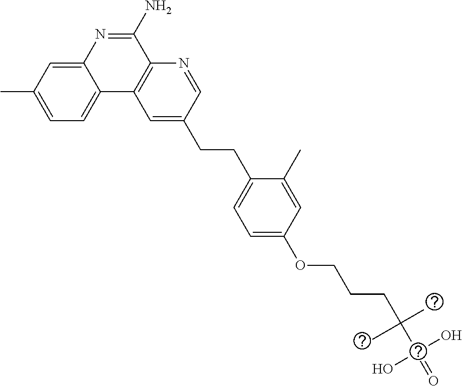

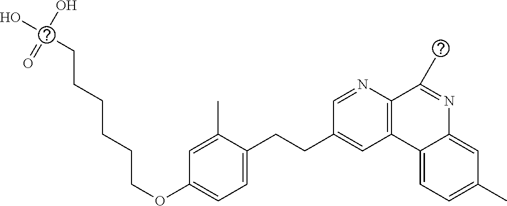

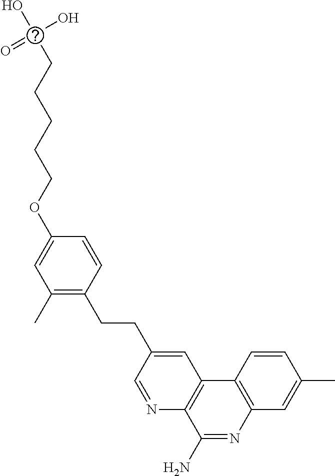

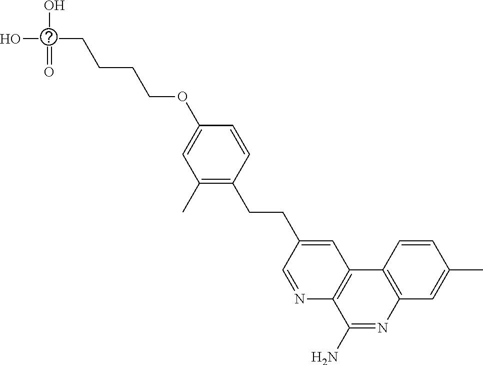

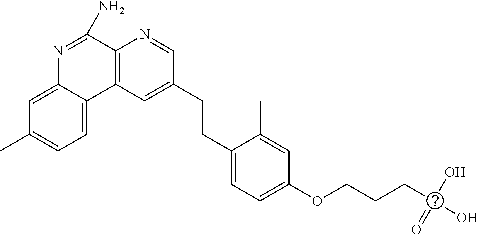

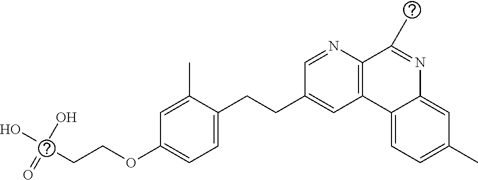

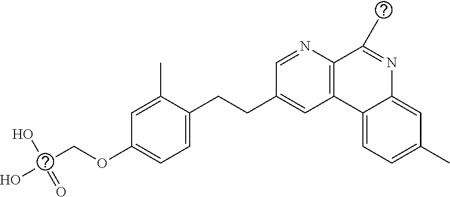

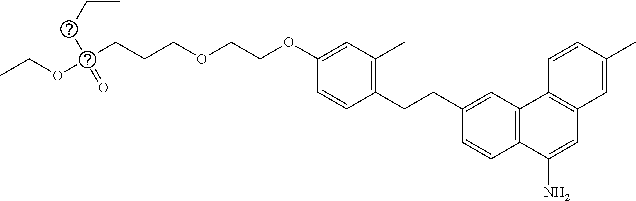

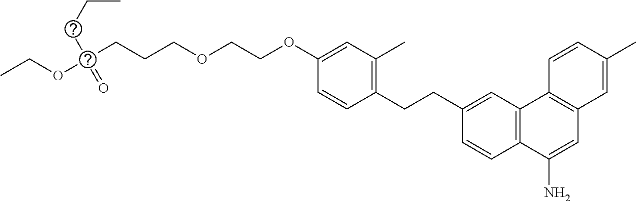

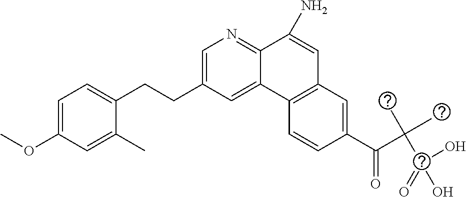

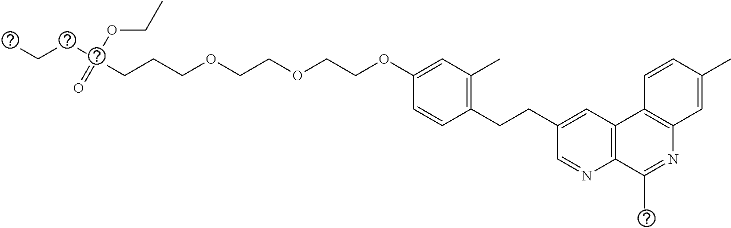

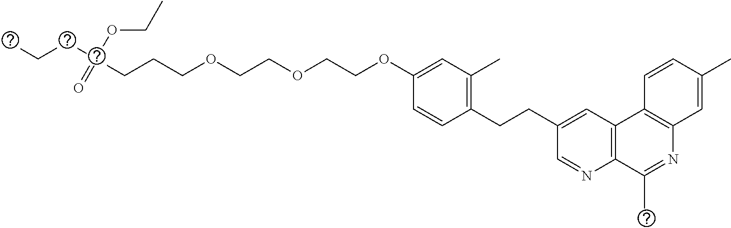

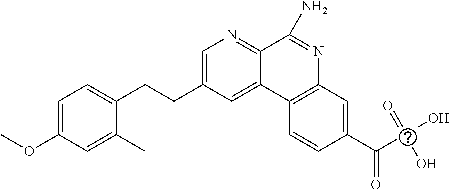

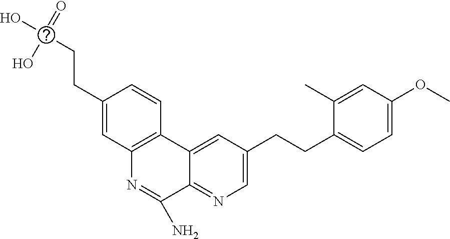

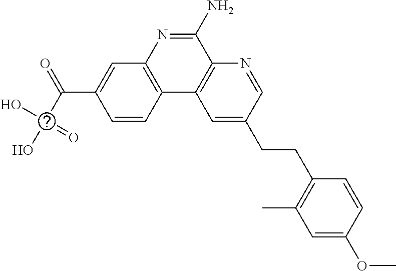

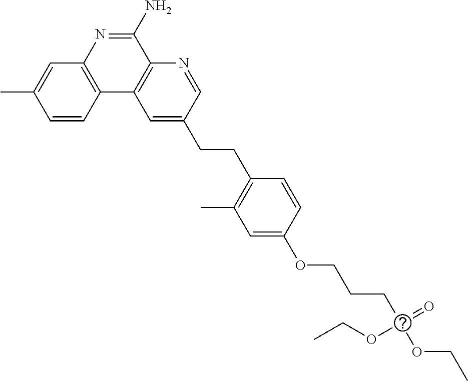

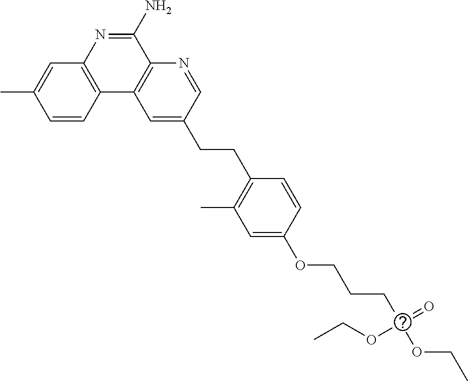

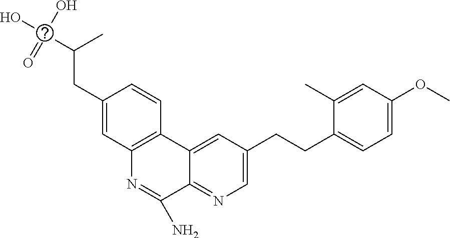

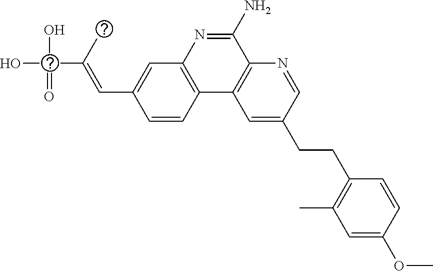

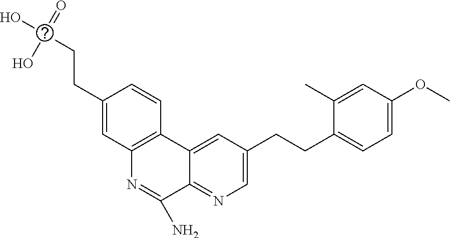

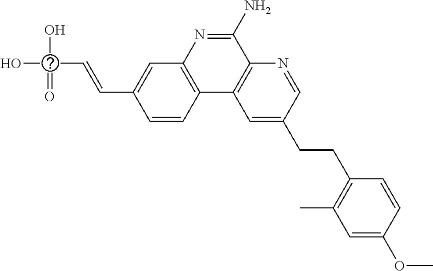

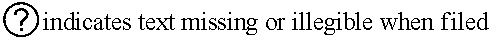

In some embodiments, the compound is Resiquimod, a TLR-7/TLR-8 agonist which is chemically designated 1-(4-amino-2-(ethoxymethyl)-1H-imidazo[4,5-c]quinolin-1-yl)-2-methylpropan-2-ol. Resiquimod is currently in clinical trials for the treatment of cutaneous T-cell lymphoma (CTCL), melanoma, actinic keratosis and warts. Resiquimod is represented by the structure:

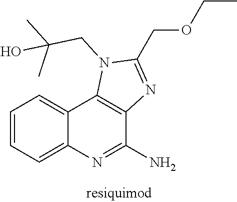 [SMILES: CCOCc1nc2c(N)nc3ccccc3c2n1CC(C)(C)O]

In some embodiment, resiquimod is administered topically. In other embodiments, resiquimod is administered topically as a gel. In other embodiments, resiquimod is administered topically as a gel comprising between 0.01% and about 1% resiquimod.

Resiquimod and/or structurally related compounds are described in Patent Applications WO2019/095455, WO202/0051356, WO2015/162075, CN108299421, and WO2020/023680, the contents of each of which are hereby incorporated by reference for all purposes and the specific purposes identified herein. In some embodiments, such compounds are represented by any one or more of the structures shown in Table 5. Any one of the compounds depicted in Table 5 is suitable for use in the methods of the present disclosure.

TABLE 5
Compound

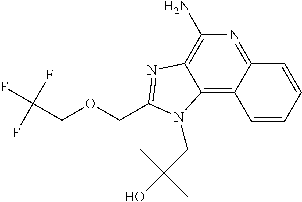 [SMILES: CC(C)(O)Cn1c(COCC(F)(F)F)nc2c(N)nc3ccccc3c21]

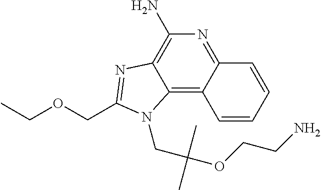 [SMILES: CCOCc1nc2c(N)nc3ccccc3c2n1CC(C)(C)OCCN]

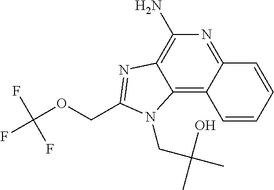 [SMILES: CC(C)(O)Cn1c(COC(F)(F)F)nc2c(N)nc3ccccc3c21]

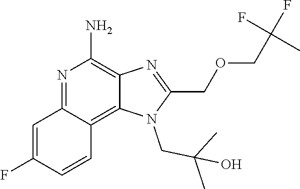 [SMILES: CC(C)(O)Cn1c(COCC(C)(F)F)nc2c(N)nc3cc(F)ccc3c21]

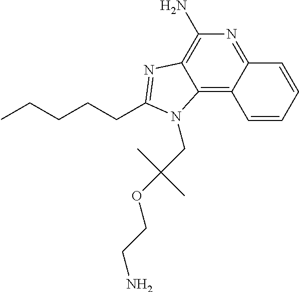 [SMILES: CCCCCc1nc2c(N)nc3ccccc3c2n1CC(C)(C)OCCN]

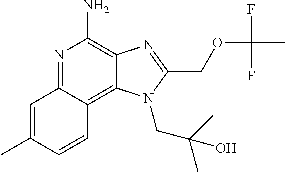 [SMILES: Cc1ccc2c(c1)nc(N)c1nc(COC(C)(F)F)n(CC(C)(C)O)c12]

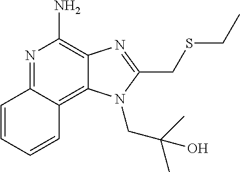 [SMILES: CCSCc1nc2c(N)nc3ccccc3c2n1CC(C)(C)O]

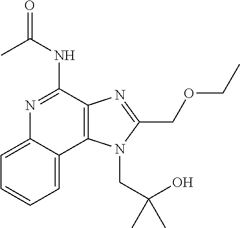 [SMILES: CCOCc1nc2c(NC(C)=O)nc3ccccc3c2n1CC(C)(C)O]

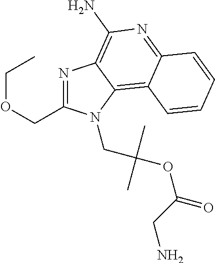 [SMILES: CCOCc1nc2c(N)nc3ccccc3c2n1CC(C)(C)OC(=O)CN]

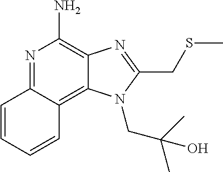 [SMILES: CSCc1nc2c(N)nc3ccccc3c2n1CC(C)(C)O]

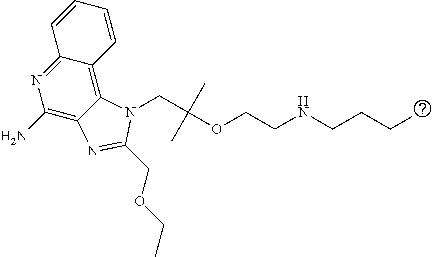 [SMILES: CCCCNCCOC(C)(C)Cn1c(COCC)nc2c(N)nc3ccccc3c21]

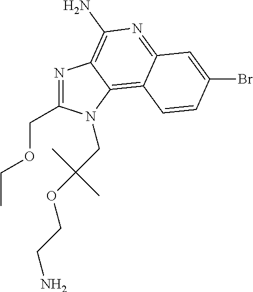 [SMILES: CCOCc1nc2c(N)nc3cc(Br)ccc3c2n1CC(C)(C)OCCN]

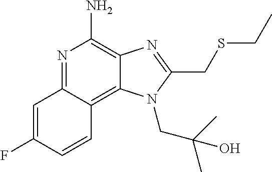 [SMILES: CCSCc1nc2c(N)nc3cc(F)ccc3c2n1CC(C)(C)O]

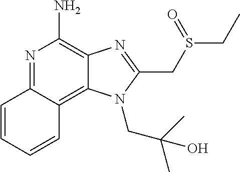 [SMILES: CCS(=O)Cc1nc2c(N)nc3ccccc3c2n1CC(C)(C)O]

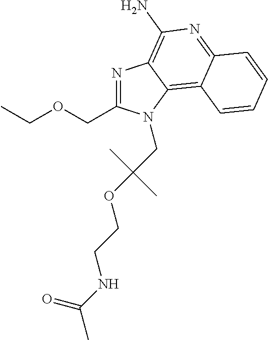 [SMILES: CCOCc1nc2c(N)nc3ccccc3c2n1CC(C)(C)OCCNC(C)=O]

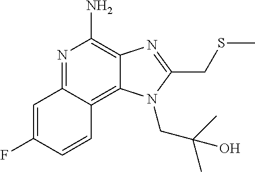 [SMILES: CSCc1nc2c(N)nc3cc(F)ccc3c2n1CC(C)(C)O]

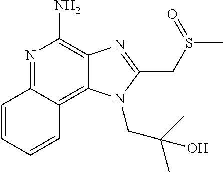 [SMILES: CS(=O)Cc1nc2c(N)nc3ccccc3c2n1CC(C)(C)O]

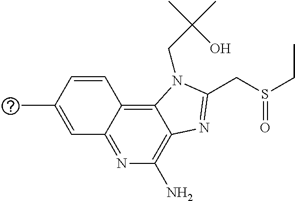 [SMILES: CCS(=O)Cc1nc2c(N)nc3cc(C)ccc3c2n1CC(C)(C)O]

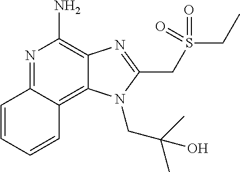 [SMILES: CCS(=O)(=O)Cc1nc2c(N)nc3ccccc3c2n1CC(C)(C)O]

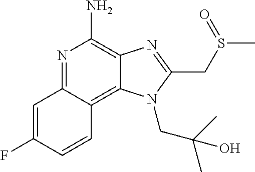 [SMILES: CS(=O)Cc1nc2c(N)nc3cc(F)ccc3c2n1CC(C)(C)O]

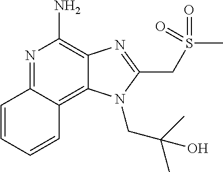 [SMILES: CC(C)(O)Cn1c(CS(C)(=O)=O)nc2c(N)nc3ccccc3c21]

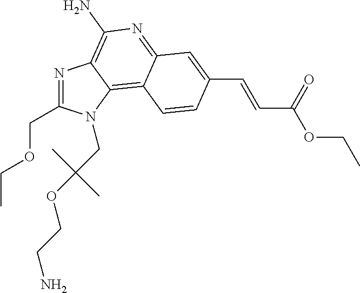 [SMILES: CCOCc1nc2c(N)nc3cc(/C=C/C(=O)OCC)ccc3c2n1CC(C)(C)OCCN]

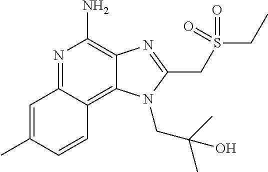 [SMILES: CCS(=O)(=O)Cc1nc2c(N)nc3cc(C)ccc3c2n1CC(C)(C)O]

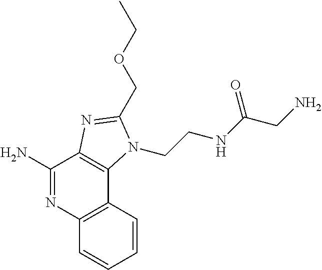 [SMILES: CCOCc1nc2c(N)nc3ccccc3c2n1CCNC(=O)CN]

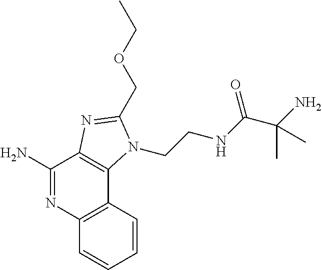 [SMILES: CCOCc1nc2c(N)nc3ccccc3c2n1CCNC(=O)C(C)(C)N]

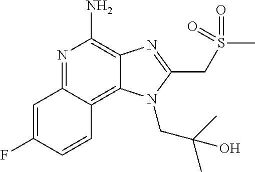 [SMILES: CC(C)(O)Cn1c(CS(C)(=O)=O)nc2c(N)nc3cc(F)ccc3c21]

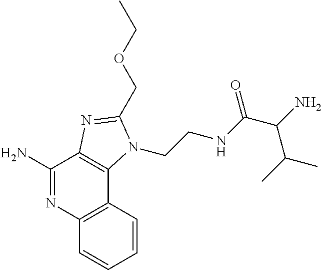 [SMILES: CCOCc1nc2c(N)nc3ccccc3c2n1CCNC(=O)C(N)C(C)C]

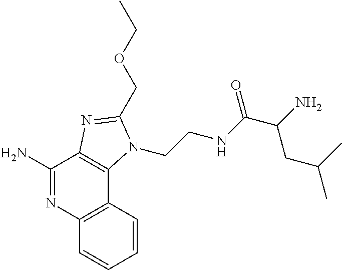 [SMILES: CCOCc1nc2c(N)nc3ccccc3c2n1CCNC(=O)C(N)CC(C)C]

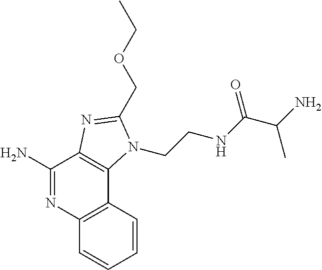 [SMILES: CCOCc1nc2c(N)nc3ccccc3c2n1CCNC(=O)C(C)N]

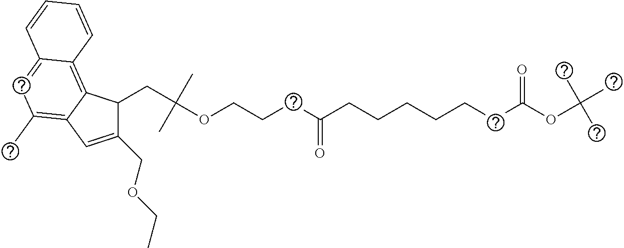 [SMILES: CCOCC1=Cc2c(C)cc3ccccc3c2C1CC(C)(C)OCCCC(=O)CCCCCCC(=O)OC(C)(C)C]

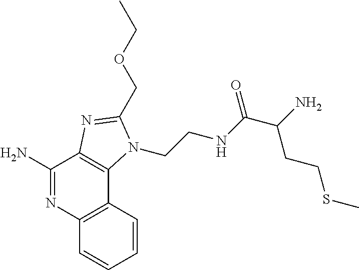 [SMILES: CCOCc1nc2c(N)nc3ccccc3c2n1CCNC(=O)C(N)CCSC]

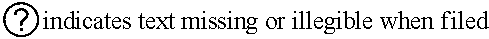

In some embodiments, the compound is GSK-2245035, is a TLR-7 agonist which is chemically designated 6-amino-2-(pentan-2-yloxy)-9-(5-(piperidin-1-yl)pentyl)-7,9-dihydro-8H-purin-8-one. GSK-2245035 was previously studied in clinical trials for the treatment of asthma and allergic rhinitis. GSK-2245035 is represented by the structure:

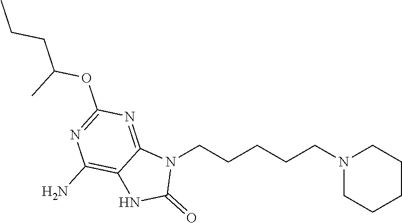 [SMILES: CCCC(C)Oc1nc(N)c2[nH]c(=O)n(CCCCCN3CCCCC3)c2n1]

In some embodiments, GSK-2245035 is administered as a nasal spray. In some embodiments, GSK-2245035 is administered as a nasal spray at a dose of 0.2 microgram (mcg)/mL delivering 10 ng GSK2245035 per actuation. In some embodiment, GSK-2245035 is formulated in in saline. In some embodiments, GSK-2245035 is formulated in saline preserved with Benzalkonium Chloride and Disodium Edetate.

GSK-2245035 and/or structurally related compounds are described in PCT International Patent Application WO 2007/034881, the contents of which are hereby incorporated by reference for all purposes and the specific purposes identified herein. In some embodiments, such compounds are represented by any one or more of the structures shown in Table 6. Any one of the compounds depicted in Table 6 is suitable for use in the methods of the present disclosure.

TABLE 6
Compound

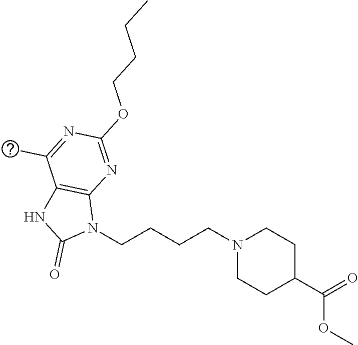

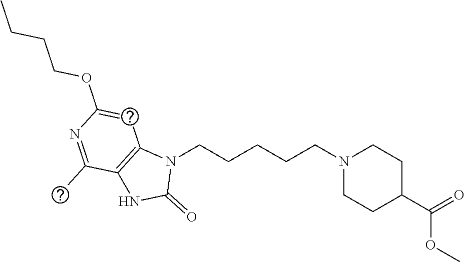 [SMILES: CCCCOc1cc2c([nH]c(=O)n2CCCCCN2CCC(C(=O)OC)CC2)c(C)n1]

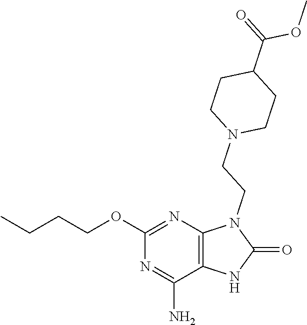 [SMILES: CCCCOc1nc(N)c2[nH]c(=O)n(CCN3CCC(C(=O)OC)CC3)c2n1]

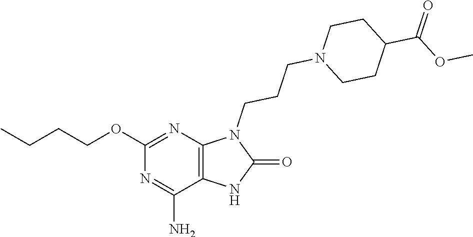 [SMILES: CCCCOc1nc(N)c2[nH]c(=O)n(CCCN3CCC(C(=O)OC)CC3)c2n1]

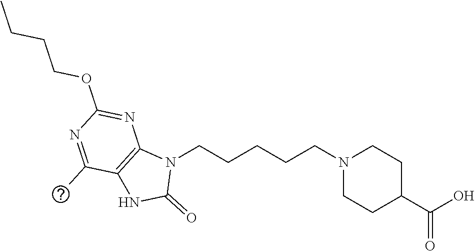 [SMILES: CCCCOc1nc(C)c2[nH]c(=O)n(CCCCCN3CCC(C(=O)O)CC3)c2n1]

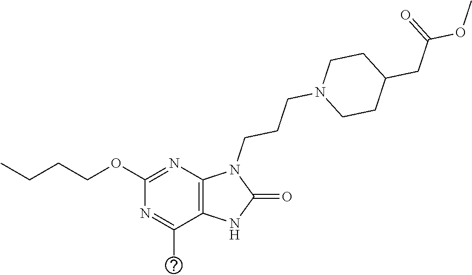 [SMILES: CCCCOc1nc(C)c2[nH]c(=O)n(CCCN3CCC(CC(=O)OC)CC3)c2n1]

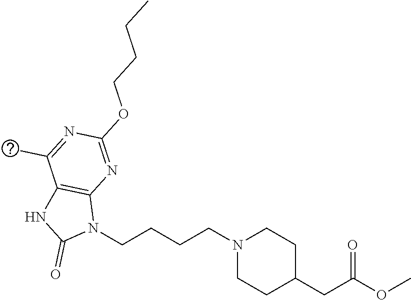

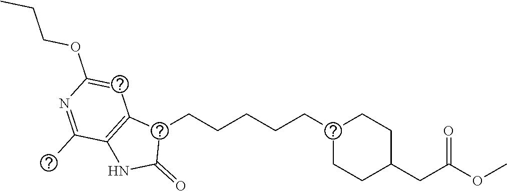 [SMILES: CCCOc1cc2c(c(C)n1)NC(=O)C2CCCCCC1CCC(CC(=O)OC)CC1]

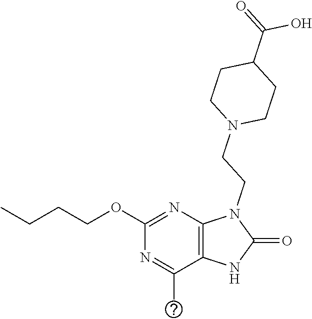 [SMILES: CCCCOc1nc(C)c2[nH]c(=O)n(CCN3CCC(C(=O)O)CC3)c2n1]

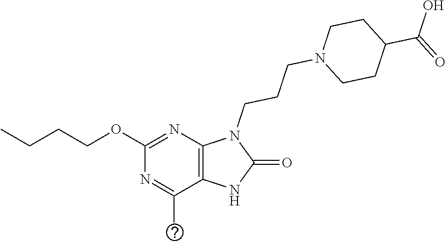 [SMILES: CCCCOc1nc(C)c2[nH]c(=O)n(CCCN3CCC(C(=O)O)CC3)c2n1]

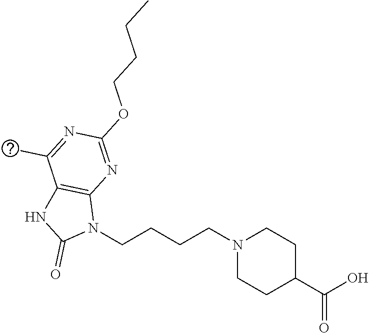 [SMILES: CCCCOc1nc(C)c2[nH]c(=O)n(CCCCN3CCC(C(=O)O)CC3)c2n1]

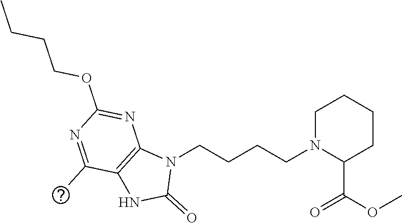 [SMILES: CCCCOc1nc(C)c2[nH]c(=O)n(CCCCN3CCCCC3C(=O)OC)c2n1]

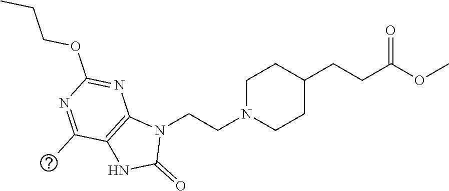 [SMILES: CCCOc1nc(C)c2[nH]c(=O)n(CCN3CCC(CCC(=O)OC)CC3)c2n1]

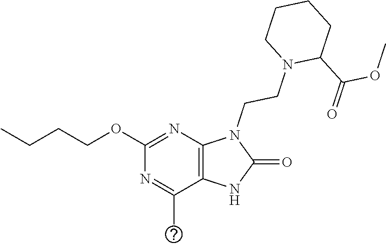 [SMILES: CCCCOc1nc(C)c2[nH]c(=O)n(CCN3CCCCC3C(=O)OC)c2n1]

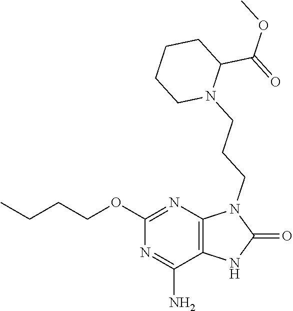 [SMILES: CCCCOc1nc(N)c2[nH]c(=O)n(CCCN3CCCCC3C(=O)OC)c2n1]

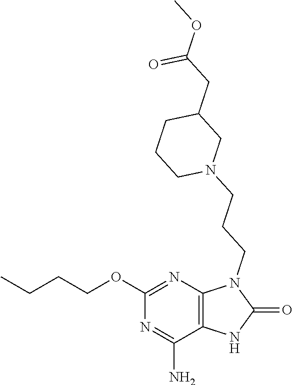 [SMILES: CCCCOc1nc(N)c2[nH]c(=O)n(CCCN3CCCC(CC(=O)OC)C3)c2n1]

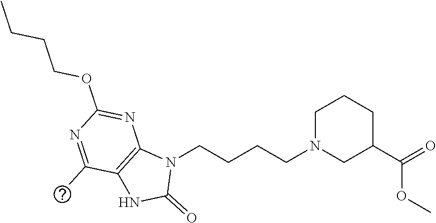 [SMILES: CCCCOc1nc(C)c2[nH]c(=O)n(CCCCN3CCCC(C(=O)OC)C3)c2n1]

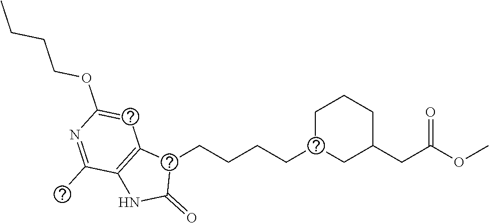 [SMILES: CCCCOc1cc2c(c(C)n1)NC(=O)C2CCCCC1CCCC(CC(=O)OC)C1]

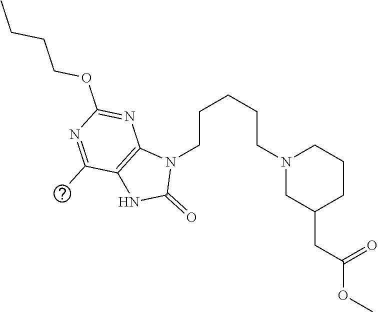 [SMILES: CCCCOc1nc(C)c2[nH]c(=O)n(CCCCCN3CCCC(CC(=O)OC)C3)c2n1]

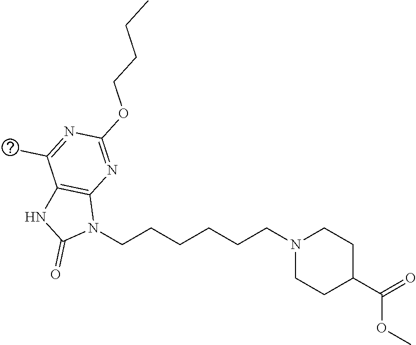

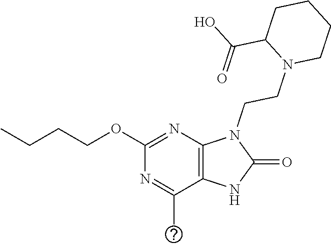 [SMILES: CCCCOc1nc(C)c2[nH]c(=O)n(CCN3CCCCC3C(=O)O)c2n1]

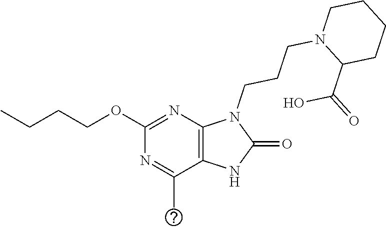 [SMILES: CCCCOc1nc(C)c2[nH]c(=O)n(CCCN3CCCCC3C(=O)O)c2n1]

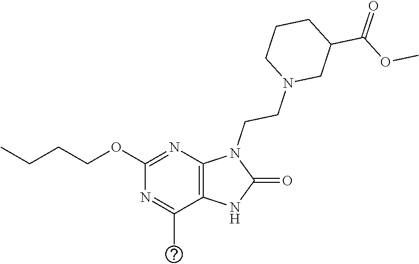 [SMILES: CCCCOc1nc(C)c2[nH]c(=O)n(CCN3CCCC(C(=O)OC)C3)c2n1]

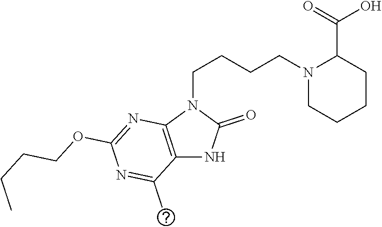 [SMILES: CCCCOc1nc(C)c2[nH]c(=O)n(CCCCN3CCCCC3C(=O)O)c2n1]

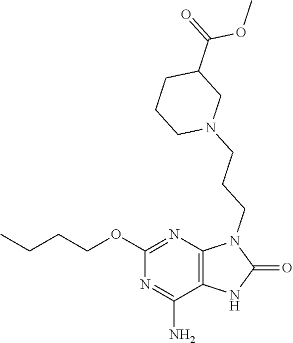 [SMILES: CCCCOc1nc(N)c2[nH]c(=O)n(CCCN3CCCC(C(=O)OC)C3)c2n1]

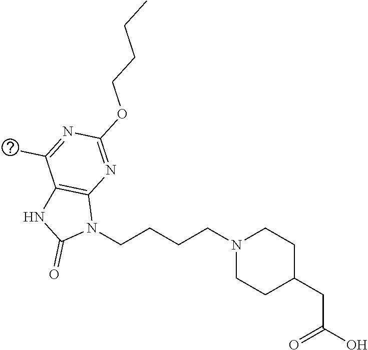 [SMILES: CCCCOc1nc(C)c2[nH]c(=O)n(CCCCN3CCC(CC(=O)O)CC3)c2n1]

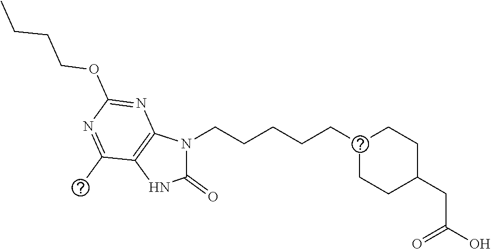 [SMILES: CCCCOc1nc(C)c2[nH]c(=O)n(CCCCCC3CCC(CC(=O)O)CC3)c2n1]

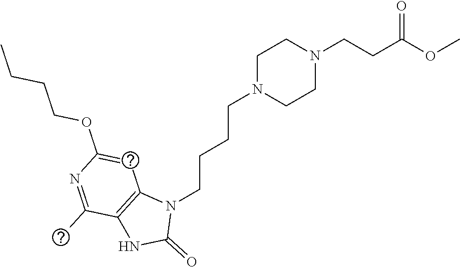 [SMILES: CCCCOc1cc2c([nH]c(=O)n2CCCCN2CCN(CCC(=O)OC)CC2)c(C)n1]

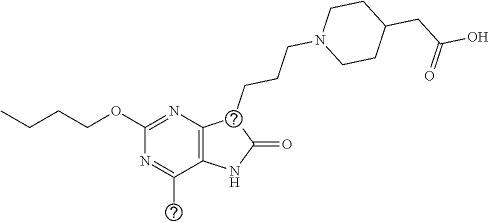 [SMILES: CCCCOc1nc(C)c2c(n1)C(CCCN1CCC(CC(=O)O)CC1)C(=O)N2]

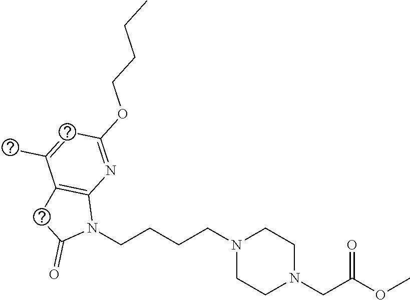

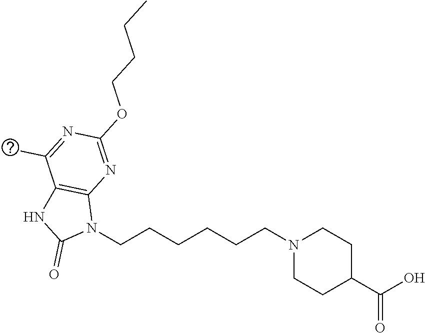 [SMILES: CCCCOc1nc(C)c2[nH]c(=O)n(CCCCCCN3CCC(C(=O)O)CC3)c2n1]

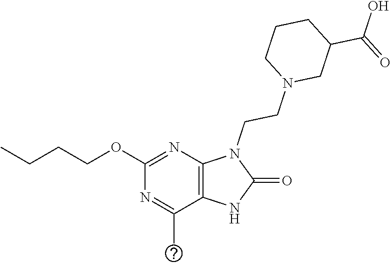 [SMILES: CCCCOc1nc(C)c2[nH]c(=O)n(CCN3CCCC(C(=O)O)C3)c2n1]

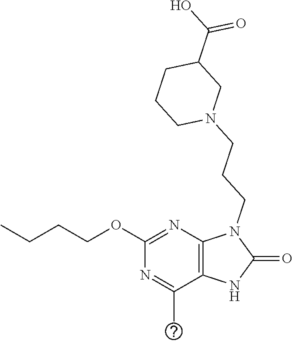 [SMILES: CCCCOc1nc(C)c2[nH]c(=O)n(CCCN3CCCC(C(=O)O)C3)c2n1]

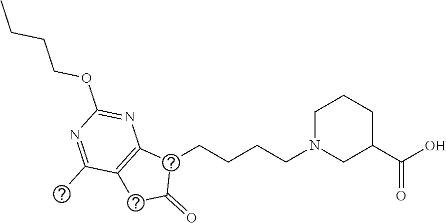 [SMILES: CCCCOc1nc(C)c2c(n1)C(CCCCN1CCCC(C(=O)O)C1)C(=O)C2]

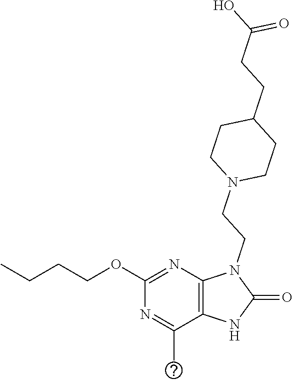 [SMILES: CCCCOc1nc(C)c2[nH]c(=O)n(CCN3CCC(CCC(=O)O)CC3)c2n1]

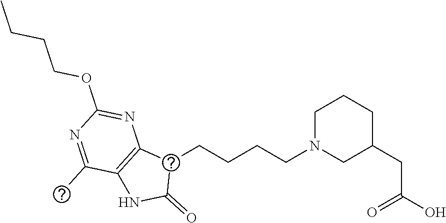 [SMILES: CCCCOc1nc(C)c2c(n1)C(CCCCN1CCCC(CC(=O)O)C1)C(=O)N2]

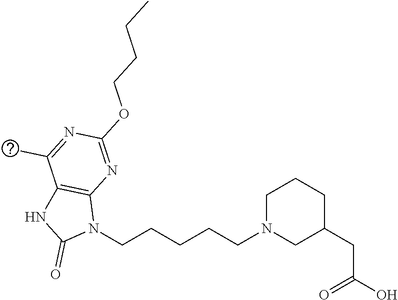 [SMILES: CCCCOc1nc(C)c2[nH]c(=O)n(CCCCCN3CCCC(CC(=O)O)C3)c2n1]

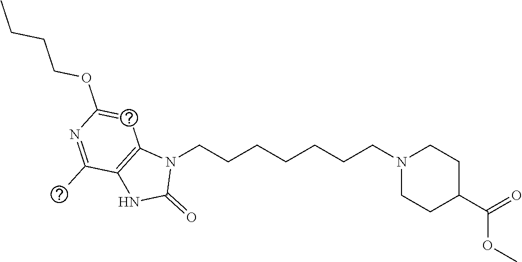 [SMILES: CCCCOc1cc2c([nH]c(=O)n2CCCCCCCN2CCC(C(=O)OC)CC2)c(C)n1]

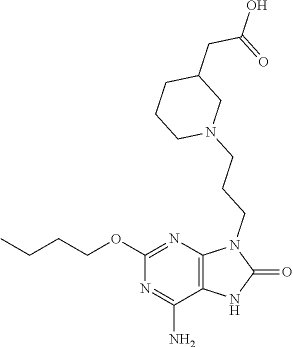 [SMILES: CCCCOc1nc(N)c2[nH]c(=O)n(CCCN3CCCC(CC(=O)O)C3)c2n1]

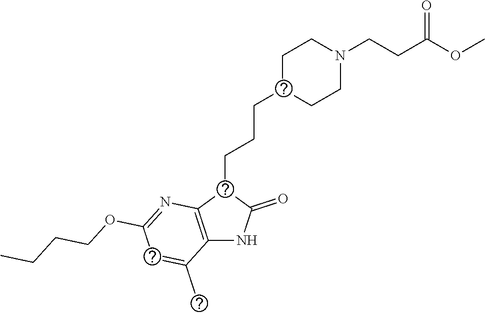 [SMILES: CCCCOc1cc(C)c2c(n1)C(CCCC1CCN(CCC(=O)OC)CC1)C(=O)N2]

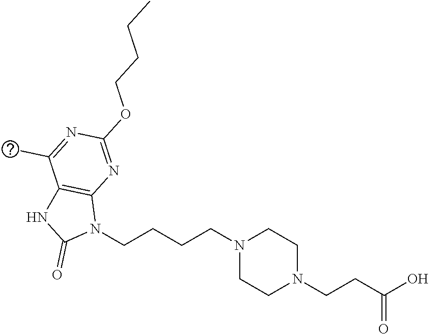 [SMILES: CCCCOc1nc(C)c2[nH]c(=O)n(CCCCN3CCN(CCC(=O)O)CC3)c2n1]

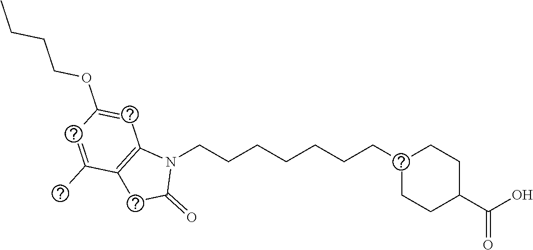 [SMILES: CCCCOc1cc(C)c2c(c1)N(CCCCCCCC1CCC(C(=O)O)CC1)C(=O)C2]

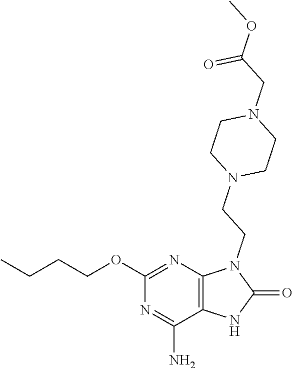 [SMILES: CCCCOc1nc(N)c2[nH]c(=O)n(CCN3CCN(CC(=O)OC)CC3)c2n1]

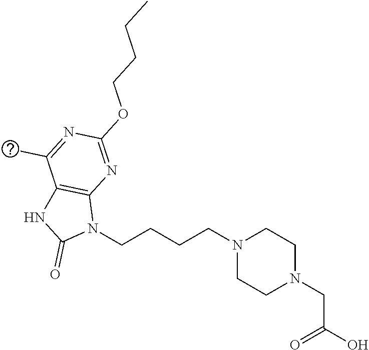 [SMILES: CCCCOc1nc(C)c2[nH]c(=O)n(CCCCN3CCN(CC(=O)O)CC3)c2n1]

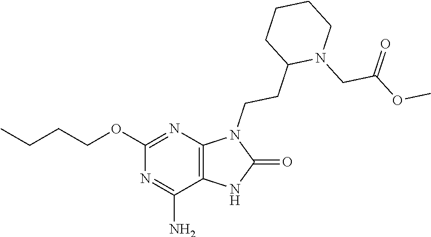 [SMILES: CCCCOc1nc(N)c2[nH]c(=O)n(CCC3CCCCN3CC(=O)OC)c2n1]

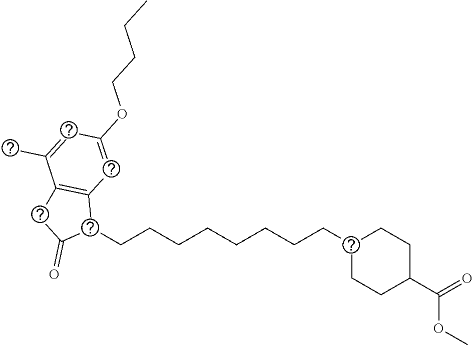

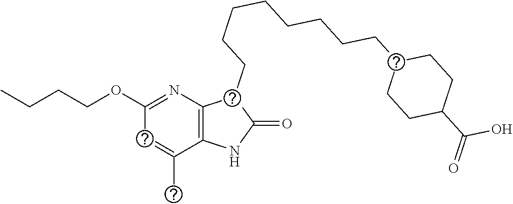 [SMILES: CCCCOc1cc(C)c2c(n1)C(CCCCCCCCC1CCC(C(=O)O)CC1)C(=O)N2]

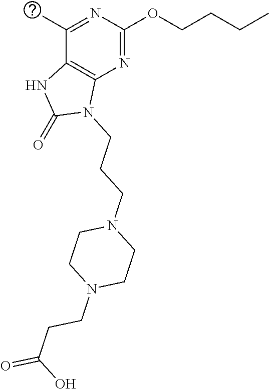 [SMILES: CCCCOc1nc(C)c2[nH]c(=O)n(CCCN3CCN(CCC(=O)O)CC3)c2n1]

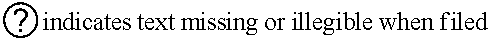

In some embodiments, the compound is Telratolimod, a TLR-7/TLR8 agonist which is chemically designated N-(4-((4-amino-2-butyl-1H-imidazo[4,5-c]quinolin-1-yl)oxy)butyl)stearamide. Telratolimod was previously studied in clinical trials for the treatment of cancer. Telratolimod is represented by the structure:

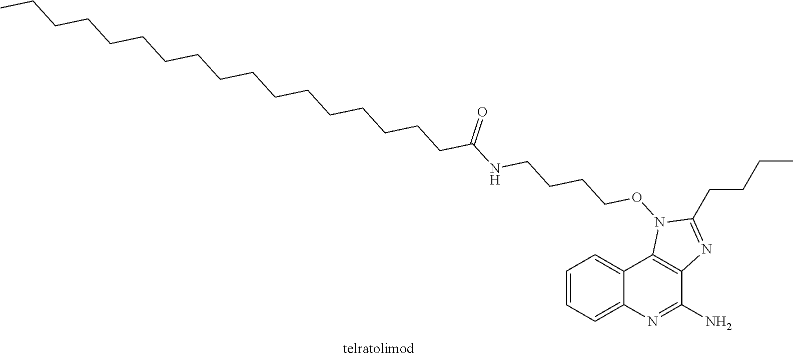 [SMILES: CCCCCCCCCCCCCCCCCC(=O)NCCCCOn1c(CCCC)nc2c(N)nc3ccccc3c21]

In some embodiments, telratolimod is administered intramuscularly (IM). In other embodiments, telratolimod is administered intramuscularly (IM) at a dose between 1 microgram and 10 micrograms. In other embodiments, telratolimod is administered intramuscularly (IM) at a dose of 1 microgram. In other embodiments, telratolimod is administered intramuscularly (IM) at a dose of 5 micrograms.

Telratolimod and/or structurally related compounds are described in PCT International Patent Applications WO2019/047824, WO2020/023680, and WO2015/162075, the contents of each of which are hereby incorporated by reference for all purposes and the specific purposes identified herein. In some embodiments, such compounds are represented by any one or more of the structures shown in Table 7. Any one of the compounds depicted in Table 7 is suitable for use in the methods of the present disclosure.

TABLE 7
Compound

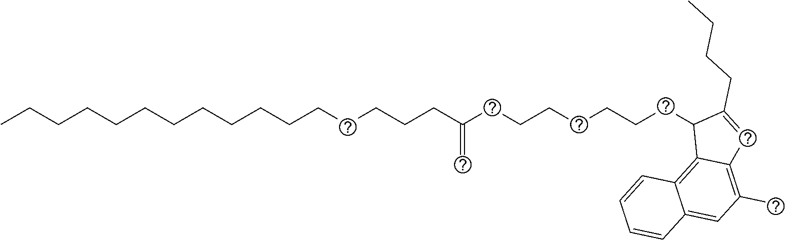 [SMILES: C=C(CCCCCCCCCCCCCCCC)CCCCCCCC1C(CCCC)=Cc2c(C)cc3ccccc3c21]

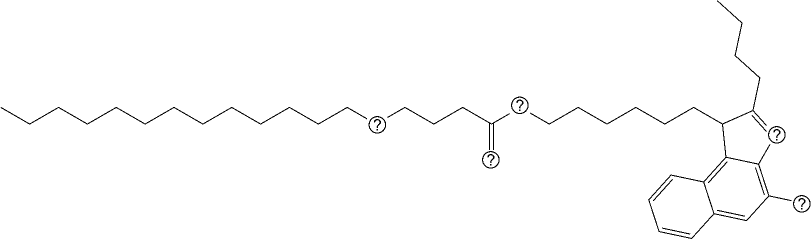 [SMILES: C=C(CCCCCCCCCCCCCCCCC)CCCCCCCC1C(CCCC)=Cc2c(C)cc3ccccc3c21]

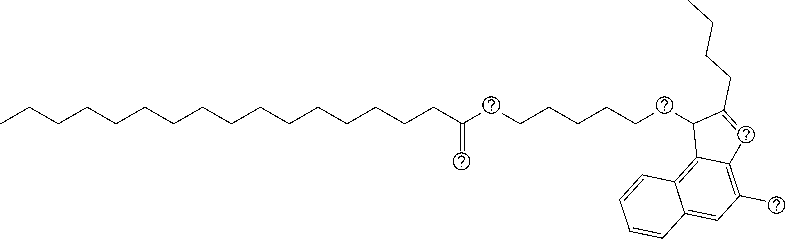 [SMILES: C=C(CCCCCCCCCCCCCCCC)CCCCCCCC1C(CCCC)=Cc2c(C)cc3ccccc3c21]

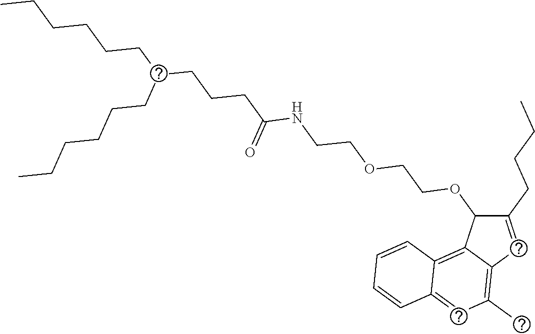 [SMILES: CCCCCCC(CCCCCC)CCCC(=O)NCCOCCOC1C(CCCC)=Cc2c(C)cc3ccccc3c21]

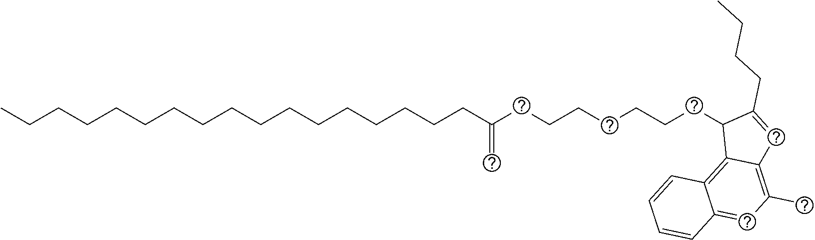 [SMILES: C=C(CCCCCCCCCCCCCCCCC)CCCCCCCC1C(CCCC)=Cc2c(C)cc3ccccc3c21]

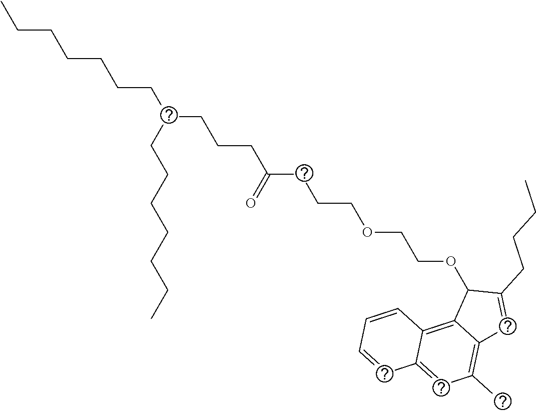 [SMILES: CCCCCCCC(CCCCCCC)CCCC(=O)CCCOCCOC1C(CCCC)=Cc2c(C)cc3ccccc3c21]

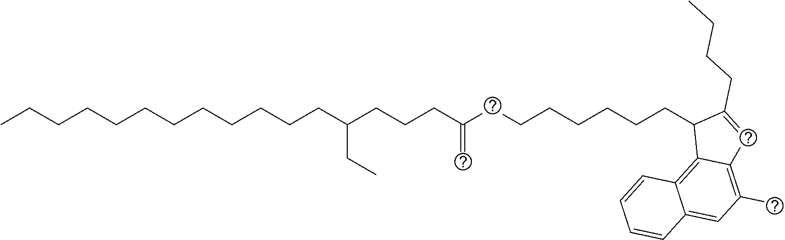 [SMILES: C=C(CCCCCCCC1C(CCCC)=Cc2c(C)cc3ccccc3c21)CCCC(CC)CCCCCCCCCCCC]

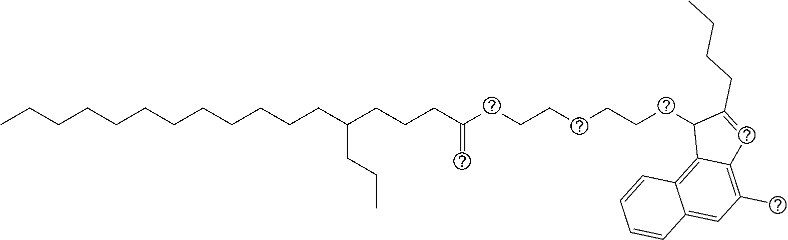 [SMILES: C=C(CCCCCCCC1C(CCCC)=Cc2c(C)cc3ccccc3c21)CCCC(CCC)CCCCCCCCCCCC]

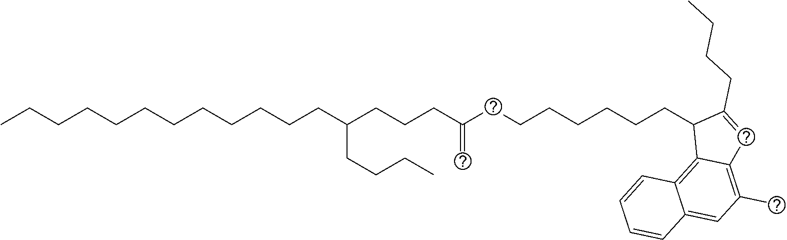 [SMILES: C=C(CCCCCCCC1C(CCCC)=Cc2c(C)cc3ccccc3c21)CCCC(CCCC)CCCCCCCCCCCC]

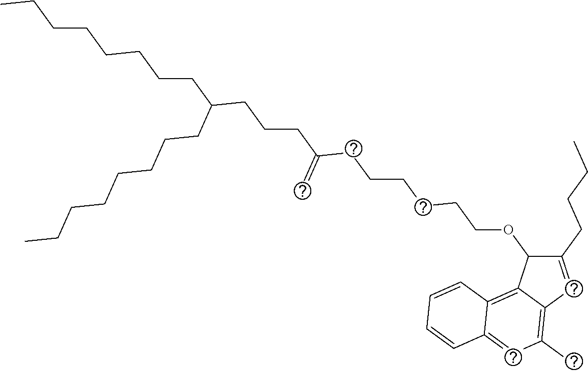 [SMILES: C=C(CCCCCCOC1C(CCCC)=Cc2c(C)cc3ccccc3c21)CCCC(CCCCCCCC)CCCCCCCC]

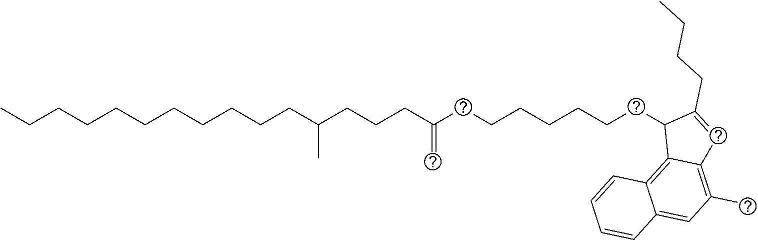 [SMILES: C=C(CCCCCCCC1C(CCCC)=Cc2c(C)cc3ccccc3c21)CCCC(C)CCCCCCCCCCC]

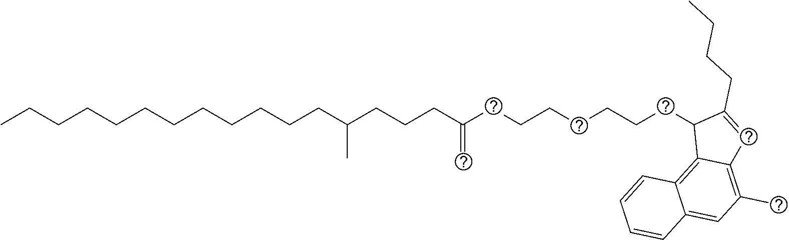 [SMILES: C=C(CCCCCCCC1C(CCCC)=Cc2c(C)cc3ccccc3c21)CCCC(C)CCCCCCCCCCCC]

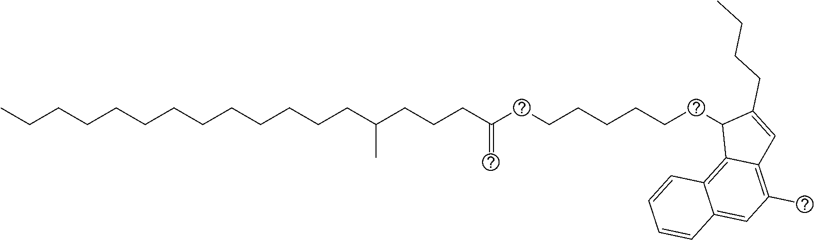 [SMILES: C=C(CCCCCCCC1C(CCCC)=Cc2c(C)cc3ccccc3c21)CCCC(C)CCCCCCCCCCCCC]

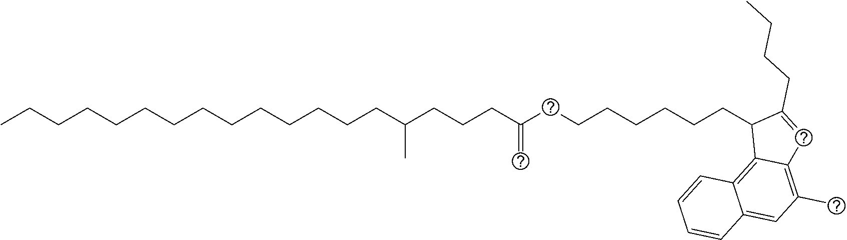 [SMILES: C=C(CCCCCCCC1C(CCCC)=Cc2c(C)cc3ccccc3c21)CCCC(C)CCCCCCCCCCCCCC]

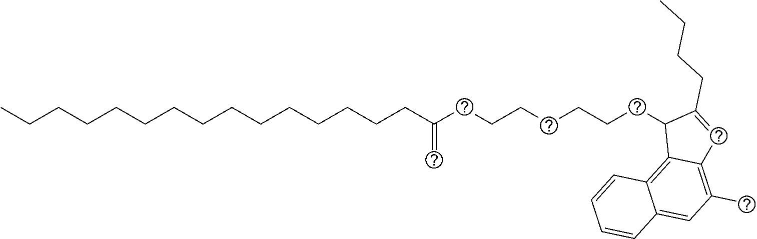 [SMILES: C=C(CCCCCCCCCCCCCCC)CCCCCCCC1C(CCCC)=Cc2c(C)cc3ccccc3c21]

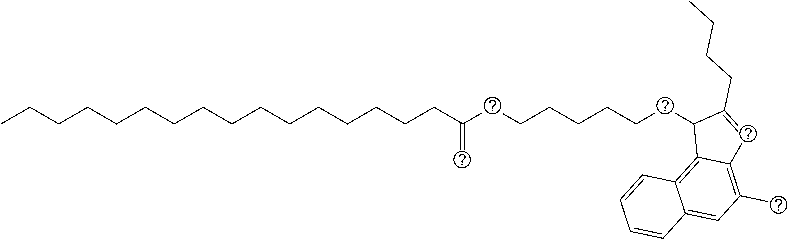 [SMILES: C=C(CCCCCCCCCCCCCCCC)CCCCCCCC1C(CCCC)=Cc2c(C)cc3ccccc3c21]

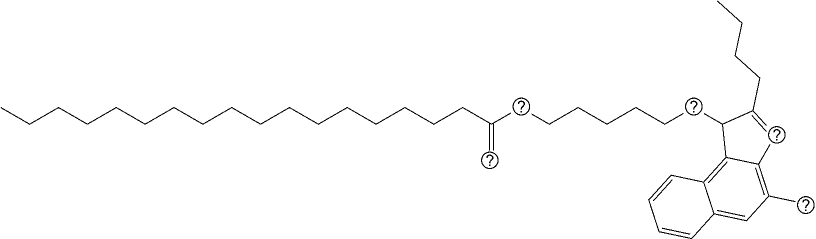 [SMILES: C=C(CCCCCCCCCCCCCCCCC)CCCCCCCC1C(CCCC)=Cc2c(C)cc3ccccc3c21]

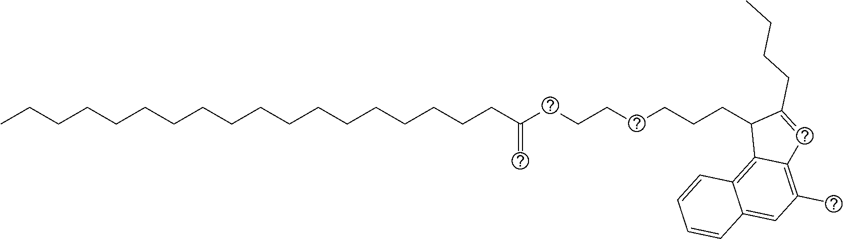 [SMILES: C=C(CCCCCCCCCCCCCCCCCC)CCCCCCCC1C(CCCC)=Cc2c(C)cc3ccccc3c21]

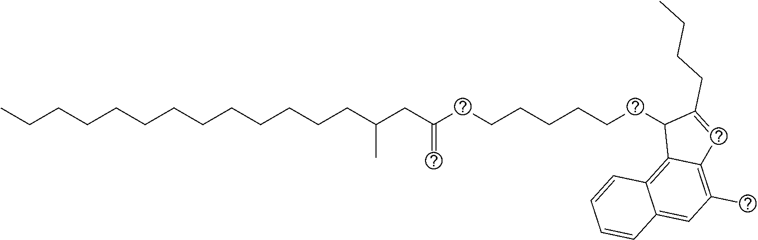 [SMILES: C=C(CCCCCCCC1C(CCCC)=Cc2c(C)cc3ccccc3c21)CC(C)CCCCCCCCCCCCC]

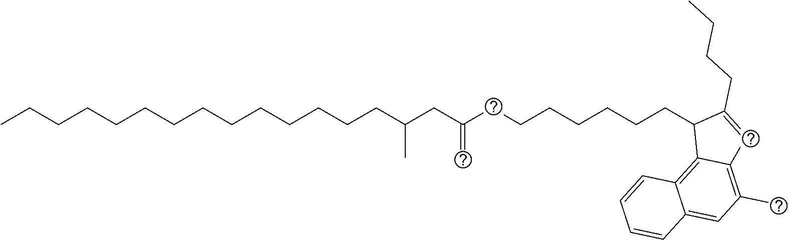 [SMILES: C=C(CCCCCCCC1C(CCCC)=Cc2c(C)cc3ccccc3c21)CC(C)CCCCCCCCCCCCCC]

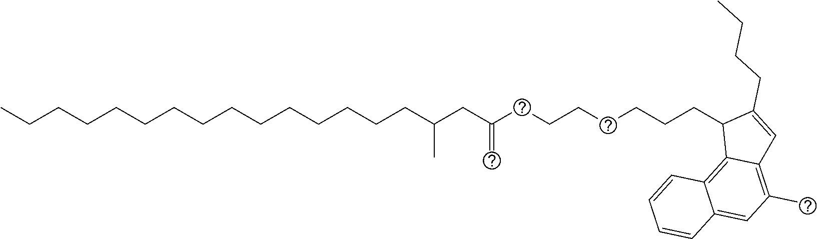 [SMILES: C=C(CCCCCCCC1C(CCCC)=Cc2c(C)cc3ccccc3c21)CC(C)CCCCCCCCCCCCCCC]

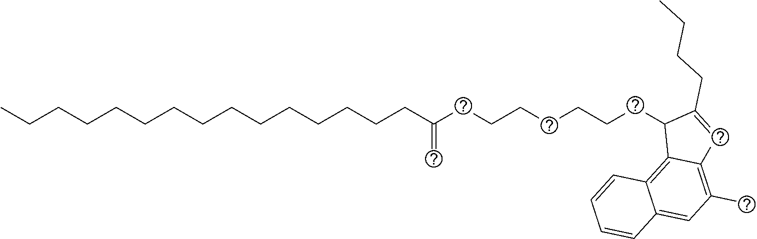 [SMILES: C=C(CCCCCCCCCCCCCCC)CCCCCCCC1C(CCCC)=Cc2c(C)cc3ccccc3c21]

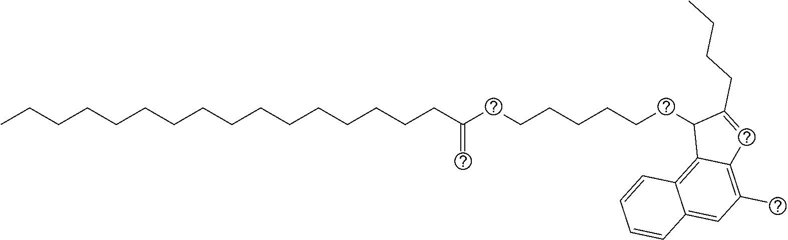 [SMILES: C=C(CCCCCCCCCCCCCCCC)CCCCCCCC1C(CCCC)=Cc2c(C)cc3ccccc3c21]

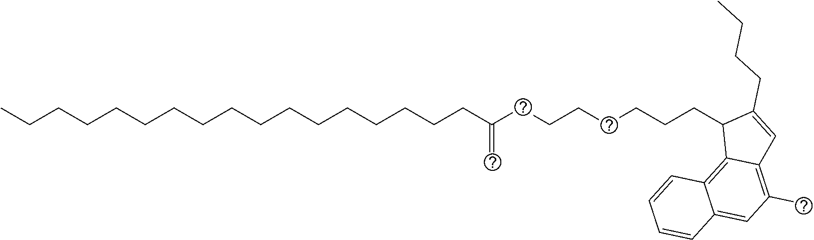 [SMILES: C=C(CCCCCCCCCCCCCCCCC)CCCCCCCC1C(CCCC)=Cc2c(C)cc3ccccc3c21]

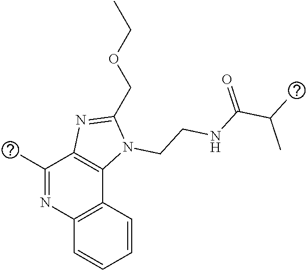 [SMILES: CCOCc1nc2c(C)nc3ccccc3c2n1CCNC(=O)C(C)C]

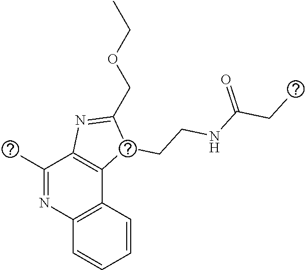 [SMILES: CCOCC1=Nc2c(C)nc3ccccc3c2C1CCNC(=O)CC]

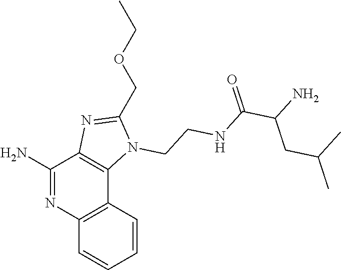 [SMILES: CCOCc1nc2c(N)nc3ccccc3c2n1CCNC(=O)C(N)CC(C)C]

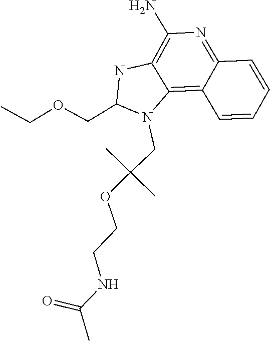 [SMILES: CCOCC1Nc2c(N)nc3ccccc3c2N1CC(C)(C)OCCNC(C)=O]

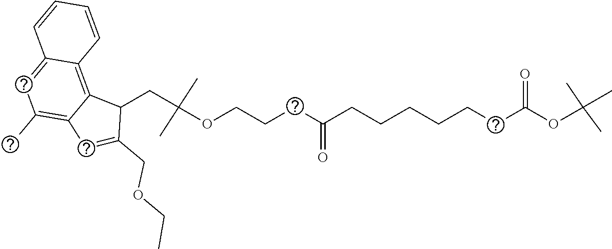 [SMILES: CCOCC1=Cc2c(C)cc3ccccc3c2C1CC(C)(C)OCCCC(=O)CCCCCCC(=O)OC(C)(C)C]

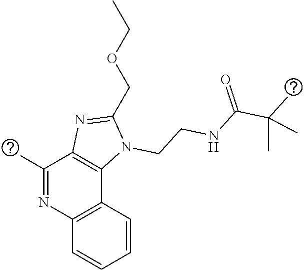 [SMILES: CCOCc1nc2c(C)nc3ccccc3c2n1CCNC(=O)C(C)(C)C]

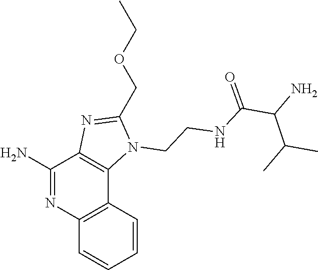 [SMILES: CCOCc1nc2c(N)nc3ccccc3c2n1CCNC(=O)C(N)C(C)C]

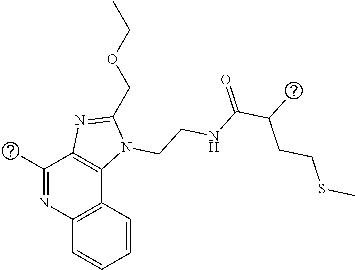

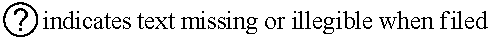

In some embodiments, the compound is Isatoribine, a TLR-7 agonist which is chemically designated N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione. Isatoribine was previously studied in clinical trials for the treatment of chronic hepatitis C virus (HCV). Isatoribine is represented by the structure:

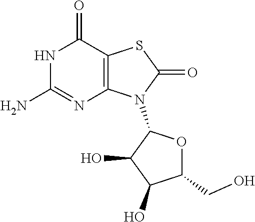 [SMILES: Nc1nc2c(sc(=O)n2[C@@H]2O[C@H](CO)[C@@H](O)[C@H]2O)c(=O)[nH]1]

In other embodiments, isatoribine is administered intravenously. In some embodiments, isatoribine is administered at a dose between 200 mg and 1,000 mg per dose. In some embodiments, isatoribine is administered once daily. In some embodiments, isatoribine is administered twice daily. In some embodiments, isatoribine is administered thrice daily.

Isatoribine and/or structurally related compounds are described in PCT International Patent Applications WO 2016/091698 and WO 2016/180743, the contents of each of which are hereby incorporated by reference for all purposes and the specific purposes identified herein. In some embodiments, such compounds are represented by any one or more of the structures shown in Table 8. Any one of the compounds depicted in Table 8 is suitable for use in the methods of the present disclosure.

TABLE 8
Compound

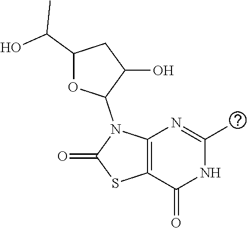 [SMILES: Cc1nc2c(sc(=O)n2C2OC(C(C)O)CC2O)c(=O)[nH]1]

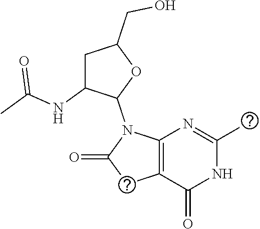 [SMILES: CC(=O)NC1CC(CO)OC1N1C(=O)Cc2c1nc(C)[nH]c2=O]

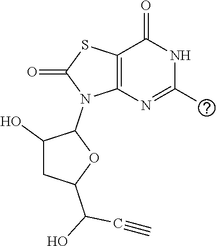 [SMILES: C#CC(O)C1CC(O)C(n2c(=O)sc3c(=O)[nH]c(C)nc32)O1]

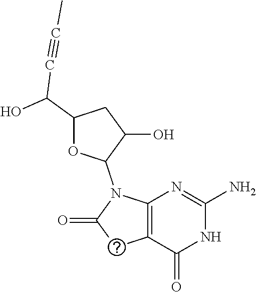 [SMILES: CC#CC(O)C1CC(O)C(N2C(=O)Cc3c2nc(N)[nH]c3=O)O1]

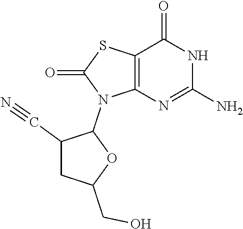 [SMILES: N#CC1CC(CO)OC1n1c(=O)sc2c(=O)[nH]c(N)nc21]

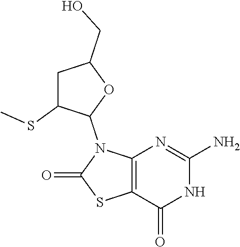 [SMILES: CSC1CC(CO)OC1n1c(=O)sc2c(=O)[nH]c(N)nc21]

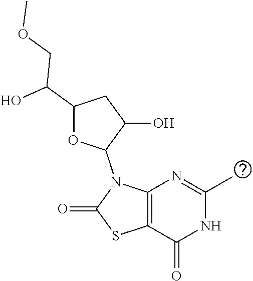 [SMILES: COCC(O)C1CC(O)C(n2c(=O)sc3c(=O)[nH]c(C)nc32)O1]

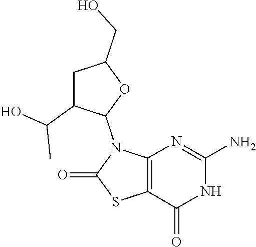 [SMILES: CC(O)C1CC(CO)OC1n1c(=O)sc2c(=O)[nH]c(N)nc21]

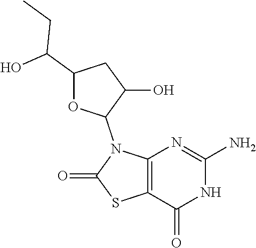 [SMILES: CCC(O)C1CC(O)C(n2c(=O)sc3c(=O)[nH]c(N)nc32)O1]

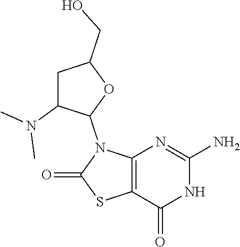 [SMILES: CN(C)C1CC(CO)OC1n1c(=O)sc2c(=O)[nH]c(N)nc21]

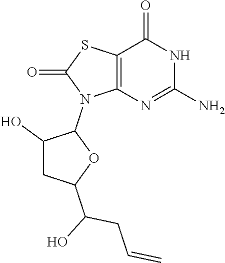 [SMILES: C=CCC(O)C1CC(O)C(n2c(=O)sc3c(=O)[nH]c(N)nc32)O1]

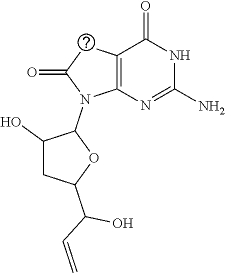 [SMILES: C=CC(O)C1CC(O)C(N2C(=O)Cc3c2nc(N)[nH]c3=O)O1]

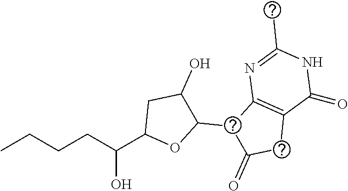 [SMILES: CCCCC(O)C1CC(O)C(C2C(=O)Cc3c2nc(C)[nH]c3=O)O1]

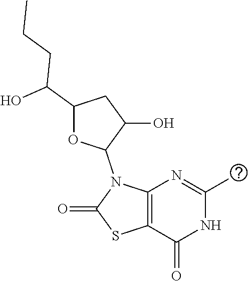 [SMILES: CCCC(O)C1CC(O)C(n2c(=O)sc3c(=O)[nH]c(C)nc32)O1]

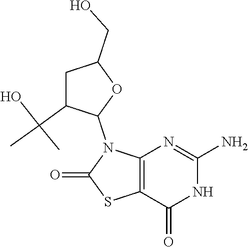 [SMILES: CC(C)(O)C1CC(CO)OC1n1c(=O)sc2c(=O)[nH]c(N)nc21]

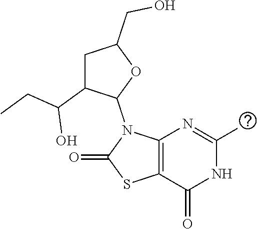 [SMILES: CCC(O)C1CC(CO)OC1n1c(=O)sc2c(=O)[nH]c(C)nc21]

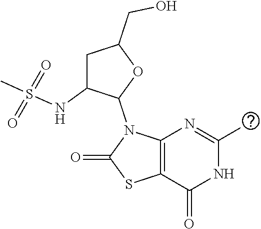

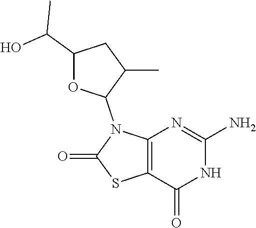 [SMILES: CC(O)C1CC(C)C(n2c(=O)sc3c(=O)[nH]c(N)nc32)O1]

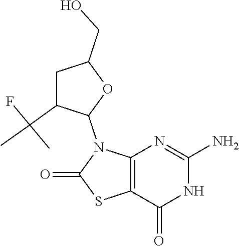 [SMILES: CC(C)(F)C1CC(CO)OC1n1c(=O)sc2c(=O)[nH]c(N)nc21]

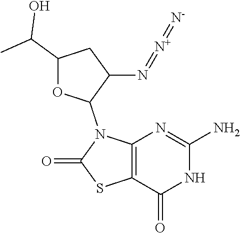 [SMILES: CC(O)C1CC(N=[N+]=[N-])C(n2c(=O)sc3c(=O)[nH]c(N)nc32)O1]

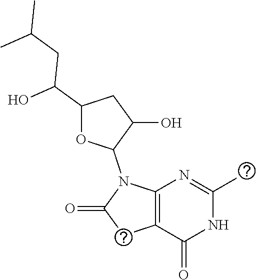 [SMILES: Cc1nc2c(c(=O)[nH]1)CC(=O)N2C1OC(C(O)CC(C)C)CC1O]

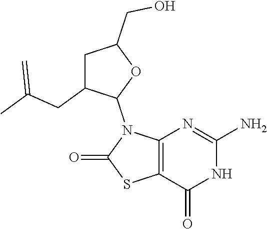 [SMILES: C=C(C)CC1CC(CO)OC1n1c(=O)sc2c(=O)[nH]c(N)nc21]

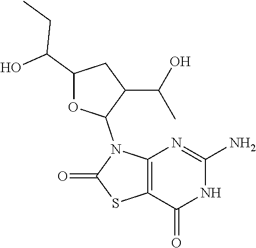 [SMILES: CCC(O)C1CC(C(C)O)C(n2c(=O)sc3c(=O)[nH]c(N)nc32)O1]

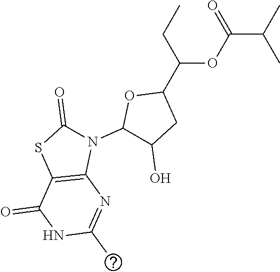 [SMILES: CCC(OC(=O)C(C)C)C1CC(O)C(n2c(=O)sc3c(=O)[nH]c(C)nc32)O1]

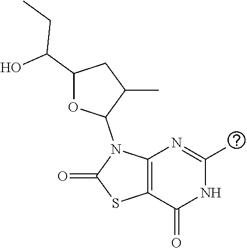 [SMILES: CCC(O)C1CC(C)C(n2c(=O)sc3c(=O)[nH]c(C)nc32)O1]

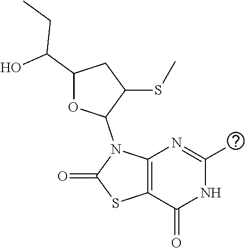 [SMILES: CCC(O)C1CC(SC)C(n2c(=O)sc3c(=O)[nH]c(C)nc32)O1]

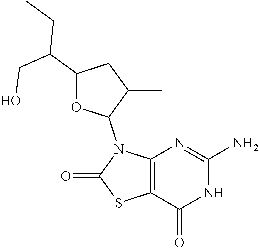 [SMILES: CCC(CO)C1CC(C)C(n2c(=O)sc3c(=O)[nH]c(N)nc32)O1]

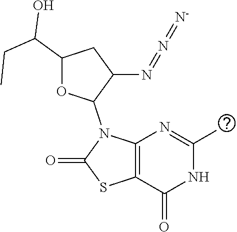

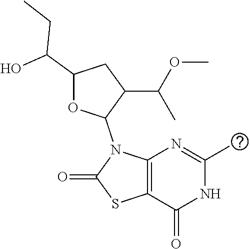 [SMILES: CCC(O)C1CC(C(C)OC)C(n2c(=O)sc3c(=O)[nH]c(C)nc32)O1]

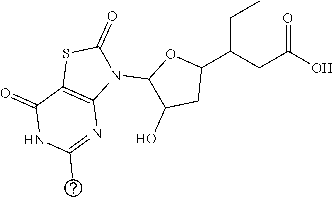 [SMILES: CCC(CC(=O)O)C1CC(O)C(n2c(=O)sc3c(=O)[nH]c(C)nc32)O1]

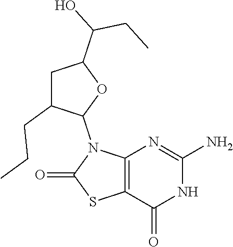 [SMILES: CCCC1CC(C(O)CC)OC1n1c(=O)sc2c(=O)[nH]c(N)nc21]

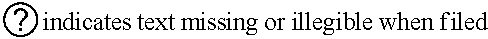

In some embodiments, the compound is N-[4-(4-amino-2-ethyl-1H-imidazo[4,5-c]quinolin-1-yl)butyl]-methanesulfonamide, a TLR-7 agonist also known as 3M-852A or PF-04878691. 3M-852A was previously studied in clinical trials for the treatment of various cancers. 3M-852A is represented by the structure:

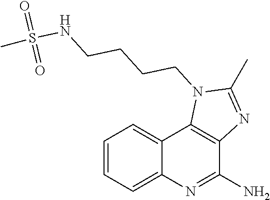 [SMILES: Cc1nc2c(N)nc3ccccc3c2n1CCCCNS(C)(=O)=O]

In some embodiments, 3M-852A is administered orally. In some embodiments, 3M-852A is administered intravenously. In some embodiments, 3M-852A is administered intravenously at dose ranges of about 0.15 to about 2.0 mg/m².

3M-852A and/or structurally related compounds are described in PCT International Patent Applications WO 2017/040233, WO 2017/040234, WO 2017/184735 and WO 2020/023680, the contents of each of which are hereby incorporated by reference for all purposes and the specific purposes identified herein. In some embodiments, such compounds are represented by any one or more of the structures shown in Table 9. Any one of the compounds depicted in Table 9 is suitable for use in the methods of the present disclosure.

TABLE 9
Compound

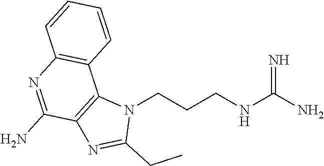 [SMILES: CCc1nc2c(N)nc3ccccc3c2n1CCCNC(=N)N]

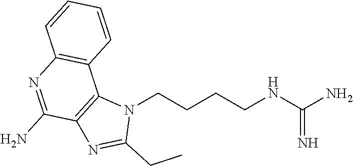 [SMILES: CCc1nc2c(N)nc3ccccc3c2n1CCCCNC(=N)N]

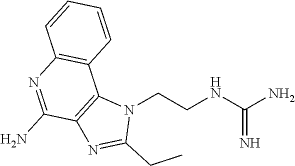 [SMILES: CCc1nc2c(N)nc3ccccc3c2n1CCNC(=N)N]

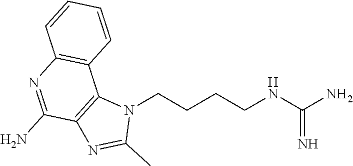 [SMILES: Cc1nc2c(N)nc3ccccc3c2n1CCCCNC(=N)N]

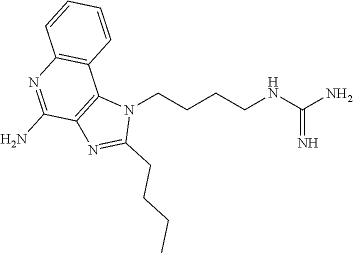 [SMILES: CCCCc1nc2c(N)nc3ccccc3c2n1CCCCNC(=N)N]

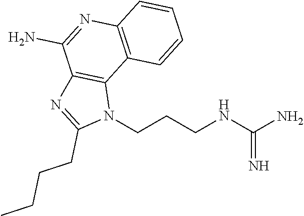 [SMILES: CCCCc1nc2c(N)nc3ccccc3c2n1CCCNC(=N)N]

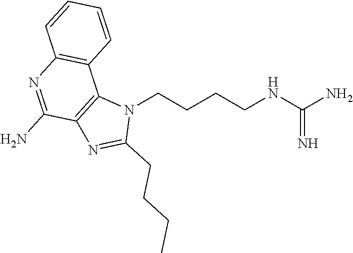 [SMILES: CCCCc1nc2c(N)nc3ccccc3c2n1CCCCNC(=N)N]

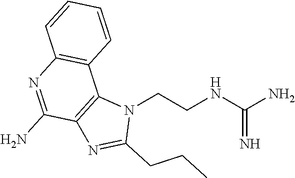 [SMILES: CCCc1nc2c(N)nc3ccccc3c2n1CCNC(=N)N]

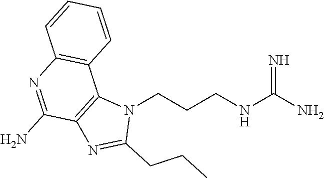 [SMILES: CCCc1nc2c(N)nc3ccccc3c2n1CCCNC(=N)N]

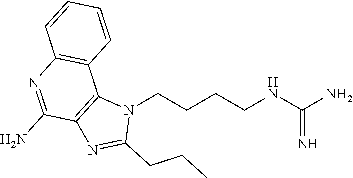 [SMILES: CCCc1nc2c(N)nc3ccccc3c2n1CCCCNC(=N)N]

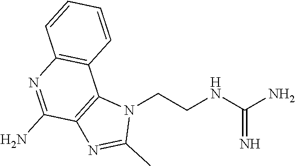 [SMILES: Cc1nc2c(N)nc3ccccc3c2n1CCNC(=N)N]

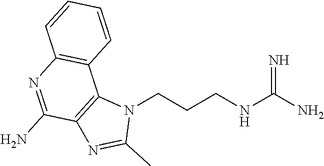 [SMILES: Cc1nc2c(N)nc3ccccc3c2n1CCCNC(=N)N]

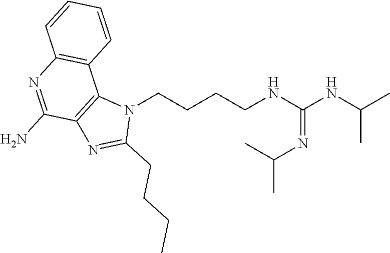 [SMILES: CCCCc1nc2c(N)nc3ccccc3c2n1CCCCN/C(=N\C(C)C)NC(C)C]

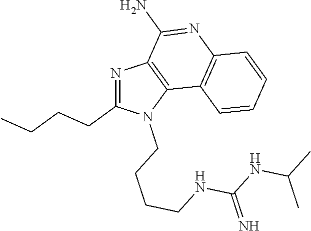 [SMILES: CCCCc1nc2c(N)nc3ccccc3c2n1CCCCNC(=N)NC(C)C]

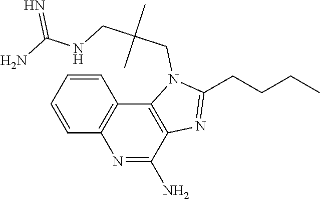 [SMILES: CCCCc1nc2c(N)nc3ccccc3c2n1CC(C)(C)CNC(=N)N]

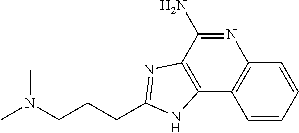 [SMILES: CN(C)CCCc1nc2c(N)nc3ccccc3c2[nH]1]

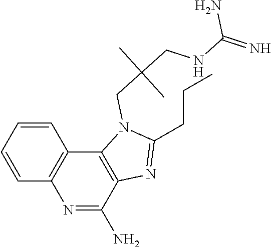 [SMILES: CCCc1nc2c(N)nc3ccccc3c2n1CC(C)(C)CNC(=N)N]

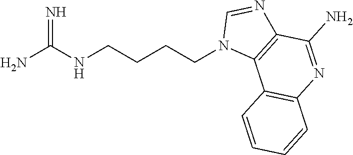 [SMILES: N=C(N)NCCCCn1cnc2c(N)nc3ccccc3c21]

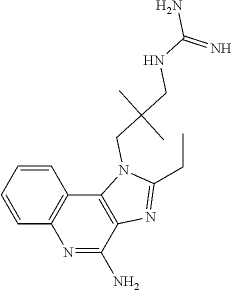 [SMILES: CCc1nc2c(N)nc3ccccc3c2n1CC(C)(C)CNC(=N)N]

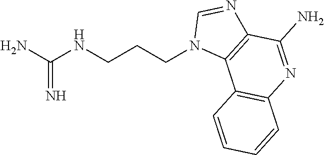 [SMILES: N=C(N)NCCCn1cnc2c(N)nc3ccccc3c21]

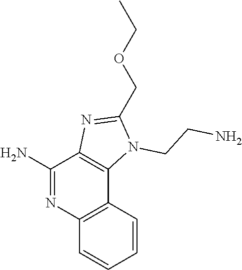 [SMILES: CCOCc1nc2c(N)nc3ccccc3c2n1CCN]

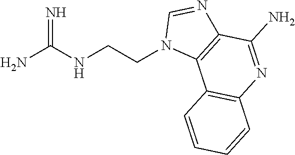 [SMILES: N=C(N)NCCn1cnc2c(N)nc3ccccc3c21]

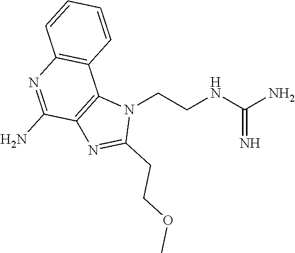 [SMILES: COCCc1nc2c(N)nc3ccccc3c2n1CCNC(=N)N]

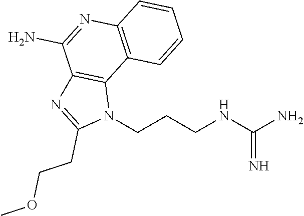 [SMILES: COCCc1nc2c(N)nc3ccccc3c2n1CCCNC(=N)N]

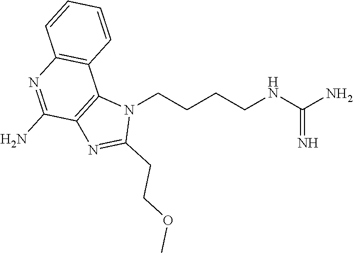 [SMILES: COCCc1nc2c(N)nc3ccccc3c2n1CCCCNC(=N)N]

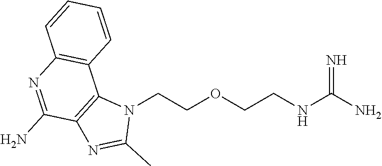 [SMILES: Cc1nc2c(N)nc3ccccc3c2n1CCOCCNC(=N)N]

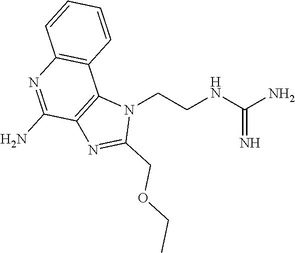 [SMILES: CCOCc1nc2c(N)nc3ccccc3c2n1CCNC(=N)N]

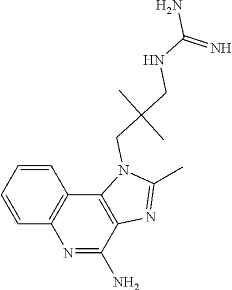 [SMILES: Cc1nc2c(N)nc3ccccc3c2n1CC(C)(C)CNC(=N)N]

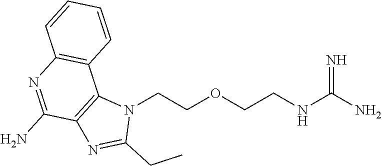 [SMILES: CCc1nc2c(N)nc3ccccc3c2n1CCOCCNC(=N)N]

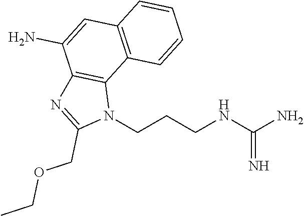 [SMILES: CCOCc1nc2c(N)cc3ccccc3c2n1CCCNC(=N)N]

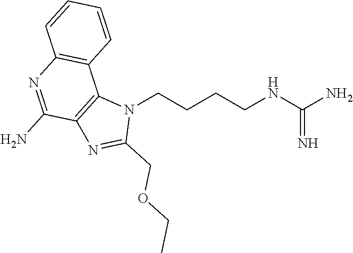 [SMILES: CCOCc1nc2c(N)nc3ccccc3c2n1CCCCNC(=N)N]

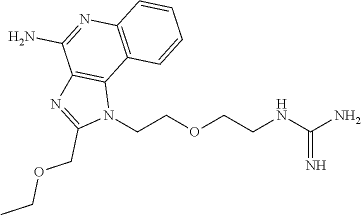 [SMILES: CCOCc1nc2c(N)nc3ccccc3c2n1CCOCCNC(=N)N]

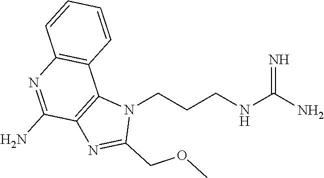 [SMILES: COCc1nc2c(N)nc3ccccc3c2n1CCCNC(=N)N]

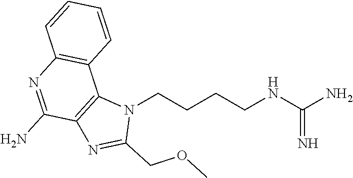 [SMILES: COCc1nc2c(N)nc3ccccc3c2n1CCCCNC(=N)N]

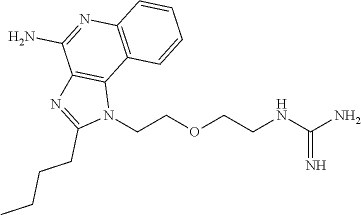 [SMILES: CCCCc1nc2c(N)nc3ccccc3c2n1CCOCCNC(=N)N]

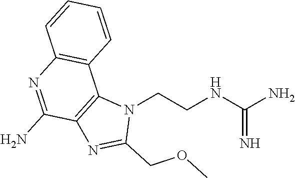 [SMILES: COCc1nc2c(N)nc3ccccc3c2n1CCNC(=N)N]

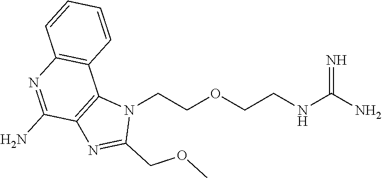 [SMILES: COCc1nc2c(N)nc3ccccc3c2n1CCOCCNC(=N)N]

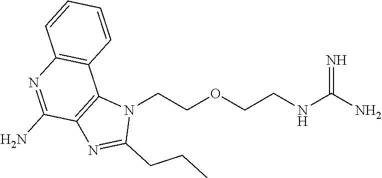 [SMILES: CCCc1nc2c(N)nc3ccccc3c2n1CCOCCNC(=N)N]

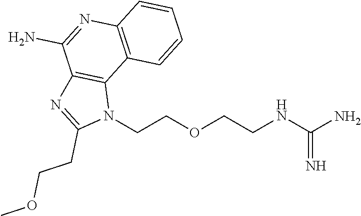 [SMILES: COCCc1nc2c(N)nc3ccccc3c2n1CCOCCNC(=N)N]

In some embodiments, the compound is Imiquimod, which is chemically designated 1-isobutyl-1H-imidazo[4,5-c]quinolin-4-amine. Imiquimod is marketed as a topical cream under the trade names Aldara® and Zyclara®. Aldara® is indicated for actinic keratoses (AK) in immunocompetent adults, basal cell carcinoma (sBCC) in immunocompetent adults, and external genital and perianal warts/condyloma acuminata in patients 12 years old or older. Zyclara® is indicated for the topical treatment of actinic keratoses (AK) in immunocompetent adults, and external genital and perianal warts/condyloma acuminata (EGW) in patients 12 years or older. Imiquimod is represented by the structure:

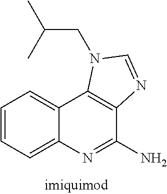 [SMILES: CC(C)Cn1cnc2c(N)nc3ccccc3c21]

In some embodiments, imiquimod is administered topically. In other embodiments, imiquimod is administered as a topical cream. In other embodiments, imiquimod is administered as a topical cream comprising about 0.5% by weight imiquimod. In other embodiments, imiquimod is administered as a topical cream comprising about 2.5% by weight imiquimod. In other embodiments, imiquimod is administered as a topical cream comprising about 3.75% by weight imiquimod. In other embodiments, imiquimod is administered as a topical cream comprising at least one excipient selected from the group consisting of isostearic acid, cetyl alcohol, stearyl alcohol, white petrolatum, polysorbate 60, sorbitan monostearate, glycerin, xanthan gum, water, benzyl alcohol, methylparaben, and propylparaben.

Imiquimod and/or structurally related compounds are described in PCT International Patent Applications WO 2017/040233, WO 2017/184735, WO 2017/040234 and WO 2020/023680, the contents of each of which are hereby incorporated by reference for all purposes and the specific purposes identified herein. In some embodiments, such compounds are represented by any one or more of the structures shown in Table 10. Any one of the compounds depicted in Table 10 is suitable for use in the methods of the present disclosure.

TABLE 10
Compound

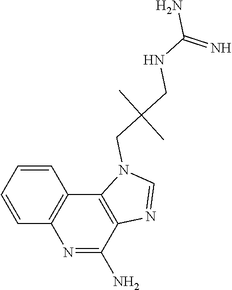 [SMILES: CC(C)(CNC(=N)N)Cn1cnc2c(N)nc3ccccc3c21]

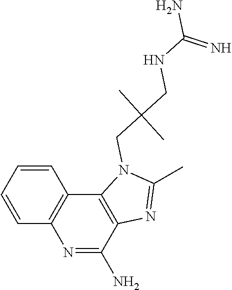 [SMILES: Cc1nc2c(N)nc3ccccc3c2n1CC(C)(C)CNC(=N)N]

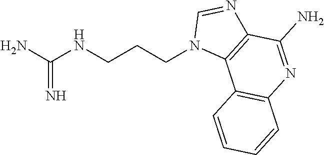 [SMILES: N=C(N)NCCCn1cnc2c(N)nc3ccccc3c21]

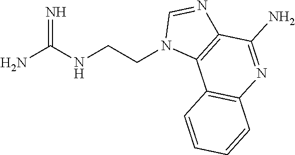 [SMILES: N=C(N)NCCn1cnc2c(N)nc3ccccc3c21]

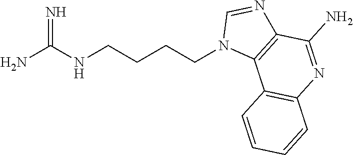 [SMILES: N=C(N)NCCCCn1cnc2c(N)nc3ccccc3c21]

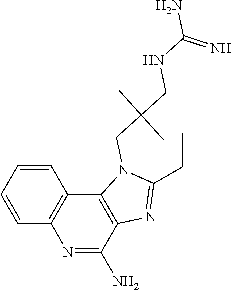 [SMILES: CCc1nc2c(N)nc3ccccc3c2n1CC(C)(C)CNC(=N)N]

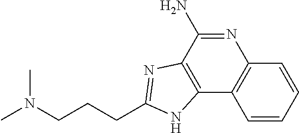 [SMILES: CN(C)CCCc1nc2c(N)nc3ccccc3c2[nH]1]

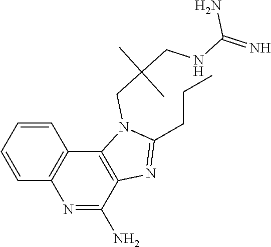 [SMILES: CCCc1nc2c(N)nc3ccccc3c2n1CC(C)(C)CNC(=N)N]

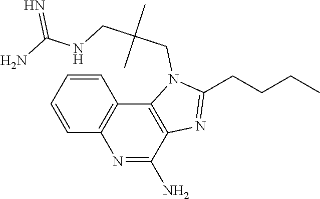 [SMILES: CCCCc1nc2c(N)nc3ccccc3c2n1CC(C)(C)CNC(=N)N]

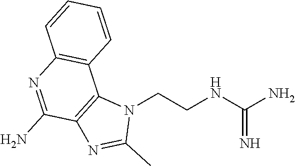 [SMILES: Cc1nc2c(N)nc3ccccc3c2n1CCNC(=N)N]

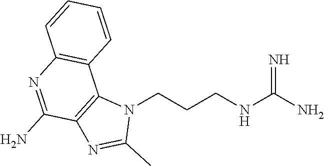 [SMILES: Cc1nc2c(N)nc3ccccc3c2n1CCCNC(=N)N]

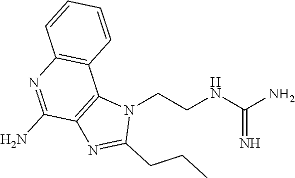 [SMILES: CCCc1nc2c(N)nc3ccccc3c2n1CCNC(=N)N]

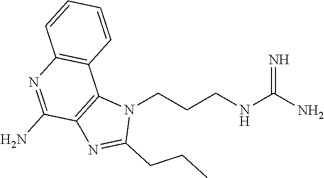 [SMILES: CCCc1nc2c(N)nc3ccccc3c2n1CCCNC(=N)N]

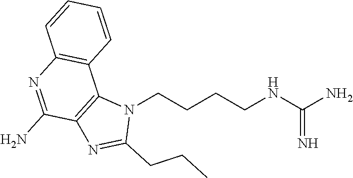 [SMILES: CCCc1nc2c(N)nc3ccccc3c2n1CCCCNC(=N)N]

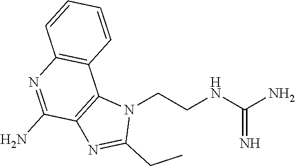 [SMILES: CCc1nc2c(N)nc3ccccc3c2n1CCNC(=N)N]

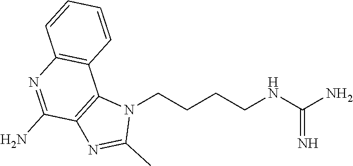 [SMILES: Cc1nc2c(N)nc3ccccc3c2n1CCCCNC(=N)N]

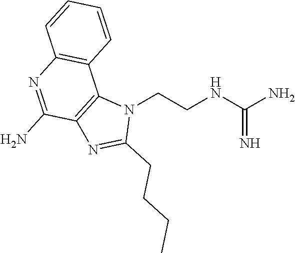 [SMILES: CCCCc1nc2c(N)nc3ccccc3c2n1CCNC(=N)N]

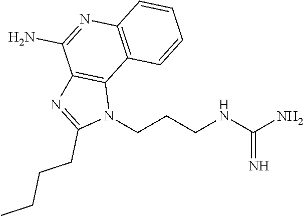 [SMILES: CCCCc1nc2c(N)nc3ccccc3c2n1CCCNC(=N)N]

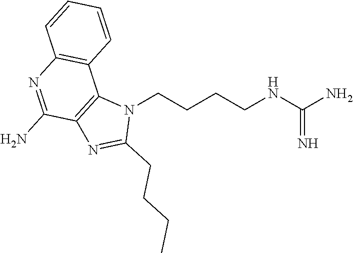 [SMILES: CCCCc1nc2c(N)nc3ccccc3c2n1CCCCNC(=N)N]

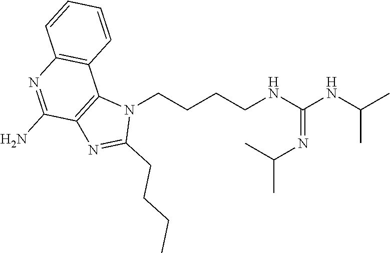 [SMILES: CCCCc1nc2c(N)nc3ccccc3c2n1CCCCN/C(=N\C(C)C)NC(C)C]

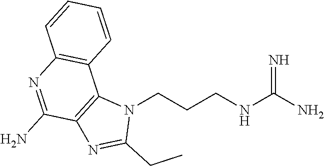 [SMILES: CCc1nc2c(N)nc3ccccc3c2n1CCCNC(=N)N]

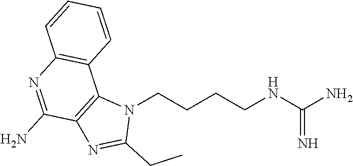 [SMILES: CCc1nc2c(N)nc3ccccc3c2n1CCCCNC(=N)N]

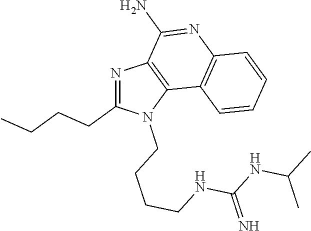 [SMILES: CCCCc1nc2c(N)nc3ccccc3c2n1CCCCNC(=N)NC(C)C]

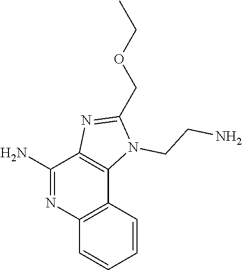 [SMILES: CCOCc1nc2c(N)nc3ccccc3c2n1CCN]

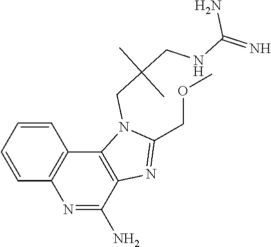 [SMILES: COCc1nc2c(N)nc3ccccc3c2n1CC(C)(C)CNC(=N)N]

In some embodiments, the compound is TMX-202, a TLR-7 agonist which is chemically designated 2-(4-((6-amino-2-(2-methoxyethoxy)-8-oxo-7H-purin-9(8H)methyl)benzamido)-ethyl-2,3-bis(oleoyloxy)propyl phosphate. TMX-202 was studied as a potential therapeutic for skin and bladder cancers. TMX-202 is represented by the structure:

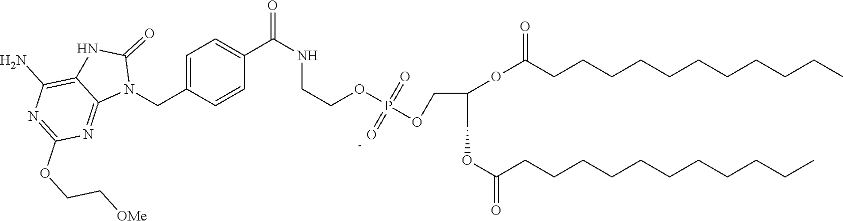

In some embodiments, TMX-202 is administered topically.

TMX-202 and/or structurally related compounds are described in PCT International Patent Applications WO 2011/134669 and WO 2015/023858, the contents of each of which are hereby incorporated by reference for all purposes and the specific purposes identified herein. In some embodiments, such compounds are represented by any one or more of the structures shown in Table 11. Any one of the compounds depicted in Table 11 is suitable for use in the methods of the present disclosure.

TABLE 11
Compound

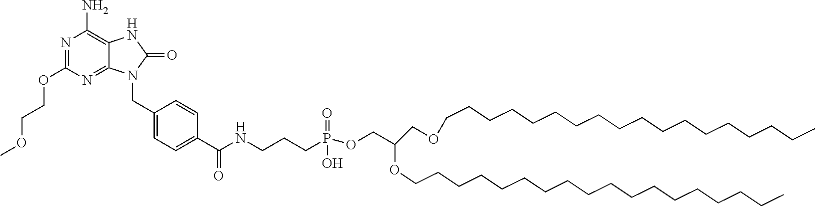 [SMILES: CCCCCCCCCCCCCCCCCCOCC(COP(=O)(O)CCCNC(=O)c1ccc(Cn2c(=O)[nH]c3c(N)nc(OCCOC)nc32)cc1)OCCCCCCCCCCCCCCCCCC]

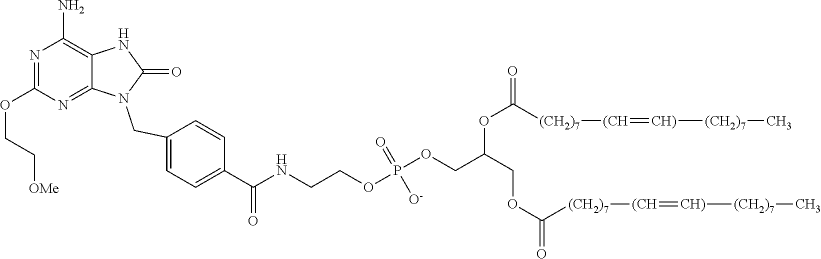

In some embodiments, the compound is methyl 2-(3-(((3-(6-amino-2-butoxy-8-oxo-7,8-dihydro-9H-purin-9-yl)propyl)(3-morpholinopropyl)amino)methyl)phenyl)acetate, a TLR-7 agonist also known as AZD-8848. AZD-8848 is in clinical trials as an inhaled nebulizer for the treatment of asthma and as a nasal formulation for the treatment of allergic rhinitis. AZD-8848 is represented by the structure:

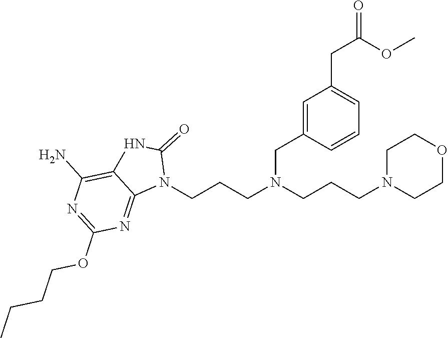 [SMILES: CCCCOc1nc(N)c2[nH]c(=O)n(CCCN(CCCN3CCOCC3)Cc3cccc(CC(=O)OC)c3)c2n1]

In some embodiments, AZD-8848 is administered as a nasal spray solution. In another embodiments, AZD08848 is administered as an inhaled nebuliser solution.

AZD-8848 and/or structurally related compounds are described in PCT International Patent Applications WO 2007/034882 and WO 2012/080730, the contents of each of which are hereby incorporated by reference for all purposes and the specific purposes identified herein. In some embodiments, such compounds are represented by any one or more of the structures shown in Table 12. Any one of the compounds depicted in Table 12 is suitable for use in the methods of the present disclosure.

TABLE 12
Compound

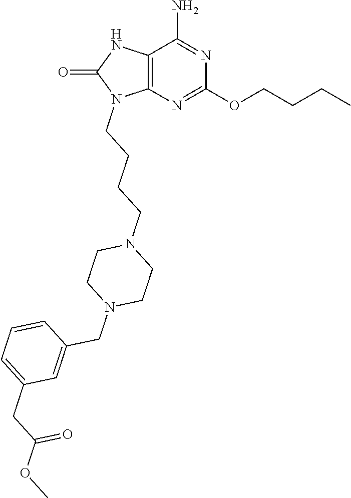 [SMILES: CCCCOc1nc(N)c2[nH]c(=O)n(CCCCN3CCN(Cc4cccc(CC(=O)OC)c4)CC3)c2n1]

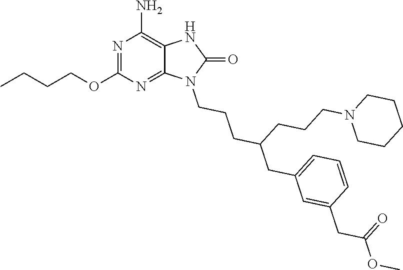 [SMILES: CCCCOc1nc(N)c2[nH]c(=O)n(CCCC(CCCN3CCCCC3)Cc3cccc(CC(=O)OC)c3)c2n1]

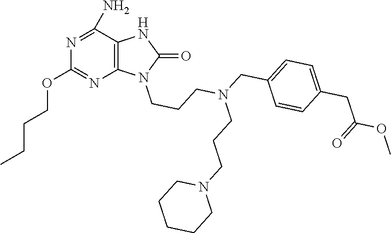 [SMILES: CCCCOc1nc(N)c2[nH]c(=O)n(CCCN(CCCN3CCCCC3)Cc3ccc(CC(=O)OC)cc3)c2n1]

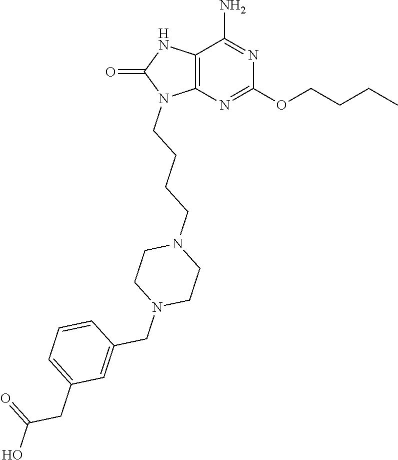 [SMILES: CCCCOc1nc(N)c2[nH]c(=O)n(CCCCN3CCN(Cc4cccc(CC(=O)O)c4)CC3)c2n1]

In some embodiments, the compound is 4-amino-1-benzyl-6-trifluoromethyl-1,3-dihydro-imidazo[4,5-c]pyridine-2-one, a TLR-7 agonist also known as PF-4171455. PF-4171455 was studied preclinically for the treatment of hepatitis-C virus (HCV) infection. PF-4171455 is represented by the structure:

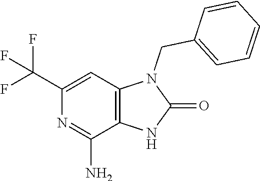 [SMILES: Nc1nc(C(F)(F)F)cc2c1[nH]c(=O)n2Cc1ccccc1]

PF-4171455 and/or structurally related compounds are described in PCT International Patent Application WO 2007/093901, the contents of which are hereby incorporated by reference for all purposes and the specific purposes identified herein. In some embodiments, such compounds are represented by any one or more of the structures shown in Table 13. Any one of the compounds depicted in Table 13 is suitable for use in the methods of the present disclosure.

TABLE 13
Compound

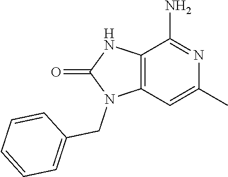 [SMILES: Cc1cc2c([nH]c(=O)n2Cc2ccccc2)c(N)n1]

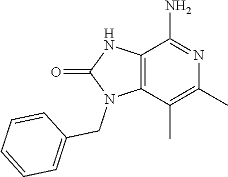 [SMILES: Cc1nc(N)c2[nH]c(=O)n(Cc3ccccc3)c2c1C]

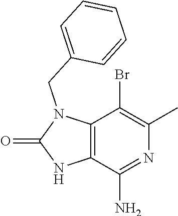 [SMILES: Cc1nc(N)c2[nH]c(=O)n(Cc3ccccc3)c2c1Br]

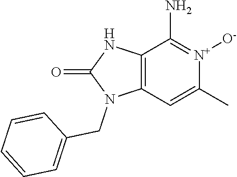 [SMILES: Cc1cc2c([nH]c(=O)n2Cc2ccccc2)c(N)[n+]1[O-]]

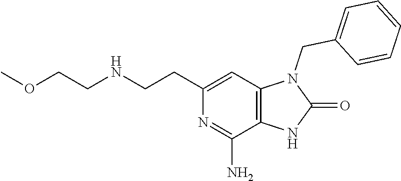 [SMILES: COCCNCCc1cc2c([nH]c(=O)n2Cc2ccccc2)c(N)n1]

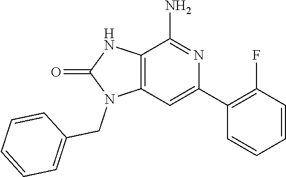 [SMILES: Nc1nc(-c2ccccc2F)cc2c1[nH]c(=O)n2Cc1ccccc1]

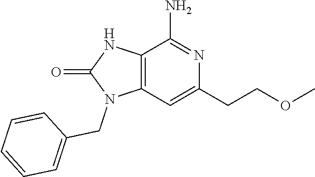 [SMILES: COCCc1cc2c([nH]c(=O)n2Cc2ccccc2)c(N)n1]

In some embodiments, the TLR-agonist is Epetirimod, a TLR-7 agonist which is chemically designated 1-isobutyl-1H-imidazo[4,5-c][1,5]naphthyridin-4-amine. Epetirimod (also known as S-30563 or TAK-851) was developed as a topical treatment for cervical dysplasia and human papillomavirus (HPV) infections. Epetirimod is represented by the structure:

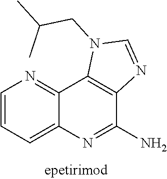 [SMILES: CC(C)Cn1cnc2c(N)nc3cccnc3c21]

In other embodiments, the TLR-agonist is Sotirimod, a TLR-7 agonist which is chemically designated 1-isobutyl-2-methyl-1H-imidazo[4,5-c][1,5]naphthyridin-4-amine. Sotirimod (also known as IRM 6 or S-30594) was developed as a topical treatment of actinic keratosis (AK). Sotirimod is represented by the structure:

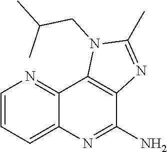 [SMILES: Cc1nc2c(N)nc3cccnc3c2n1CC(C)C]

Epetirimod, Sotirimod and/or structurally related compounds are described in PCT International Patent Application WO2017/040233, the contents of which are hereby incorporated by reference for all purposes and the specific purposes identified herein. In some embodiments, such compounds are represented by any one or more of the structures shown in Table 14. Any one of the compounds depicted in Table 14 is suitable for use in the methods of the present disclosure.

TABLE 14
Compound

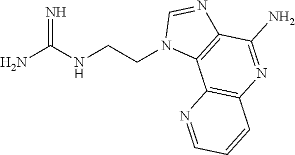 [SMILES: N=C(N)NCCn1cnc2c(N)nc3cccnc3c21]

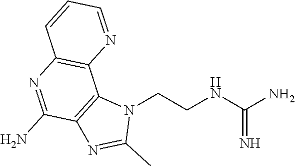 [SMILES: Cc1nc2c(N)nc3cccnc3c2n1CCNC(=N)N]

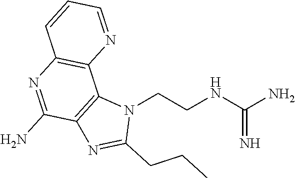 [SMILES: CCCc1nc2c(N)nc3cccnc3c2n1CCNC(=N)N]

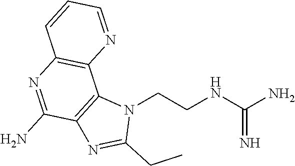 [SMILES: CCc1nc2c(N)nc3cccnc3c2n1CCNC(=N)N]

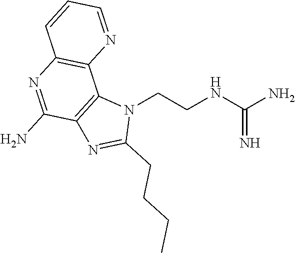 [SMILES: CCCCc1nc2c(N)nc3cccnc3c2n1CCNC(=N)N]

In some embodiments, the TLR-agonist is BIIB-021, which is chemically designated 1-isobutyl-2-methyl-1H-imidazo[4,5-c][1,5]naphthyridin-4-amine. BIIB-021 is a heat shock protein (Hsp) 90 inhibitor, which was studied in clinical trials for the treatment of various cancers. BIIB-021 is represented by the structure:

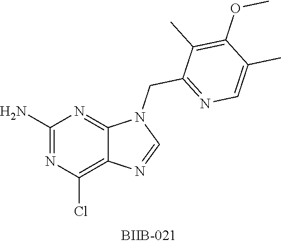 [SMILES: COc1c(C)cnc(Cn2cnc3c(Cl)nc(N)nc32)c1C]

In some embodiments, BIIB is administered orally. In other embodiments, BIIB is administered once a day orally. In other embodiments, BIIB is administered twice a day orally. In other embodiments, BIIB is administered once a week orally. In other embodiments, BIIB is administered thrice a week orally.

BIIB and/or structurally related compounds are described in PCT International Patent Application WO2014/056953, the contents of which are hereby incorporated by reference for all purposes and the specific purposes identified herein. In some embodiments, such compounds are represented by any one or more of the structures shown in Table 15. Any one of the compounds depicted in Table 15 is suitable for use in the methods of the present disclosure.

TABLE 15
Compound

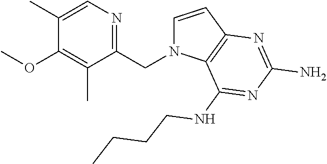 [SMILES: CCCCNc1nc(N)nc2ccn(Cc3ncc(C)c(OC)c3C)c12]

In some embodiments, the compound is Tenofovir, which is chemically designated (((1-(6-amino-9H-purin-9-yl)propan-2-yl)oxy)methyl)phosphonic acid. Tenofovir is represented by the structure:

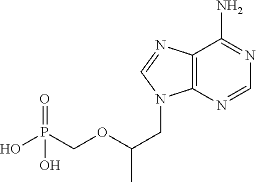 [SMILES: CC(Cn1cnc2c(N)ncnc21)OCP(=O)(O)O]

Tenofovir is marketed as Tenofovir disoproxil, sold under the trade name Viread, among others, for the treatment of chronic hepatitis B and to prevent and treat HIV/AIDS. The structure of tenofovir disoproxil is shown below.

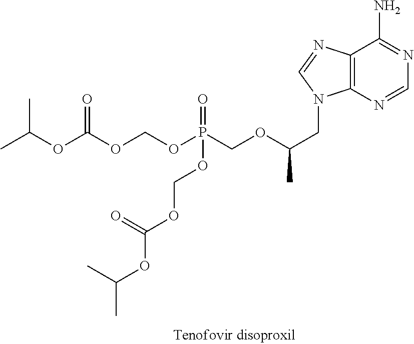 [SMILES: CC(C)OC(=O)OCOP(=O)(CO[C@H](C)Cn1cnc2c(N)ncnc21)OCOC(=O)OC(C)C]

A second generation tenofovir is Tenofovir Exalidex (TXL), which is being studied in clinical trials for renal impairment. The structure of Tenofovir Exalidex is represented below.

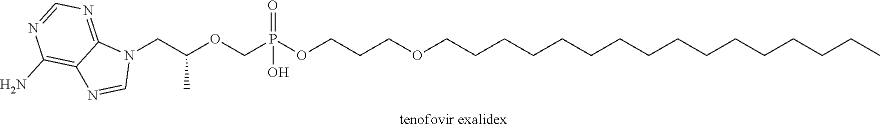 [SMILES: CCCCCCCCCCCCCCCCOCCCOP(=O)(O)CO[C@H](C)Cn1cnc2c(N)ncnc21]

Another tenofovir prodrug is tenofovir alafenamide (marketed as a hemifumarate salt under the trade name VEMLIDY®) the structure of which is shown below:

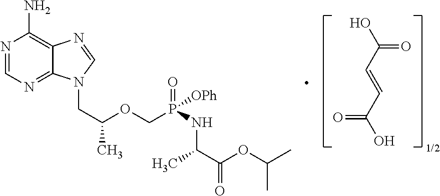

In some embodiments, tenofovir and/or prodrugs thereof is administered orally. In some embodiments, tenofovir and/or prodrugs thereof is administered orally as a tablet.

Tenofovir and/or structurally related compounds are described in PCT International Patent Application WO 2008/005555, the contents of which are hereby incorporated by reference for all purposes and the specific purposes identified herein. In some embodiments, such compounds are represented by any one or more of the structures shown in Table 16. Any one of the compounds depicted in Table 16 is suitable for use in the methods of the present disclosure.

TABLE 16
Compound

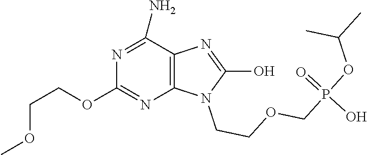 [SMILES: COCCOc1nc(N)c2nc(O)n(CCOCP(=O)(O)OC(C)C)c2n1]

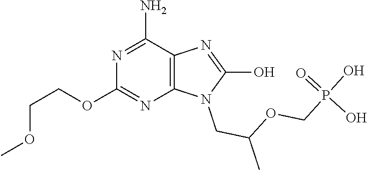 [SMILES: COCCOc1nc(N)c2nc(O)n(CC(C)OCP(=O)(O)O)c2n1]

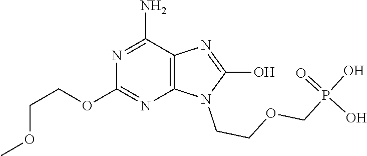 [SMILES: COCCOc1nc(N)c2nc(O)n(CCOCP(=O)(O)O)c2n1]

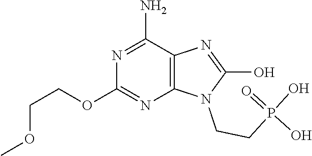 [SMILES: COCCOc1nc(N)c2nc(O)n(CCP(=O)(O)O)c2n1]

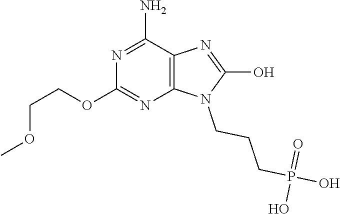 [SMILES: COCCOc1nc(N)c2nc(O)n(CCCP(=O)(O)O)c2n1]

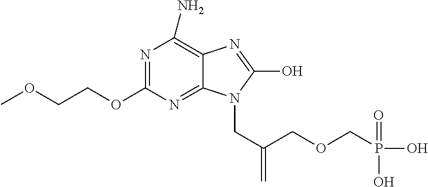 [SMILES: C=C(COCP(=O)(O)O)Cn1c(O)nc2c(N)nc(OCCOC)nc21]

### Methods of Treatment

In certain embodiments, the compositions and methods of the present disclosure are useful for the prevention and/or treatment of symptoms of SARS-CoV-19 infections. In certain embodiments, the compositions and methods of the present disclosure are useful for the prevention and/or treatment of acute inflammatory responses. In certain embodiments, the compositions and methods of the present disclosure are useful for the prevention and/or treatment of acute inflammatory responses, e.g., cytokine storms that are associates with a coronavirus infection.

The Toll Like Receptor (TLR) family plays an important role in pathogen recognition and activation of innate immunity. TLRs recognize pathogen-associated molecular patterns (PAMPs) that are expressed on infectious agents, and mediate the production of cytokines necessary for the development of effective immunity. The activation of TLRs establishes antiviral innate immune responses and coordinates the development of long-lasting adaptive immunity in order to control viral pathogenesis. Thus, TLRs are promising targets as therapeutics for the treatment of viral infections that result in inflammatory damage.

TLR7 recognizes single-stranded RNA in endosomes, which is a common feature of viral genomes which are internalized by macrophages and dendritic cells. TLR7 recognizes single-stranded RNA of viruses such as HIV and HCV. Due to their ability to induce robust production of anti-cancer cytokines such as interleukin-12, TLR7 agonists been investigated for cancer immunotherapy. TLR-7 agonists have further been investigated s anti-viral therapies such as human hepatitis-B (HBV) and human hepatitis-C(HCV), as well as human papilloma virus (HPV). TLR-7 agonists have further been used topically for the treatment of actinic keratosis (AK) and skin cancers.

TLR-8 is an endosomal receptor that recognizes single stranded RNA (ssRNA), and can recognize ssRNA viruses such as Influenza, Sendai, and Coxsackie B viruses. TLR8 binding to the viral RNA recruits MyD88 and leads to activation of the transcription factor NF-κB and an antiviral response. TLR8 recognizes single-stranded RNA of viruses such as HIV and HCV TLR8 agonists have undergone clinical trials as immune stimulants in combination therapy for some cancers.

In some embodiments, TLR-7 and TLR-8 agonists act as immunostimulants and are useful for treatment of immune responses associated with coronavirus infections, in particular SARS-Cov-19.

Thus, in some embodiments, the present disclosure relates to a method of treating or alleviating at least one symptom of a coronavirus infection in a subject, by administering to the subject a therapeutically effective amount of a Toll-Like Receptor (TLR)-7 or TLR-8 agonist. In some embodiments, the subject is a human.

In some embodiments, the symptom is fever. In other embodiments, the symptom is cough. In other embodiments, the symptom is dry cough. In other embodiments, the symptom is tiredness. In other embodiments, the symptom is sore throat. In other embodiments, the symptom is diarrhea. In other embodiments, the symptom is conjunctivitis. In other embodiments, the symptom is headache. In other embodiments, the symptom is loss of taste. In other embodiments, the symptom is loss of smell. In other embodiments, the symptom is a rash. In other embodiments, the symptom is difficulty breathing. In other embodiments, the symptom is shortness of breath. In other embodiments, the symptom is chest pain. In other embodiments, the symptom is chest pressure. In other embodiments, the symptom is Acute Respiratory Distress Syndrome (ARDS). In other embodiments, the symptom is organ failure. In other embodiments, the symptom is multiple organ failure. In other embodiments, the symptom is any combination of the foregoing.

In some embodiments, the present disclosure relates to a method of treating an acute inflammatory condition in a subject infected with a coronavirus, the method comprising the step of administering to the subject a therapeutically effective amount of a Toll-Like Receptor (TLR)-7 or TLR-8 agonist. In some embodiments, the inflammatory condition comprises a cytokine storm. In some embodiments, the subject is a human.

In some embodiments, the present disclosure relates to a method of preventing a cytokine storm in a subject infected with a coronavirus, the method comprising the step of administering to the subject a therapeutically effective amount of a Toll-Like Receptor (TLR)-7 or TLR-8 agonist. In some embodiments, the subject is a human.

In some embodiments, the present disclosure relates to a method of reducing or arresting viral load in a subject infected with a coronavirus, the method comprising the step of administering to the subject a therapeutically effective amount of a Toll-Like Receptor (TLR)-7 or TLR-8 agonist. In some embodiments, the subject is a human.

Viral load can be measured by any viral diagnostic equipment or technique known in the art. A wide variety of samples can be used for virological testing. Such samples include, but are not limited to, upper respiratory swabs (nasopharyngeal swabs, nasopharyngeal wash/aspirate, oropharyngeal swabs, saliva) and lower respiratory specimens (sputum, bronchoalveolar lavage, lung tissue), as well as stool, rectal swabs, blood, skin, urine, semen, faeces, cerebrospinal fluid, tissue (e.g., biopsies), and the like. Techniques for measuring viral load include, but are not limited to, nucleic acid amplification-based tests (NATs) or non-nucleic acid-based tests. Examples of NATs include, but are not limited to, PCR (polymerase chain reaction), reverse transcription polymerase chain reaction (RT-PCR), and nucleic acid sequence-based amplification (NASBA). Viral load is typically reported as copies the virus in a milliliter (mL) of blood. Changes in viral load are usually reported as a log change (in powers of 10). For example, a three-log increase in viral load (3 log 10) is an increase of 103 or 1,000 times the previously reported level, while a drop from 500,000 to 500 copies would be a three-log-drop.

In one embodiment, the subject is infected with a coronavirus. In some embodiments, the coronavirus is selected from the group consisting of 229E (alpha coronavirus), NL63 (alpha coronavirus), OC43 (beta coronavirus), HKU1 (beta coronavirus), MERS-CoV (beta coronavirus that causes Middle East Respiratory Syndrome, or MERS), SARS-CoV (the beta coronavirus that causes severe acute respiratory syndrome, or SARS) SARS-CoV-2 (the novel coronavirus that causes coronavirus disease 2019, or COVID-19, also referred to herein as SARS-Covid-19). In some embodiments, the coronavirus is a severe acute respiratory syndrome coronavirus (SARS-CoV). In some embodiments, the coronavirus is a novel virus 2019-nCoV (SARS-CoV-19). In some embodiments, the coronavirus is a Middle East respiratory syndrome coronavirus (MERS-CoV). In one preferred embodiment, the coronavirus is SARS-CoV-19.

### Pharmaceutical Compositions

The present disclosure thus provides pharmaceutical compositions comprising TLR 7/TLR 8 agonists and a pharmaceutically acceptable carrier. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the TLR-7 or TLR-8 agonists may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants, leaves, flowers, and fruits, and combinations thereof. Exemplary flavouring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The TLR-7 or TLR-8 agonists of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and □-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop, a patch or a cream. For topical administration to body surfaces using, for example, creams, gels, drops, ointments and the like, the compounds of the present disclosure can be prepared and applied in a physiologically acceptable diluent with or without a pharmaceutical carrier.

Adjuvants for topical or gel base forms may include, for example, sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water, benzyl alcohol, methylparaben, and propylparaben. Additional additives may be selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like. Examples of suitable topical dosage forms may be found in e.g., Tarun Garg, Goutam Rath & Amit K. Goyal (2015) Comprehensive review on additives of topical dosage forms for drug delivery, Drug Delivery, 22:8, 969-987, the contents of which are hereby incorporated by reference in their entirety.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject (e.g., delays the onset of and/or reduces the severity of one or more of the symptoms associated with a coronavirus, e.g., SARS-Covid-19.

A TLR-7 or TLR-8 agonist described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5 g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A TLR-7 or TLR-8 agonist described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5 mg to about 5 mg, from about 0.5 mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the TLR-7 or TLR-8 agonist is administered for a period of at least one day. In other embodiments, the TLR-7 or TLR-8 agonist is administered for a period of at least 2 days. In other embodiments, the TLR-7 or TLR-8 agonist is administered for a period of at least 3 days. In other embodiments, the TLR-7 or TLR-8 agonist is administered for a period of at least 4 days. In other embodiments, the TLR-7 or TLR-8 agonist is administered for a period of at least 5 days. In other embodiments, the TLR-7 or TLR-8 agonist is administered for a period of at least 6 days. In other embodiments, the TLR-7 or TLR-8 agonist is administered for a period of at least 7 days. In other embodiments, the TLR-7 or TLR-8 agonist is administered for a period of at least 10 days. In other embodiments, the TLR-7 or TLR-8 agonist is administered for a period of at least 14 days. In other embodiments, the TLR-7 or TLR-8 agonist is administered for a period of at least one month. In some embodiments, the TLR-7 or TLR-8 agonist is administered chronically for as long as the treatment is needed.

In one or more further implementations, the following embodiments are provided: understood

Embodiment 1. A method of treating or alleviating at least one symptom of a coronavirus infection in a subject, the method comprising the step of administering to the subject a therapeutically effective amount of a Toll-Like Receptor (TLR)-7 or TLR-8 agonist.

Embodiment 2. The method according to any of the preceding embodiments, wherein the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure.

Embodiment 3. A method of preventing or treating an acute inflammatory condition in a subject infected with a coronavirus, the method comprising the step of administering to the subject a therapeutically effective amount of a Toll-Like Receptor (TLR)-7 or TLR-8 agonist.

Embodiment 4. The method according to any of the preceding embodiments, wherein the inflammatory condition comprises a cytokine storm.

Embodiment 5. A method of preventing or treating a cytokine storm in a subject infected with a coronavirus, the method comprising the step of administering to the subject a therapeutically effective amount of a Toll-Like Receptor (TLR)-7 or TLR-8 agonist.

Embodiment 6. A method of reducing or arresting viral load in a subject infected with a coronavirus, the method comprising the step of administering to the subject a therapeutically effective amount of a Toll-Like Receptor (TLR)-7 or TLR-8 agonist.

Embodiment 7. The method according to any of the preceding embodiments, wherein the coronavirus is selected from the group consisting of severe acute respiratory syndrome coronavirus (SARS-CoV), novel virus 2019-nCoV (SARS-CoV-19), and the Middle East respiratory syndrome coronavirus (MERS-CoV).

Embodiment 8. The method according to any of the preceding embodiments, wherein the coronavirus is SARS-CoV-19.

Embodiment 9. The method according to any of the proceeding embodiments, wherein the TLR-7 or TLR-8 agonist is selected from the group consisting of:
4-amino-2-butoxy-8-(3-(pyrrolidin-1-ylmethyl)benzyl)-7,8-dihydropteridin-6(5H)-one (vesatolimod);
7-allyl-2-amino-9-((2R,3R,4S,5R)-3,4-dihydroxy-5-(hydroxymethyl) tetrahydrofuran-2-yl)-7,9-dihydro-1H-purine-6,8-dione (loxoribine);
2-amino-N,N-dipropyl-8-(4-(pyrrolidine-1-carbonyl)phenyl)-3H-benzo[b]azepine-4-carboxamide (motolimod);
3-[5-amino-2-[2-[4-[2-(3,3-difluoro-3-phosphonopropoxy)ethoxy]-2-methylphenyl]ethyl]benzo[f][1,7]naphthyridin-8-yl]propanoic acid (LHC-165);
1-(4-amino-2-(ethoxymethyl)-1H-imidazo[4,5-c]quinolin-1-yl)-2-methylpropan-2-01 (resiquimod);
6-amino-2-(pentan-2-yloxy)-9-(5-(piperidin-1-yl)pentyl)-7,9-dihydro-8H-purin-8-one (GSK-2245035);
N-(4-((4-amino-2-butyl-1H-imidazo[4,5-c]quinolin-1-yl)oxy)butyl)stearamide (telratolimod);
N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione (isatoribine);
N-[4-(4-amino-2-ethyl-1H-imidazo[4,5-c]quinolin-1-yl)butyl]-methanesulfonamide (3M-852A);
1-isobutyl-1H-imidazo[4,5-c]quinolin-4-amine (imiquimod);
2-(4-((6-amino-2-(2-methoxyethoxy)-8-oxo-7H-purin-9(8H)methyl)benzamido)-ethyl-2,3-bis(oleoyloxy)propyl phosphate (TMX-202);
methyl 2-(3-(((3-(6-amino-2-butoxy-8-oxo-7,8-dihydro-9H-purin-9-yl)propyl)(3-morpholinopropyl)amino)methyl) phenyl) acetate (AZD-8848);
4-amino-1-benzyl-6-trifluoromethyl-1,3-dihydro-imidazo[4,5-c]pyridine-2-one (PF-4171455);
1-isobutyl-1H-imidazo[4,5-c][1,5]naphthyridin-4-amine (epetirimod);
1-isobutyl-2-methyl-1H-imidazo[4,5-c][1,5]naphthyridin-4-amine (sotirimod);
1-isobutyl-2-methyl-1H-imidazo[4,5-c][1,5]naphthyridin-4-amine (BIIB-021);
(((1-(6-amino-9H-purin-9-yl)propan-2-yl)oxy)methyl)phosphonic acid (tenofovir);
   a tenefovir prodrug selected from the group consisting of tenofovir disoproxil and tenofovir exalidex;
   and salts and any combinations thereof.

Embodiment 10. The method according to any of the preceding embodiments, wherein the TLR-7 or TLR-8 agonist is selected from the group consisting of a compound of any one of Table 1, Table 2, Table 3, Table 4, Table 5, Table 6, Table 7, Table 8, Table 9, Table 10, Table 11, Table 12, Table 13, Table 14, Table 15 or Table 16.

Embodiment 11. The method according to any of the preceding embodiments, wherein the TLR-7 or TLR-8 agonist is administered according to a dosing regimen selected from the group consisting of once daily (q.d.), twice daily (b.i.d.) thrice daily (t.i.d.), once a week, twice a week, three times a week, once every 2 weeks, once every three weeks, or once a month.

Embodiment 12. The method according to any of the preceding embodiments, wherein the TLR-7 or TLR-8 agonist is administered in a pharmaceutical composition, wherein the composition further comprises at least one pharmaceutically acceptable excipient.

Embodiment 13. The method according to any of the preceding embodiments, wherein the TLR-7 or TLR-8 agonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

Embodiment 14. The method according to any of the preceding embodiments, wherein the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

Embodiment 15. The method according to any of the preceding embodiments, wherein the subject is a human.

Embodiment 16. A topical pharmaceutical composition in a form selected from the group consisting of ointment, a gel, a drop, a patch and a cream, the composition comprising a TLR-7 or TLR-8 agonist and at least one topically acceptable excipient, wherein the TLR-7 or TLR-7 agonist is selected from the group consisting of
4-amino-2-butoxy-8-(3-(pyrrolidin-1-ylmethyl)benzyl)-7,8-dihydropteridin-6(5H)-one (vesatolimod);
7-allyl-2-amino-9-((2R,3R,4S,5R)-3,4-dihydroxy-5-(hydroxymethyl) tetrahydrofuran-2-yl)-7,9-dihydro-1H-purine-6,8-dione (loxoribine);
2-amino-N,N-dipropyl-8-(4-(pyrrolidine-1-carbonyl)phenyl)-3H-benzo[b]azepine-4-carboxamide (motolimod);
3-[5-amino-2-[2-[4-[2-(3,3-difluoro-3-phosphonopropoxy)ethoxy]-2-methylphenyl]ethyl]benzo[f][1,7]naphthyridin-8-yl]propanoic acid (LHC-165);
1-(4-amino-2-(ethoxymethyl)-1H-imidazo[4,5-c]quinolin-1-yl)-2-methylpropan-2-01 (resiquimod);
6-amino-2-(pentan-2-yloxy)-9-(5-(piperidin-1-yl)pentyl)-7,9-dihydro-8H-purin-8-one (GSK-2245035);
N-(4-((4-amino-2-butyl-1H-imidazo[4,5-c]quinolin-1-yl)oxy)butyl)stearamide (telratolimod);
N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione (isatoribine);
N-[4-(4-amino-2-ethyl-1H-imidazo[4,5-c]quinolin-1-yl)butyl]-methanesulfonamide (3M-852A);
1-isobutyl-1H-imidazo[4,5-c]quinolin-4-amine (imiquimod);
2-(4-((6-amino-2-(2-methoxyethoxy)-8-oxo-7H-purin-9(8H)methyl)benzamido)-ethyl-2,3-bis(oleoyloxy)propyl phosphate (TMX-202);
methyl 2-(3-(((3-(6-amino-2-butoxy-8-oxo-7,8-dihydro-9H-purin-9-yl)propyl)(3-morpholinopropyl)amino)methyl) phenyl) acetate (AZD-8848);
4-amino-1-benzyl-6-trifluoromethyl-1,3-dihydro-imidazo[4,5-c]pyridine-2-one (PF-4171455);
1-isobutyl-1H-imidazo[4,5-c][1,5]naphthyridin-4-amine (epetirimod);
1-isobutyl-2-methyl-1H-imidazo[4,5-c][1,5]naphthyridin-4-amine (sotirimod);
1-isobutyl-2-methyl-1H-imidazo[4,5-c][1,5]naphthyridin-4-amine (BIIB-021);
(((1-(6-amino-9H-purin-9-yl)propan-2-yl)oxy)methyl)phosphonic acid (tenofovir);
   a tenefovir prodrug selected from the group consisting of tenofovir disoproxil and tenofovir exalidex;
   and salts and any combinations thereof.

Embodiment 17. The topical pharmaceutical composition of any of the preceding embodiments, in a form selected from the group consisting of ointment, a gel, a drop, a patch and a cream, the pharmaceutical composition comprising a TLR-7 or TLR-8 agonist and at least one topically acceptable excipient, wherein the TLR-7 or TLR-7 agonist is selected from the group consisting of a compound of any one of Table 1, Table 2, Table 3, Table 4, Table 5, Table 6, Table 7, Table 8, Table 9, Table 10, Table 11, Table 12, Table 13, Table 14, Table 15 or Table 16.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

The foregoing description of the specific embodiments will so fully reveal the general nature of the disclosure that others can, by applying knowledge within the skill of the relevant art(s) (including the contents of the documents cited and incorporated by reference herein for all purposes), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

What is claimed:

1. A method of treating or alleviating at least one symptom of a coronavirus infection in a subject, the method comprising the step of administering to the subject a therapeutically effective amount of a Toll-Like Receptor (TLR)-7 or TLR-8 agonist.

2. The method according to claim 1, wherein the symptom is selected from the group consisting of fever, cough, tiredness, sore throat, diarrhea, conjunctivitis, headache, loss of taste, loss of smell, rash, difficulty breathing, shortness of breath, chest pain, chest pressure, Acute Respiratory Distress Syndrome (ARDS) and organ failure.

3. A method of preventing or treating an acute inflammatory condition in a subject infected with a coronavirus, the method comprising the step of administering to the subject a therapeutically effective amount of a Toll-Like Receptor (TLR)-7 or TLR-8 agonist.

4. The method according to claim 3, wherein the inflammatory condition comprises a cytokine storm.

5. The method according to claim 3, wherein the acute inflammatory condition is a cytokine storm in a subject infected with a coronavirus.

6. The method according to claim 3, wherein the therapeutically effective amount of a Toll-Like Receptor (TLR)-7 or TLR-8 agonist is sufficient in reducing or arresting viral load in a subject infected with a coronavirus.

7. The method according to claim 4, wherein the coronavirus is selected from the group consisting of severe acute respiratory syndrome coronavirus (SARS-CoV), novel virus 2019-nCoV (SARS-CoV-19), and the Middle East respiratory syndrome coronavirus (MERS-CoV).

8. The method according to claim 7, wherein the coronavirus is SARS-CoV-19.

9. The method according to claim 4, wherein the TLR-7 or TLR-8 agonist is selected from the group consisting of:
4-amino-2-butoxy-8-(3-(pyrrolidin-1-ylmethyl)benzyl)-7,8-dihydropteridin-6(5H)-one (vesatolimod);
7-allyl-2-amino-9-((2R,3R,4S,5R)-3,4-dihydroxy-5-(hydroxymethyl) tetrahydrofuran-2-yl)-7,9-dihydro-1H-purine-6,8-dione (loxoribine);
2-amino-N,N-dipropyl-8-(4-(pyrrolidine-1-carbonyl)phenyl)-3H-benzo[b]azepine-4-carboxamide (motolimod);
3-[5-amino-2-[2-[4-[2-(3,3-difluoro-3-phosphonopropoxy)ethoxy]-2-methylphenyl]ethyl]benzo[f][1,7]naphthyridin-8-yl]propanoic acid (LHC-165);
1-(4-amino-2-(ethoxymethyl)-1H-imidazo[4,5-c]quinolin-1-yl)-2-methylpropan-2-ol (resiquimod);
6-amino-2-(pentan-2-yloxy)-9-(5-(piperidin-1-yl)pentyl)-7,9-dihydro-8H-purin-8-one (GSK-2245035);
N-(4-((4-amino-2-butyl-1H-imidazo[4,5-c]quinolin-1-yl)oxy)butyl)stearamide (telratolimod);
N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione (isatoribine);
N-[4-(4-amino-2-ethyl-1H-imidazo[4,5-c]quinolin-1-yl)butyl]-methanesulfonamide (3M-852A);
1-isobutyl-1H-imidazo[4,5-c]quinolin-4-amine (imiquimod);
2-(4-((6-amino-2-(2-methoxyethoxy)-8-oxo-7H-purin-9(8H)methyl)benzamido)-ethyl-2,3-bis(oleoyloxy)propyl phosphate (TMX-202);
methyl 2-(3-(((3-(6-amino-2-butoxy-8-oxo-7,8-dihydro-9H-purin-9-yl)propyl)(3-morpholinopropyl)amino)methyl) phenyl) acetate (AZD-8848);
4-amino-1-benzyl-6-trifluoromethyl-1,3-dihydro-imidazo[4,5-c]pyridine-2-one (PF-4171455);
1-isobutyl-1H-imidazo[4,5-c][1,5]naphthyridin-4-amine (epetirimod);
1-isobutyl-2-methyl-1H-imidazo[4,5-c][1,5]naphthyridin-4-amine (sotirimod);
1-isobutyl-2-methyl-1H-imidazo[4,5-c][1,5]naphthyridin-4-amine (BIIB-021);
(((1-(6-amino-9H-purin-9-yl)propan-2-yl)oxy)methyl)phosphonic acid (tenofovir);
a tenefovir prodrug selected from the group consisting of tenofovir disoproxil and tenofovir exalidex;
and salts and any combinations thereof.

10. The method according to claim 1, wherein the TLR-7 or TLR-8 agonist is selected from the group consisting of a compound of any one of Table 1, Table 2, Table 3, Table 4, Table 5, Table 6, Table 7, Table 8, Table 9, Table 10, Table 11, Table 12, Table 13, Table 14, Table 15 or Table 16.

11. The method according to claim 9, wherein the TLR-7 or TLR-8 agonist is administered according to a dosing regimen selected from the group consisting of once daily (q.d.), twice daily (b.i.d.) thrice daily (t.i.d.), once a week, twice a week, three times a week, once every 2 weeks, once every three weeks, or once a month.

12. The method according to claim 11, wherein the TLR-7 or TLR-8 agonist is administered in a pharmaceutical composition, wherein the composition further comprises at least one pharmaceutically acceptable excipient.

13. The method according to claim 12, wherein the TLR-7 or TLR-8 agonist is administered in a form selected from the group consisting of a solution, a suspension, a syrup, an emulsion, a dispersion, a tablet, a pill, a capsule, a pellet, granules, a powder, an ointment, an elixir, a wafer, coated or uncoated beads, a lozenge, a sachet, a cachet, a depot system, a patch, an aerosol, an oil, an ointment, a suppository, a gel, and a cream.

14. The method according to claim 13, wherein the pharmaceutical composition is formulated for oral, topical, mucosal, intranasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural, sublingual oral, intranasal, intravenous, intraarterial, intrathecal, vaginal, rectal or subcutaneous administration.

15. The method according to claim 4, wherein the subject is a human.

16. A topical pharmaceutical composition in a form selected from the group consisting of ointment, a gel, a drop, a patch and a cream, the composition comprising a TLR-7 or TLR-8 agonist and at least one topically acceptable excipient, wherein the TLR-7 or TLR-7 agonist is selected from the group consisting of
4-amino-2-butoxy-8-(3-(pyrrolidin-1-ylmethyl)benzyl)-7,8-dihydropteridin-6(5H)-one (vesatolimod);
7-allyl-2-amino-9-((2R,3R,4S,5R)-3,4-dihydroxy-5-(hydroxymethyl) tetrahydrofuran-2-yl)-7,9-dihydro-1H-purine-6,8-dione (loxoribine);
2-amino-N,N-dipropyl-8-(4-(pyrrolidine-1-carbonyl)phenyl)-3H-benzo[b]azepine-4-carboxamide (motolimod);
3-[5-amino-2-[2-[4-[2-(3,3-difluoro-3-phosphonopropoxy)ethoxy]-2-methylphenyl]ethyl]benzo[f][1,7]naphthyridin-8-yl]propanoic acid (LHC-165);
1-(4-amino-2-(ethoxymethyl)-1H-imidazo[4,5-c]quinolin-1-yl)-2-methylpropan-2-ol (resiquimod);
6-amino-2-(pentan-2-yloxy)-9-(5-(piperidin-1-yl)pentyl)-7,9-dihydro-8H-purin-8-one (GSK-2245035);
N-(4-((4-amino-2-butyl-1H-imidazo[4,5-c]quinolin-1-yl)oxy)butyl)stearamide (telratolimod);
N-5-amino-3-[(2R,3R,4S,5R)-3,4-dihydroxy-5-(hydroxymethyl)oxolan-2-yl]-6H-[1,3] thiazolo[4,5-d]pyrimidine-2,7-dione (isatoribine);
N-[4-(4-amino-2-ethyl-1H-imidazo[4,5-c]quinolin-1-yl)butyl]-methanesulfonamide (3M-852A);
1-isobutyl-1H-imidazo[4,5-c]quinolin-4-amine (imiquimod);
2-(4-((6-amino-2-(2-methoxyethoxy)-8-oxo-7H-purin-9(8H)methyl)benzamido)-ethyl-2,3-bis(oleoyloxy)propyl phosphate (TMX-202);
methyl 2-(3-(((3-(6-amino-2-butoxy-8-oxo-7,8-dihydro-9H-purin-9-yl)propyl)(3-morpholinopropyl)amino)methyl) phenyl) acetate (AZD-8848);
4-amino-1-benzyl-6-trifluoromethyl-1,3-dihydro-imidazo[4,5-c]pyridine-2-one (PF-4171455);
1-isobutyl-1H-imidazo[4,5-c][1,5]naphthyridin-4-amine (epetirimod);
1-isobutyl-2-methyl-1H-imidazo[4,5-c][1,5]naphthyridin-4-amine (sotirimod);
1-isobutyl-2-methyl-1H-imidazo[4,5-c][1,5]naphthyridin-4-amine (BIIB-021);
(((1-(6-amino-9H-purin-9-yl)propan-2-yl)oxy)methyl)phosphonic acid (tenofovir);
a tenefovir prodrug selected from the group consisting of tenofovir disoproxil and tenofovir exalidex;
and salts and any combinations thereof.

17. The topical pharmaceutical composition of claim 16, in a form selected from the group consisting of ointment, a gel, a drop, a patch and a cream, the pharmaceutical composition comprising a TLR-7 or TLR-8 agonist and at least one topically acceptable excipient, wherein the TLR-7 or TLR-7 agonist is selected from the group consisting of a compound of any one of Table 1, Table 2, Table 3, Table 4, Table 5, Table 6, Table 7, Table 8, Table 9, Table 10, Table 11, Table 12, Table 13, Table 14, Table 15 or Table 16.
